(19) United States
(12) Patent Application Publication
(10) Pub. No.: US 2013/0071415 A1
(43) Pub. Date: Mar. 21, 2013

(54) Heterocyclic Compounds as Janus Kinase Inhibitors

(71) Applicants: Yarlagadda S. Babu, Birmingham, AL (US); Pravin L. Kotian, Birmingham, AL (US); Minwan Wu, Vestavia Hills, AL (US)

(72) Inventors: Yarlagadda S. Babu, Birmingham, AL (US); Pravin L. Kotian, Birmingham, AL (US); Minwan Wu, Vestavia Hills, AL (US)

(73) Assignee: BioCryst Pharmaceuticals, Inc., Durham, NC (US)

(21) Appl. No.: 13/699,818

(22) PCT Filed: May 27, 2011
(86) PCT No.: PCT/US11/38387

§ 371 Date: Nov. 26, 2012

Related U.S. Application Data
(60) Provisional application No. 61/349,364, filed on May 28, 2010.

(51) Int. Cl. C07D 487/04

(52) U.S. Cl. CPC C07D 487/04
USPC 424/184.1; 544/183; 514/243; 544/235; 514/248

(57) ABSTRACT

The invention provides compounds of formula I:

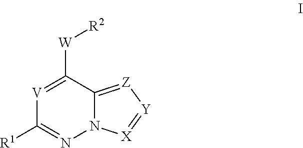

or a salt thereof as described herein. The invention also provides pharmaceutical compositions comprising a compound of formula I, processes for preparing compounds of formula I, intermediates useful for preparing compounds of formula I and therapeutic methods for suppressing an immune response or treating cancer or a hematologic malignancy using compounds of formula I.

### CROSS-REFERENCE TO RELATED APPLICATIONS

This patent application claims the benefit of priority of U.S. application Ser. No. 61/349,364, filed May 28, 2010.

### BACKGROUND OF THE INVENTION

Janus kinase 3 (JAK3) is a cytoplasmic protein tyrosine kinase associated with the common gamma chain (γc), which is an integral component of various cytokine receptors (Elizabeth Kudlacz et al., American Journal of Transplantation, 2004, 4, 51-57).

While effective in the prevention of transplant rejection, commonly used immunosuppressants, such as calcineurin inhibitors, possess a number of significant dose-limiting toxicities, thereby prompting a search for agents with novel mechanisms of action. The inhibition of JAK3 represents an attractive strategy for immunosuppression based upon its limited tissue distribution, lack of constitutive activation and the evidence for its role in immune cell function. JAK3 is a viable target for immunosuppression and transplant rejection. JAK3 specific inhibitors may also be useful for treatment of hematologic and other malignancies that involve pathologic JAK activation.

Currently, there is a need for compounds, compositions and methods that are useful for treating diseases and conditions associated with pathologic JAK activation.

### SUMMARY OF THE INVENTION

In one embodiment, the invention provides a compound of the invention which is a compound of formula I:

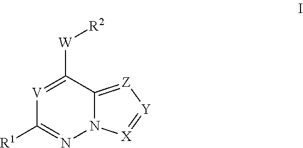

wherein:

W is heteroaryl, heterocycle or aryl, wherein any aryl or heteroaryl of W may be optionally substituted with one or more (e.g. 1, 2, 3, 4 or 5) R_{w} groups and wherein any heterocycle of W may be optionally substituted with one or more groups selected from R_{w} and oxo;

X is N or CRₐ; Y is N or CR_{b}; Z is N or CR_{c}; and V is N or CR_{d} provided that no more than two of X, Y, Z or V is N;

R¹ is H, halogen, —(C₁-C₈)alkyl, —(C₂-C₈)alkenyl, —(C₂-C₈)alkynyl, (C₃-C₈)cycloalkyl, aryl, heteroaryl, heterocycle, NO₂, CN, —OH, —ORₑ, —NR_{f}R_{g}, N₃, SH, —SRₑ, —C(O)Rₕ, —C(O)ORₕ, —C(O)NR_{f}R_{g}, —C(═NRₕ)NR_{f}R_{g}, —NRₕCORₑ, —NRₕC(O)ORₑ, —NRₕC(O)OH, —NRₕS(O)₂Rₑ, —NRₕCONR_{f}R_{g}, —OC(O)NR_{f}R_{g}, —S(O)Rₑ, —S(O)NR_{f}R_{g}, —S(O)₂Rₑ, —S(O)₂OH, or —S(O)₂NR_{f}R_{g}, wherein any aryl or heteroaryl of R¹ may be optionally substituted with one or more (e.g. 1, 2, 3, 4 or 5) R, groups and wherein any —(C₁-C₈)alkyl, (C₃-C₈)cycloalkyl, —(C₂-C₈)alkenyl, —(C₂-C₈)alkynyl or heterocycle of R¹ may be optionally substituted with one or more groups selected from Rᵢ, oxo and=NORₕ;

R² is selected from halogen, aryl, heteroaryl, heterocycle, —(C₁-C₈)alkyl, —(C₂-C₈)alkenyl, —(C₂-C₈)alkynyl, (C₃-C₈)cycloalkyl, OH, CN, —OR_{z}, —Oaryl, —Oheterocycle, —Oheteroaryl, —OC(O)R_{z}, —OC(O)NR_{z1}R_{z2}, SH, —SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, —CHO, —C(O)R_{z}, —C(O)OH, —C(O)OR_{z}, —C(O)NR_{z1}R_{z2}, —C(O)heteroaryl and —C(O)C(O)R_{z}, wherein any —(C₁-C₈)alkyl, —(C₂-C₈)alkenyl, —(C₂-C₈)alkynyl, aryl, —Oaryl, —Oheteroaryl, —Saryl, —Sheteroaryl, —S(O)aryl, —S(O)heteroaryl, —S(O)₂aryl, —S(O)₂heteroaryl, —NHCOaryl, —NHCOheteroaryl, —NHS(O)₂aryl, —C(O)heteroaryl or heteroaryl of R² may be optionally substituted with one or more (e.g. 1, 2, 3, 4 or 5) R_{y} groups and wherein any heterocycle, —Oheterocycle or (C₃-C₈)cycloalkyl of R² may be optionally substituted with one or more (e.g. 1, 2, 3, 4 or 5) groups selected from oxo, ═CHCN and R_{y}; or R² is absent;

Rₐ is H, OH, NO₂, CO₂H, CO₂Rₙ₁, —C(O)NRₙRₒ, —C(O)NHNRₙRₒ, —C(O)NHNHCO₂Rₙ₁, —NHS(O)₂Rₙ₁, —NHCO₂Rₙ₁, —NHCORₙ₂, —NRₙRₒ, halogen or —(C₁-C₆)alkyl wherein —(C₁-C₆)alkyl is optionally substituted with one or more (e.g. 1, 2, 3, 4 or 5) Rₚ groups;

R_{b} is H, OH, NO₂, CO₂H, CO₂Rₙ₁, —C(O)NRₙRₒ, —C(O)NHNRₙRₒ, —C(O)NHNHCO₂Rₙ₁, —NHS (O)₂Rₙ₁, —NHCO₂Rₙ₁, —NHCORₙ₂, halogen or —(C₁-C₆)alkyl wherein —(C₁-C₆)alkyl is optionally substituted with one or more (e.g. 1, 2, 3, 4 or 5) Rₚ groups;

R_{c} is H, OH, NO₂, CO₂H, CO₂Rₙ₁, —C(O)NRₙRₒ, —C(O)NHNRₙRₒ, —C(O)NHNHCO₂Rₙ₁, —NHS(O)₂Rₙ₁, —NHCO₂Rₙ₁, —NHCORₙ₂, —NRₙRₒ, halogen or —(C₁-C₆)alkyl wherein —(C₁-C₆)alkyl is optionally substituted with one or more (e.g. 1, 2, 3, 4 or 5) Rₚ groups;

R_{d} is H, halogen, —(C₁-C₆)alkyl, —(C₂-C₆)alkenyl, —(C₂-C₆)alkynyl, (C₃-C₆)cycloalkyl, aryl, heteroaryl, heterocycle, NO₂, CN, OH, —OR_{q}, —NR_{f}Rₛ, N₃, —SH, —C(O)(C₁-C₆)alkyl, —C(O)(C₂-C₆)alkenyl, —C(O)(C₂-C₆)alkynyl, —C(O)(C₃-C₆)cycloalkyl, —C(O)aryl, —C(O)heteroaryl, —C(O)heterocycle, —C(O)ORₜ, —C(O)NRᵣRₛ, —C(═NRₜ)NRᵣRₛ, —NRₜCOR_{q}, —NRₜC(O)OR_{q}, —NRₜS(O)₂R_{q}, —NRₜCONRᵣRₛ, —OC(O)NRᵣRₛ, —S(O)R_{q}, —S(O)NRᵣRₛ, —S(O)₂R_{q}, —S(O)₂OH, —S(O)₂NRᵣRₛ or —C(═O)C(═O)NH(C₁-C₆)alkyl, wherein any aryl, —C(O)aryl, —C(O)heteroaryl, or heteroaryl of R_{d} may be optionally substituted with one or more (e.g. 1, 2, 3, 4 or 5) Rᵢ groups and wherein any —(C₁-C₆)alkyl, (C₃-C₆)cycloalkyl, —(C₂-C₆)alkenyl, —(C₂-C₆)alkynyl, —C(O)(C₁-C₆)alkyl, —C(O)(C₂-C₆)alkenyl, —C(O)(C₂-C₆)alkynyl, —C(O)(C₃-C₆)cycloalkyl, —C(O)heterocycle or heterocycle of R_{d} may be optionally substituted with one or more (e.g. 1, 2, 3, 4 or 5) groups selected from Rᵢ, oxo and ═NORₜ;

Rₑ is —(C₁-C₆)alkyl, —(C₂-C₆)alkenyl, —(C₂-C₆)alkynyl, (C₃-C₆)cycloalkyl, heterocycle, heteroaryl or aryl;

R_{f} and R_{g} are each independently selected from H, —(C₁-C₆)alkyl, —(C₂-C₆)alkenyl, —(C₂-C₆)alkynyl, (C₃-C₆)cycloalkyl, heterocycle and heteroaryl, wherein any —(C₁-C₆)alkyl of R_{f} or R_{g} may be optionally substituted with one or more (e.g. 1, 2 or 3) groups selected from —C(O)OH and OH; or R_{f} and R_{g} together with the nitrogen to which they are attached form a pyrrolidino, piperidino, piperazino, azetidino, morpholino, or thiomorpholino ring;

Rₕ is H, —(C₁-C₆)alkyl, —(C₂-C₆)alkenyl, —(C₂-C₆)alkynyl, (C₃-C₆)cycloalkyl, heterocycle, heteroaryl or aryl;

each Rᵢ is independently selected from halogen, aryl, heteroaryl, heterocycle, R_{z}, OH, CN, —OR_{z}, —Oaryl, —OC(O)R_{z}, —OC(O)NR_{z1}R_{z2}, SH, —SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, —CHO, —C(O)R_{z}, —C(O)OH, —C(O)OR_{z}, —C(O)NR_{z1}R_{z2} and —C(O)C(O)R_{z}, wherein any aryl, —Oaryl, —Saryl, —S(O)aryl, —S(O)₂aryl, —NHCOaryl or —NHS(O)₂aryl of Rᵢ may be optionally substituted with one or more (e.g. 1, 2, 3, 4 or 5) Rₘ groups;

Rⱼ and Rₖ are each independently selected from H, —(C₁-C₆)alkyl, —(C₂-C₆)alkenyl, —(C₂-C₆)alkynyl, (C₃-C₆)cycloalkyl, aryl, aryl(C₁-C₆)alkyl-, heterocycle and heteroaryl; or Rⱼ and Rₖ together with the nitrogen to which they are attached form a pyrrolidino, piperidino, piperazino, azetidino, morpholino, or thiomorpholino ring;

each Rₘ is independently halogen, aryl, R_{z}, OH, CN, OR_{z}, —Oaryl, —Oheteroaryl, —OC(O)R_{z}, —OC(O)NR_{z1}R_{z2}, SH, SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, CHO, —C(O)R_{z}, —C(O)OH, —C(O)OR_{z}, —C(O)NR_{z1}R_{z2}, —C(O)C(O)R_{z}, heterocycle or heteroaryl;

Rₙ and Rₒ are each independently selected from H, —(C₁-C₆)alkyl, —(C₂-C₆)alkenyl, —(C₂-C₆)alkynyl, (C₃-C₆)cycloalkyl, aryl, aryl(C₁-C₆)alkyl-, heterocycle and heteroaryl, wherein any —(C₁-C₆)alkyl of Rₙ or Rₒ may be optionally substituted with one or more (e.g. 1, 2 or 3) groups selected from —C(O)OH and OH; or Rₙ and Rₒ together with the nitrogen to which they are attached form a pyrrolidino, piperidino, piperazino, azetidino, morpholino, or thiomorpholino ring;

each Rₙ₁ is independently selected from —(C₁-C₆)alkyl, —(C₂-C₆)alkenyl, —(C₂-C₆)alkynyl, (C₃-C₆)cycloalkyl, aryl, aryl(C₁-C₆)alkyl-, heterocycle and heteroaryl;

each Rₙ₂ is independently selected from —(C₁-C₆)alkyl, —(C₂-C₆)alkenyl, —(C₂-C₆)alkynyl, (C₃-C₆)cycloalkyl, aryl, aryl(C₁-C₆)alkyl-, heterocycle and heteroaryl, wherein any —(C₁-C₆)alkyl, —(C₂-C₆)alkenyl, —(C₂-C₆)alkynyl, (C₃-C₆)cycloalkyl, aryl, aryl(C₁-C₆)alkyl-, heterocycle or heteroaryl of Rₙ₂ may be optionally substituted with one or more (e.g. 1, 2, 3, 4 or 5) halogens;

each Rₚ is independently selected from halogen, aryl, heteroaryl, heterocycle, R_{z}, OH, CN, —OR_{z}, —Oaryl, —OC(O)R_{z}, —OC(O)NR_{z1}R_{z1}, oxo, SH, SR₁, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, ═NOR_{z}, —CHO, —C(O)R_{z}, —C(O)OH, —C(O)OR_{z}, —C(O)NR_{z1}R_{z2} and —C(O)C(O)R_{z}, wherein any aryl, —Oaryl, —Saryl, —S(O)aryl, —S(O)₂aryl, —NHCOaryl or —NHS(O)₂aryl, of Rₚ may be optionally substituted with one or more (e.g. 1, 2, 3, 4 or 5) R_{y} groups;

R₁ is —(C₁-C₆)alkyl, —(C₂-C₆)alkenyl, —(C₂-C₆)alkynyl, (C₃-C₆)cycloalkyl, aryl, aryl(C₁-C₆)alkyl-, heterocycle and heteroaryl;

R_{f} and Rₛ are each independently selected from H, —(C₁-C₆)alkyl, —(C₂-C₆)alkenyl, —(C₂-C₆)alkynyl, (C₃-C₆)cycloalkyl, aryl, aryl(C₁-C₆)alkyl-, heterocycle and heteroaryl; or R_{f} and Rₛ together with the nitrogen to which they are attached form a pyrrolidino, piperidino, piperazino, azetidino, morpholino, or thiomorpholino ring;

Rₜ is H, —(C₁-C₆)alkyl, —(C₂-C₆)alkenyl, —(C₂-C₆)alkynyl, (C₃-C₆)cycloalkyl, aryl, aryl(C₁-C₆)alkyl-, heterocycle and heteroaryl;

each R_{w} is independently (C₁-C₆)alkyl, —O(C₁-C₆)alkyl, —C(O)NRⱼRₖ, halogen, CF₃, CN or NHC(O)Rₕ;

each R_{y} is independently halogen, R_{z}, OH, CN, OR_{z}, —Oaryl, —Oheteroaryl, —OC(O)R_{z}, —OC(O)NR_{z1}R_{z2}, SH, SR₁, —Saryl, —Sheteroaryl, —S(O)R_{z}, —S(O)aryl, —S(O)heteroaryl, —S(O)₂OH, —S(O)₂OR_{z}, —S(O)₂R_{z}, —OS(O)₂R_{z}, —S(O)₂Oaryl, —S(O)₂aryl, —OS(O)₂aryl, —S(O)₂heteroaryl, —OS(O)₂heteroaryl, —S(O)₂NR_{z1}R_{z2}, —S(O) NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, ═NOH, ═NOR_{z}, —C(NH₂)(═NCN), CHO, —C(O)R_{z}, —C(O)OH, —C(O)Oaryl, —C(O)OR_{z}, —C(O)NR_{z1}R_{z2}, —C(O)aryl, —OC(O)aryl, —C(O)heteroaryl, —OC(O)heteroaryl, —C(O)C(O)R_{z}, ═CR_{z7}R_{z8}, aryl, heterocycle or heteroaryl, wherein any aryl, —Oaryl, —Oheteroaryl, —Saryl, —Sheteroaryl, —S(O)aryl, —S(O)heteroaryl, —S(O)₂Oaryl, —S(O)₂aryl, —OS(O)₂aryl, —S(O)₂heteroaryl, —OS(O)₂heteroaryl, —NHCOaryl, —NHCOheteroaryl, NHS(O)₂aryl, —C(O)Oaryl, C(O)aryl, —OC(O)aryl, —C(O)heteroaryl, —OC(O)heteroaryl or heteroaryl of R_{y} is optionally substituted with one or more (e.g. 1, 2, 3, 4 or 5) halogen, R_{z}, (C₂-C₆)alkynyl, —OR_{z}, CN, NR_{z1}R_{z2}, —NO₂, —CHO, —Oaryl, —C(O)OR_{z}, —C(O)OH, —NHCOR_{z}, —NHS(O)₂R_{z}, —NHS(O)₂aryl, —NHS(O)₂heteroaryl, —C(O)NR_{z1}R_{z2}, —NHCONR_{z1}R_{z2}, —NHC(O)OR_{z}, —NHCOaryl, —NHCOheteroaryl, —NHC(O)OR_{z}, —(C₂-C₆)alkynyl, —S(O)₂NR_{z1}R_{z2}, —S(O)₂R_{z}, —S(O)₂aryl, —S(O)₂heteroaryl, —S(O)₂(C₃-C₆)cycloalkyl, —S(O)R_{z}, —S(O)aryl, —S(O)heteroaryl, —S(O)(C₃-C₆)cycloalkyl, —SR_{z}, —S(C₁-C₆)alkyl, aryl, heteroaryl or heterocycle, wherein aryl, —Oaryl, —NHS(O)₂aryl, —NHS(O)₂heteroaryl, —NHCOaryl, —NHCOheteroaryl, —S(O)₂aryl, —S(O)₂heteroaryl, —S(O)aryl, —S(O)heteroaryl or heteroaryl is optionally substituted with one or more (e.g. 1, 2, 3, 4 or 5) halogen, CF₃, CN or (C₁-C₃)alkyl and, wherein any heterocycle of R_{y} is optionally substituted with one or more (e.g. 1, 2, 3, 4 or 5) oxo, R_{z}, —S(O)₂R_{z}, —S(O)₂aryl, —S(O)₂heteroaryl, —C(O)R_{z}, —C(O)aryl, —C(O)heteroaryl or heteroaryl wherein —S(O)₂aryl, —S(O)₂heteroaryl, —C(O)aryl, —C(O)heteroaryl or heteroaryl is optionally substituted with one or more(e.g. 1, 2, 3, 4 or 5) halogen or (C₁-C₃)alkyl;

each R_{z} is independently —(C₁-C₆)alkyl or (C₃-C₆)cycloalkyl wherein —(C₁-C₆)alkyl may be optionally substituted with one or more (e.g. 1, 2, 3, 4 or 5) R_{z4} groups and, wherein (C₃-C₆)cycloalkyl may be optionally substituted with one or more (e.g. 1, 2, 3, 4 or 5) groups selected from R_{z4}, —(C₁-C₆)alkyl, —(C₁-C₆)alkylCN and —(C₁-C₆)alkylOH;

R_{z1} and R_{z2} are each independently selected from H, —(C₁-C₆)alkyl, —(C₂-C₆)alkenyl, —(C₂-C₆)alkynyl, (C₃-C₆)cycloalkyl, aryl, heterocycle and heteroaryl, wherein any —(C₁-C₆)alkyl, —(C₂-C₆)alkenyl or —(C₂-C₆)alkynyl of R_{z1} or R_{z2} may be optionally substituted with one or more (e.g. 1, 2, 3, 4 or 5) R_{z3} groups and wherein aryl or heteroaryl of R_{z1} or R_{z2} may be optionally substituted with one or more (e.g. 1, 2, 3, 4 or 5) —(C₁-C₆)alkyl or R_{z3} groups, and wherein any heterocycle or (C₃-C₆)cycloalkyl of R_{z1} or R_{z2} may be optionally substituted with or more (e.g. 1, 2, 3, 4 or 5) —(C₁-C₆)alkyl, oxo or R_{z3} groups; or R_{z1} and R_{z2} together with the nitrogen to which they are attached form a cyclic amino optionally substituted with one or more (e.g. 1, 2, 3, 4 or 5) —(C₁-C₆)alkyl, oxo or R_{z3} groups;

each R_{z3} is independently selected from halogen, CN, CF₃, NR_{z5}R_{z6}, OH, —O(C₁-C₆)alkyl, —C(O)NR_{z5}R_{z6}, —C(O)(C₁-C₆)alkyl, aryl, heterocycle and heteroaryl, wherein any heterocycle of R_{z3} may be substituted with one or more (e.g. 1, 2, 3, 4 or 5) —(C₁-C₆)alkyl;

each R_{A} is independently selected from halogen, CN, OH, —NR_{z5}R_{z6}, —SCN, —O(C ₁-C₆)alkyl, —Sheteroaryl, —S(O)aryl, —S(O)₂aryl, —Oaryl, —C(O)NR_{z5}R_{z6}, (C₃-C₆)cycloalkyl, —CH₂NHCOaryl, —CH₂OCH₂aryl, biphenyl, aryl, heterocycle and heteroaryl, wherein any aryl, heteroaryl, Sheteroaryl, —S(O)aryl, —S(O)₂aryl, —Oaryl, —CH₂NHCOaryl, —CH₂OCH₂aryl, biphenyl or heterocycle of R_{A} may be optionally substituted with one or more (e.g. 1, 2, 3, 4 or 5) halogen, CN, —(C₁-C₆)alkyl, —NH₂, —NHheteroaryl, —NHS(O)₂(C₁-C₆)alkyl or —O(C₁-C₆)alkyl;

R_{z5} and R_{z6} are each independently selected from H or —(C₁-C₆)alkyl wherein alkyl is optionally substituted with NH₂; and

R_{z7} and R_{z8} together with the atom to which they are attached form a (C₃-C₆)cycloalkyl;

or a salt thereof.

The invention also provides a pharmaceutical composition comprising a compound of formula I or a pharmaceutically acceptable salt thereof, in combination with a pharmaceutically acceptable diluent or carrier.

The invention also provides method for treating a disease or condition associated with pathologic JAK activation (e.g. a cancer, a hematologic malignancy or other malignancy) in a mammal (e.g. a human), comprising administering a compound of formula I, or a pharmaceutically acceptable salt thereof, to the mammal.

The invention also provides a compound of formula I, or a pharmaceutically acceptable salt thereof, for use in the prophylactic or therapeutic treatment of a disease or condition associated with pathologic JAK activation (e.g. a cancer, a hematologic malignancy or other malignancy).

The invention also provides a compound of formula I, or a pharmaceutically acceptable salt thereof for use in medical therapy (e.g. for use in treating a disease or condition associated with pathologic JAK activation such as cancer, a hematologic malignancy or other malignancy).

The invention also provides a compound of formula I or a pharmaceutically acceptable salt thereof for the manufacture of a medicament for the treatment of a disease or condition associated with pathologic JAK activation (e.g. a cancer, a hematologic malignancy or other malignancy) in a mammal (e.g. a human).

The invention also provides a method for suppressing an immune response in a mammal (e.g. a human), comprising administering a compound of formula I, or a pharmaceutically acceptable salt thereof, to the mammal.

The invention also provides a compound of formula I, or a pharmaceutically acceptable salt thereof, for use in the prophylactic or therapeutic suppression of an immune response.

The invention also provides the use of a compound of formula I, or a pharmaceutically acceptable salt thereof for the manufacture of a medicament for suppressing an immune response in a mammal (e.g. a human).

The invention also provides processes and intermediates disclosed herein that are useful for preparing compounds of formula I or salts thereof.

### DETAILED DESCRIPTION OF THE INVENTION

### Definitions

The term “alkyl” as used herein refers to alkyl groups having from 1 to 10 carbon atoms which are straight or branched groups.

The term (C₁-C₈)alkyl as used herein refers to alkyl groups having from 1 to 8 carbon atoms which are straight or branched groups. This term is exemplified by groups such as methyl, ethyl, n-propyl, iso-propyl, n-butyl, t-butyl, isobutyl, n-pentyl, neopentyl, n-hexyl, n-heptyl and the like. The term (C₁-C₆)alkyl as used herein refers to alkyl groups having from 1 to 6 carbon atoms which are straight or branched groups.

The terms “alkenyl” or “alkene” as used herein refers to an alkenyl group having from 2 to 8 carbon atoms (i.e. (C₂-C₈)alkenyl) which are straight or branched groups and having at least one double bond. Such groups are exemplified by vinyl(ethen-1-yl), allyl, 1-propenyl, 2-propenyl(allyl), 1-methylethen-1-yl, 1-buten-1-yl, 2-buten-1-yl, 3-buten-1-yl, 1-methyl- 1-propen-1-yl, 2-methyl-1-propen-1-yl, 1-methyl-2-propen-1-yl, and 2-methyl-2-propen-1-yl, preferably 1-methyl-2-propen-1-yl and the like.

The term “alkynyl” or “alkyne” as used herein refers to an alkynyl group having from 2-8 carbon atoms (i.e. (C₂-C₈)alkynyl) which are straight or branched groups and having at least one triple bond. Such groups are exemplified by, but not limited to ethyn-1-yl, propyn-1-yl, propyn-2-yl, 1-methylprop-2-yn-1-yl, butyn-1-yl, butyn-2-yl, butyn-3-yl, and the like.

The term “halogen” as used herein refers to fluoro, chloro, bromo and iodo. In one embodiment halogen is preferably fluoro.

The term “cycloalkyl” as used herein refers to a saturated or partially unsaturated cyclic hydrocarbon ring systems, such as those containing 1 to 3 rings and 3 to 8 carbons per ring wherein multiple ring cycloalkyls can have, for example fused and Spiro bonds to one another. Exemplary groups include but are not limited to cyclopropyl, cyclobutyl, cyclopentyl, cyclohexyl, cyclohexenyl, cycloheptyl, cyclooctyl, cyclobutenyl, cyclohexenyl, cyclooctadienyl, decahydronaphthalene and spiro[4.5]decane.

The term “(C₃-C₈)cycloalkyl” as used herein refers to a cycloalkyl containing 3-8 carbon atoms. Exemplary groups include cyclopropyl, cyclobutyl, cyclopentyl, cyclohexyl and cyclooctyl. The term “(C₃-C₆)cycloalkyl” as used herein refers to a cycloalkyl containing 1 ring and 3-6 carbon atoms. Exemplary groups include cyclopropyl, cyclobutyl, cyclopentyl and cyclohexyl.

The term “aryl” as used herein refers to an aromatic cyclic group of from 6 to 14 carbon atoms having a single ring (e.g. phenyl) or multiple condensed rings (e.g. naphthyl or anthryl) wherein the condensed rings may be aromatic, saturated or partially saturated provided that at least one of the condensed rings is aromatic. Such multiple condensed rings may be optionally substituted with one or two oxo groups on the unsaturated or partially unsaturated ring portions of the multiple condensed ring. Exemplary aryls include, but are not limited to phenyl, indanyl naphthyl, 1,2-dihydronaphthyl and 1,2,3,4-tetrahydronaphthyl.

The term “heteroaryl” as used herein refers to a single aromatic ring of from about 1 to 6 carbon atoms and about 1-4 heteroatoms selected from the group consisting of oxygen, nitrogen and sulfur in the rings. The sulfur and nitrogen atoms may also be present in their oxidized forms. Such rings include but are not limited to pyridyl, pyrimidinyl, oxazolyl or furyl. The term heteroaryl also includes multiple condensed ring systems wherein a heteroaryl group (as defined above) can be fused with another heteroaryl (e.g. naphthyridinyl), a cycloalkyl (e.g. 5,6,7,8-tetrahydroquinolyl), an aryl (e.g. indazolyl) or a heterocycle (1,2,3,4-tetrahydronaphthyridine) to form a multiple condensed ring. Such multiple condensed rings may be optionally substituted with one or two oxo groups on the cycloalkyl or heterocycle portions of the condensed ring. Exemplary heteroaryls include but are not limited to pyridyl, pyrrolyl, pyrazinyl, pyrimidinyl, pyridazinyl, pyrazolyl, thienyl, indolyl, thiophenyl, imidazolyl, oxazolyl, thiazolyl, furyl, oxadiazolyl, thiadiazolyl, quinolyl, isoquinolyl, benzothiazolyl, benzoxazolyl, indazolyl, indolyl, quinoxalyl, quinazolyl, 5,6,7,8-tetrahydroisoquinoline and 4,5,6,7-tetrahydroindolyl.

The term “heterocycle” or “heterocyclic” or “heterocycloalkyl” as used herein refers to a single saturated or partially unsaturated ring (e.g. 3, 4, 5, 6, 7 or 8-membered ring) from about 1 to 7 carbon atoms and from about 1 to 3 heteroatoms selected from the group consisting of oxygen, nitrogen and sulfur in the rings. The sulfur and nitrogen atoms may also be present in their oxidized forms. Such rings include but are not limited to azetidinyl, tetrahydrofuranyl or piperidinyl. The term heterocycle also includes multiple condensed ring systems wherein a heterocycle group (as defined above) can be fused with another heterocycle (e.g. decahydronapthyridinyl), a cycloalkyl (e.g. decahydroquinolyl) or an aryl (e.g. 1,2,3,4-tetrahydroisoquinolyl) to form a multiple condensed ring. Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, tetrahydrothiophenyl, dihydrooxazolyl, tetrahydropyranyl, tetrahydrothiopyranyl, 1,2,3,4-tetrahydroquinolyl, benzoxazinyl and dihydrooxazolyl.

The term “cyclic amino” as used herein is a subgroup of heterocycles and refers to a 3-membered to 8-membered saturated or partially unsaturated, single ring which has at least one nitrogen atom, and may have one or more identical or different hetero atoms selected from the group consisting of nitrogen, oxygen, and sulfur wherein the nitrogen or sulfur atoms may be oxidized. Cyclic amino includes but is not limited to values such as aziridino, azetidino, pyrrolidino, piperidino, homopiperidino, morpholino, thiomorpholino, and piperazino.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

In cases where compounds are sufficiently basic or acidic, a salt of a compound of formula I can be useful as an intermediate for isolating or purifying a compound of formula I. Additionally, administration of a compound of formula I as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartrate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Specific values listed below for radicals, substituents, and ranges, are for illustration only; they do not exclude other defined values or other values within defined ranges for the radicals and substituents. The specific values listed below are specific values for compounds of formula I. The specific values listed below are also specific values for compounds of formula Ia, Ib, Ic, Id, Ie, If, Ig, Ih, Ii, Ij or Ik.

A specific group of compounds of formula I are compounds of formula Ia:

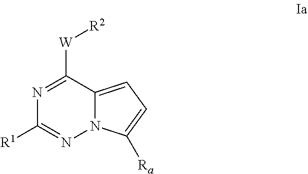

or a salt thereof.

Another specific group of compounds of formula I are compounds of formula Ib:

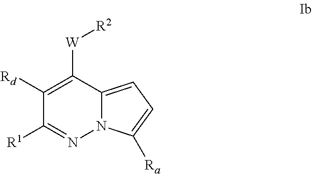

or a salt thereof.

Another specific group of compounds of formula I are compounds of formula Ic:

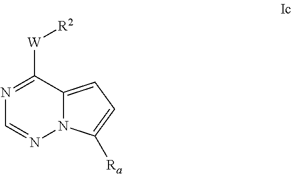

wherein W is heteroaryl or a salt thereof

Another specific group of compounds of formula I are compounds of formula Id:

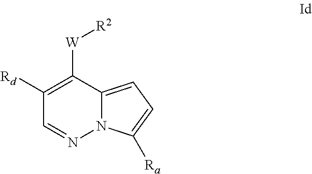

wherein W is heteroaryl or a salt thereof

Another specific group of compounds of formula I are compounds of formula Ie:

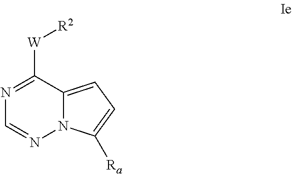

wherein Rₐ is H, NH₂, NO₂ or OH or a salt thereof

Another specific group of compounds of formula I are compounds of formula If:

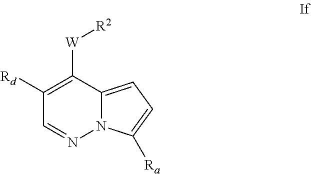

wherein Rₐ is H, NH₂, NO₂ or OH and R_{d} is H or —C(O)NH₂ or a salt thereof.

Another specific group of compounds of formula I are compounds of formula Ig:

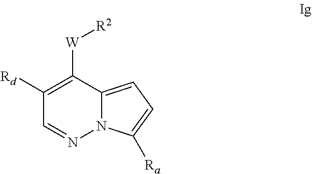

wherein Rₐ is H, NH₂, NO₂ or OH and R_{d} is H, CN or —C(O)NH₂ or a salt thereof.

Another specific group of compounds of formula I are compounds of formula Ih:

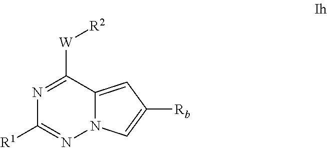

wherein R_{b} is H, NH₂, NO₂ or OH and R¹ is H, —C(O)NR_{f}R_{g}, —NR_{f}R_{g} or —NRₕC(O)ORₑ, or a salt thereof.

Another specific group of compounds of formula I are compounds of formula Ii:

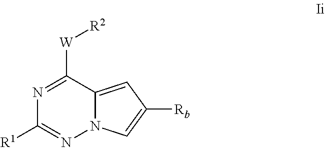

wherein R_{b} is H, NH₂, NO₂ or OH and R^{I} is H, —C(O)NH₂, —NH₂, —NHCO₂CH₃ or NHCO₂H or a salt thereof.

Another specific group of compounds of formula I are compounds of formula Ij:

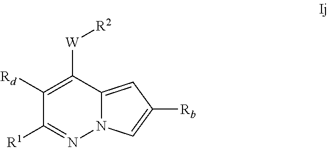

wherein R_{b} is H, NH₂, NO₂ or OH and R¹ is H, —C(O)NR_{f}R_{g}, —NR_{f}R_{g} or —NRₕC(O)ORₑ, or a salt thereof.

Another specific group of compounds of formula I are compounds of formula Ik:

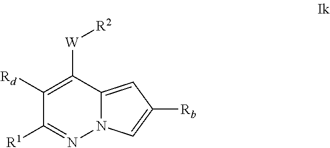

wherein R_{b} is H, NH₂, NO₂ or OH, R_{d} is H, CN or —C(O)NH₂ and R^{I} is H, —C(O)NH₂, —NH₂ or —NHCO₂CH₃ or a salt thereof.

A specific value for X is CRₐ.

A specific value for Rₐ is H.

Another specific value for Rₐ is —NRₙRₒ.

Another specific value for Rₐ is —NH₂.

Another specific value for Rₐ is H, NO₂ or —NRₙRₒ.

Another specific value for Rₐ is H or —NH₂.

Another specific value for Rₐ is H, NO₂, CO₂H, CO₂Rₙ₁, —C(O)NRₙRₒ, —C(O)NHNRₙRₒ, —C(O)NHNHCO₂Rₙ₁, —NHS(O)₂Rₙ₁, —NHCORₙ₂ or —NRₙRₒ.

Another specific value for Rₐ is H, NO₂, CO₂H, CO₂CH₂CH₃, —C(O)NH₂, —C(O)NHNH₂, —C(O)NHNHCO₂tBu, —NHS(O)₂CH₃, —NHCOCF₃, —NH₂ or —NHCH₂CO₂H.

Another specific value for X is N.

A specific value for Y is CR_{b}.

A specific value for R_{b} is H.

Another specific value for R_{b} is H, NH₂, NO₂ or OH.

Another specific value for R_{b} is H, NO₂, CO₂H, —NHS(O)₂Rₙ₁, —NHCORₙ₂ or —NRₙRₒ.

Another specific value for R_{b} is H, NO₂, CO₂H, —NHS(O)₂CH₃, —NHCOCF₃, —NH₂ or —NHCH₂CO₂H.

Another specific value for R_{b} is H or NO₂.

Another specific value for Y is N.

A specific value for Z is CR_{c}.

A specific value for Rₑ is H.

Another specific value for Z is N.

A specific value for Y is CR_{d}.

A specific value for R_{d} is H, heteroaryl or —C(O)NRᵣRₛ.

Another specific value for R_{d} is H or —C(O)NRᵣRₛ.

Another specific value for R_{d} is —C(O)NH₂.

Another specific value for R_{d} is H, CN or —C(O)NRᵣRₛ.

Another specific value for R_{d} is heteroaryl substituted with —NH₂ or —CH₂OH.

Another specific value for R_{d} is:

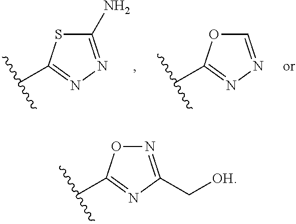 [SMILES: *c1nnc(N)s1 ; *c1nnco1 ; *c1nc(CO)no1]

A specific group of compounds of formula I are compounds wherein Rᵣ and Rₛ are H.

Another specific value for Y is N.

Another specific group of compounds of formula I are compounds wherein X and Y are N.

Another specific group of compounds of formula I are compounds wherein X and Z are N.

Another specific group of compounds of formula I are compounds wherein X and V are N.

Another specific group of compounds of formula I are compounds wherein Y and Z are N.

Another specific group of compounds of formula I are compounds wherein Y and V are N.

Another specific group of compounds of formula I are compounds wherein Z and V are N.

Another specific group of compounds of formula I are compounds wherein Y and Z are CH.

Another specific group of compounds of formula I are compounds wherein X, Y and Z are CH.

Another specific group of compounds of formula I are compounds wherein X is CRₐ, Y is CR_{b} and Z is CR_{c}.

A specific value for R¹ is H, —C(O)NR_{f}R_{g}, —NR_{f}R_{g} or —NRₕC(O)ORₑ.

Another specific value for R¹ is H, —NR_{f}R_{g} or —NRₕC(O)ORₑ.

Another specific value for R¹ is H, —NH₂ or —NHC(O)OCH₃.

Another specific value for R¹ is H, —NR_{f}R_{g}, —NR_{b}C(O)ORₑ or —NRₕS(O)₂Rₑ.

Another specific value for R¹ is H, —NH₂, —NHC(O)OCH₃, —NHCH₂C(O)OH, —NHCH₂CH₂C(O)OH, —NHCH(CO₂H)CH₂OH, —NHCH(CO₂H)₂, or —NHS(O)₂CH₃.

A specific value for W is heterocycle.

Another specific value for W is piperidinyl, 4-methylpiperidinyl, 3-methylpiperidinyl, 3-fluoropiperidinyl, 4-fluoropiperidinyl, chromanyl, benzooxetanyl, dihydrobenzothiazinyl or dihydrobenzoxazinyl.

Another specific value for W is aryl.

Another specific value for W is phenyl or benzocyclobutyl.

Another specific value for W is heteroaryl.

Another specific value for W is pyrrolyl, thienyl, benzothienyl, furyl, benzofuranyl, thiazolyl, oxazolyl, pyrazolyl, imidazolyl, indolyl or oxadiazolyl.

Another specific value for W is heterocycle, wherein heterocycle may be optionally substituted with one or more groups selected from R_{w} and oxo.

Another specific value for W is piperidinyl, 4-methylpiperidinyl, 3-methylpiperidinyl, 3-fluoropiperidinyl, 4-fluoropiperidinyl chromanyl, benzooxetanyl, dihydrobenzothiazinyl or dihydrobenzoxazinyl, wherein piperidinyl, 4-methylpiperidinyl, 3-methylpiperidinyl, 3-fluoropiperidinyl, 4-fluoropiperidinyl chromanyl, benzooxetanyl, dihydrobenzothiazinyl or dihydrobenzoxazinyl may be optionally substituted with one or more groups selected from R_{w} and oxo.

Another specific value for W is aryl, wherein aryl is optionally substituted with one or more R_{w} groups.

Another specific value for W is phenyl or benzocyclobutyl, wherein phenyl or benzocyclobutyl is optionally substituted with one or more R, groups.

Another specific value for W is heteroaryl, wherein heteroaryl is optionally substituted with one or more R_{w} groups.

Another specific value for W is pyrrolyl, thienyl, benzothienyl, furyl, benzofuranyl, thiazolyl, oxazolyl, pyrazolyl, imidazolyl, indolyl or oxadiazolyl, wherein pyrrolyl, thienyl, benzothienyl, furyl, benzofuranyl, thiazolyl, oxazolyl, pyrazolyl, imidazolyl, indolyl or oxadiazolyl is optionally substituted with one or more R_{w} groups.

Another specific value for W is pyrazolyl, wherein pyrazolyl is optionally substituted with one or more R_{w} groups.

Another specific value for W—R² is:

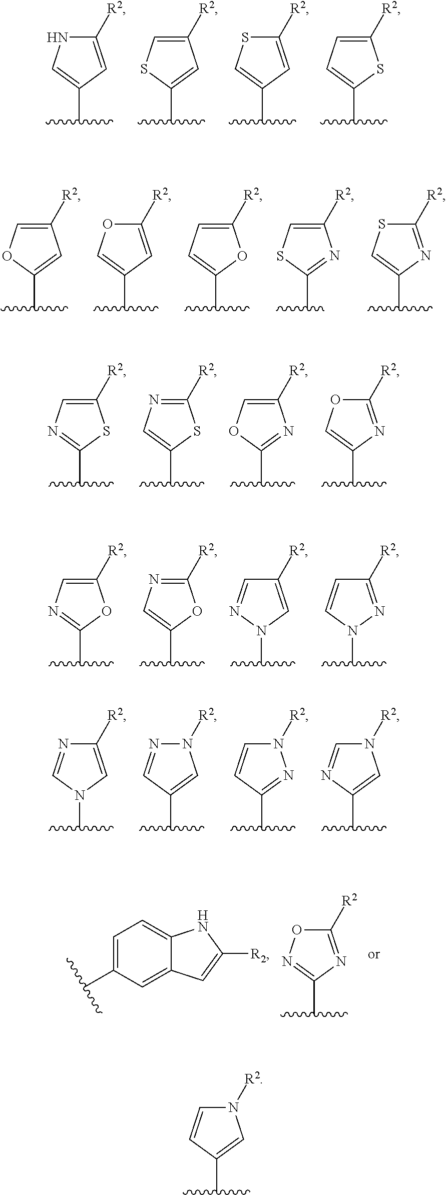

Another specific value for W—R² is:

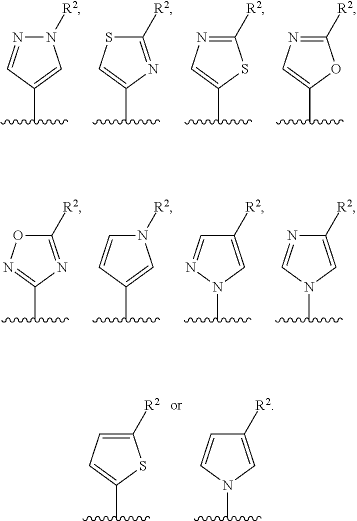

Another specific value for W—R² is:

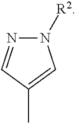

A specific group of compounds of formula I are compounds wherein R² is absent.

A specific value for R² is heteroaryl, heterocycle, —(C₁-C₆)alkyl, —S(O)₂NR_{z1}R_{z2}, —C(O)R_{z}, —C(O)NR_{z1}R_{z2} or —C(O)heteroaryl.

Another specific value for R² is:

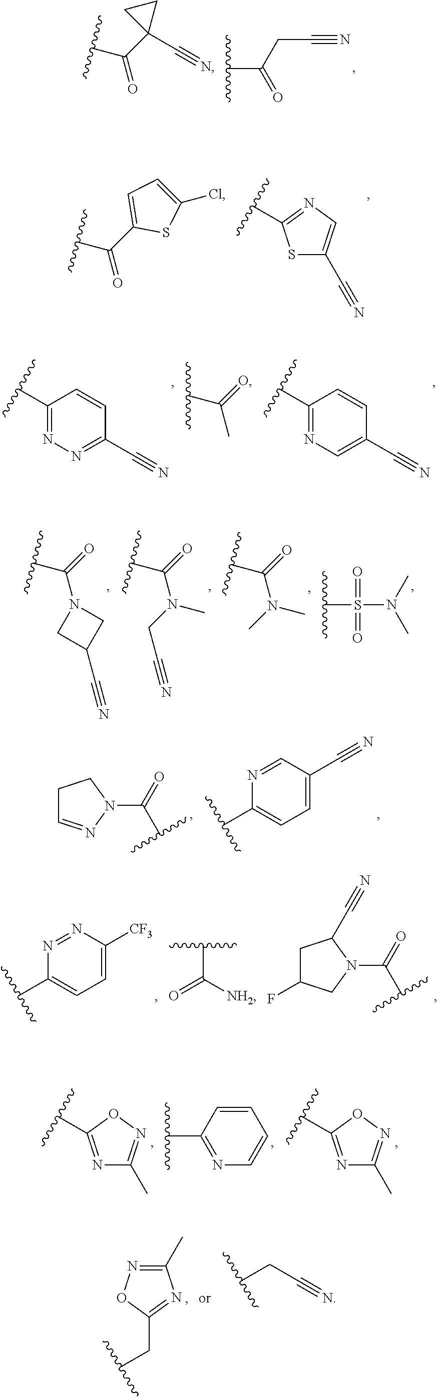

Another specific value for R² is —(C₁-C₆)alkyl, —OR_{z}, —Oheterocycle, or —Oheteroaryl. Another specific value for R² is:

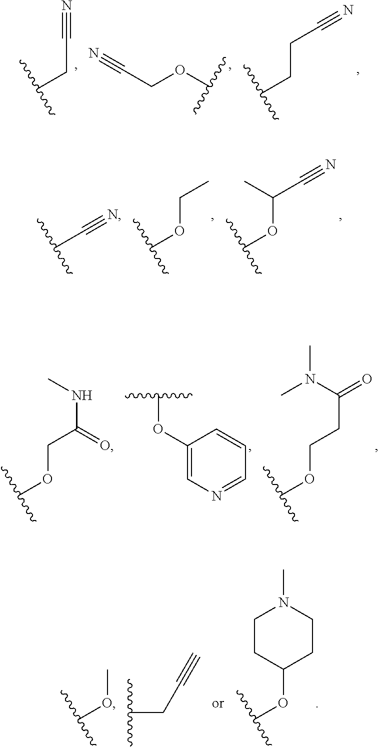

Another specific value for R² is heterocycle, (C₁-C₆)alkyl or (C₃-C₆)cycloalkyl.

Another specific value for R² is oxetanyl, tetrahydrofuranyl, oxiranyl, tetrahydropyranyl, azetidinyl, aziridinyl, piperidinyl, pyrrolidinyl, cyclopropyl, cyclobutyl, cyclopentyl, cyclohexyl, ethyl or propyl.

Another specific value for R² is heteroaryl, heterocycle, —(C₁-C₆)alkyl, —S(O)₂NR_{z1}R_{z2}, —C(O)R_{z}, —C(O)NR_{z1}R_{z2} or —C(O)heteroaryl, wherein any alkyl or heteroaryl of R² may be optionally substituted with one or more R_{y} groups and wherein any heterocycle of R² may be optionally substituted with one or more groups selected from oxo, ═CHCN and R_{y}.

Another specific value for R² is heteroaryl, heterocycle, —(C₁-C₆)alkyl, —S(O)₂NR_{z1}R_{z2}, —C(O)R_{z}, —C(O)NR_{z1}R_{z2} or —C(O)heteroaryl, wherein any —(C₁-C₆)alkyl, —C(O)heteroaryl or heteroaryl of R² may be optionally substituted with one or more R_{y} groups and wherein any heterocycle of R² may be optionally substituted with one or more groups selected from oxo, ═CHCN and R_{y}.

Another specific value for R² is —(C₁-C₈)alkyl, —OR_{z}, —Oheterocycle, or —Oheteroaryl, wherein any alkyl or heteroaryl of R² may be optionally substituted with one or more R_{y} groups and wherein any —Oheterocycle of R² may be optionally substituted with one or more groups selected from oxo, ═CHCN and R_{y}.

Another specific value for R² is —(C₁-C₈)alkyl, —OR_{z}, —Oheterocycle, or —Oheteroaryl, wherein any —(C₁-C₈)alkyl or —Oheteroaryl of R² may be optionally substituted with one or more R_{y} groups and wherein any —Oheterocycle of R² may be optionally substituted with one or more groups selected from oxo, ═CHCN and R_{y}.

Another specific value for R² is heterocycle, (C₁-C₈)alkyl or (C₃-C₈)cycloalkyl, wherein any (C₁-C₈)alkyl of R² may be optionally substituted with one or more R_{y} groups, and wherein (C₃-C₈)cycloalkyl of R² may be optionally substituted with one or more groups selected from oxo, ═CHCN and R_{y}.

Another specific value for R² is oxetanyl, tetrahydrofuranyl, oxiranyl, tetrahydropyranyl, azetidinyl, aziridinyl, piperidinyl, pyrrolidinyl, cyclopropyl, cyclobutyl, cyclopentyl, cyclohexyl, ethyl or propyl, wherein any ethyl or propyl of R² may be optionally substituted with one or more R_{y} groups and wherein oxetanyl, tetrahydrofuranyl, oxiranyl, tetrahydropyranyl, azetidinyl, aziridinyl, piperidinyl, pyrrolidinyl, cyclopropyl, cyclobutyl, cyclopentyl, or cyclohexyl of R² may be optionally substituted with one or more groups selected from oxo, ═CHCN and R_{y}.

Another specific value for R² is —(C₁-C₈)alkyl, wherein —(C₁-C₈)alkyl may be optionally substituted with one or more R_{y} groups.

Another specific group of compounds of formula I are compounds wherein R² is substituted with one or more R_{y} groups.

A specific value for R_{y} is R_{z}, OH, CN, OR_{z}, —Oheteroaryl, —OC(O)R_{z}, —S(O)₂R_{z}, —OS(O)₂R_{z}, —S(O)₂aryl, —OS(O)₂aryl, —S(O)₂heteroaryl, —OS(O)₂heteroaryl, —C(O)R_{z}, —C(O)aryl, —OC(O)aryl, —C(O)heteroaryl, —OC(O)heteroaryl, aryl, heterocycle or heteroaryl wherein any aryl or heteroaryl of R_{y} is optionally substituted with one or more halogen, (C₁-C₃)alkyl, CF₃, —O(C₁-C₃)alkyl, CN, —OCH₂CN, NR_{z1}R_{z2}, —NO₂, —CHO, —Oaryl, —OCF₃, —C(O)OR_{z}, —C(O)OH, aryl, —NHCOR_{z}, —NHS(O)₂R_{z}, —C(O)NR_{z1}R_{z2}, —NHCONR_{z1}R_{z2}, —NHCOheteroaryl, —NHC(O)OR_{z}, —(C₂-C₆)alkynyl, —Saryl or heteroaryl wherein heteroaryl is optionally substituted with (C₁-C₃)alkyl and wherein any heterocycle of R_{y} is optionally substituted with one or more R_{z}, —S(O)₂R_{z}, —S(O)₂aryl, —S(O)₂heteroaryl, —C(O)R_{z}, —C(O)aryl, —C(O)heteroaryl or heteroaryl wherein aryl or heteroaryl is optionally substituted with one or more halogen or (C₁-C₃)alkyl.

A specific value for R_{y} is R_{z}, OH, CN, OR_{z}, —Oheteroaryl, —OC(O)R_{z}, —S(O)₂R_{z}, —OS(O)₂R_{z}, —S(O)₂aryl, —OS(O)₂aryl, —S(O)₂heteroaryl, —OS(O)₂heteroaryl, —C(O)R_{z}, —C(O)aryl, —OC(O)aryl, —C(O)heteroaryl, —OC(O)heteroaryl, aryl, heterocycle or heteroaryl wherein any aryl, Oheteroaryl, —S(O)₂aryl, —OS(O)₂aryl, —S(O)₂heteroaryl, —OS(O)₂heteroaryl, —C(O)aryl, —OC(O)aryl, —C(O)heteroaryl, —OC(O)heteroaryl or heteroaryl of R_{y} is optionally substituted with one or more halogen, R_{z}, —OR_{z}, CN, NR_{z1}R_{z2}, —NO₂, —CHO, —Oaryl, —C(O)OR_{z}, —C(O)OH, aryl, —NHCOR_{z}, —NHS(O)₂R_{z}, —C(O)NR_{z1}R_{z2}, —NHCONR_{z1}R_{z2}, —NHCOheteroaryl, —NHC(O)OR_{z}, —(C₂-C₆)alkynyl, —Saryl or heteroaryl wherein heteroaryl or —NHCOheteroaryl is optionally substituted with (C₁-C₃)alkyl, and wherein any heterocycle of R_{y} is optionally substituted with one or more R_{z}, —S(O)₂R_{z}, —S(O)₂aryl, —S(O)₂heteroaryl, —C(O)R_{z}, —C(O)aryl, —C(O)heteroaryl or heteroaryl wherein aryl or heteroaryl is optionally substituted with one or more halogen or (C₁-C₃)alkyl.

Another specific value for R² is:

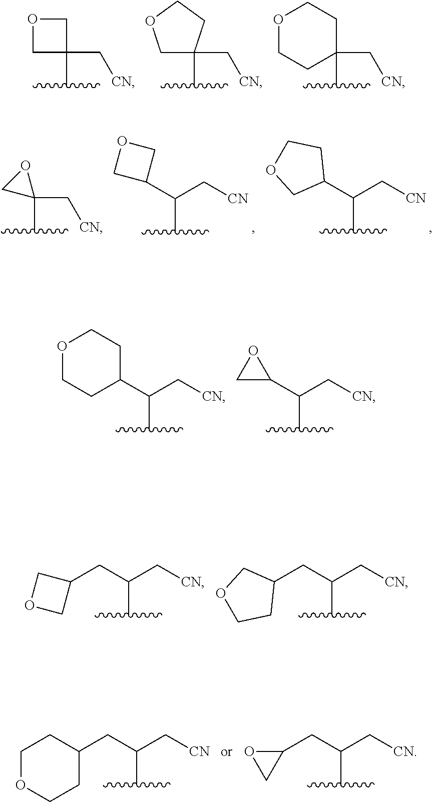

Another specific value for R² is:

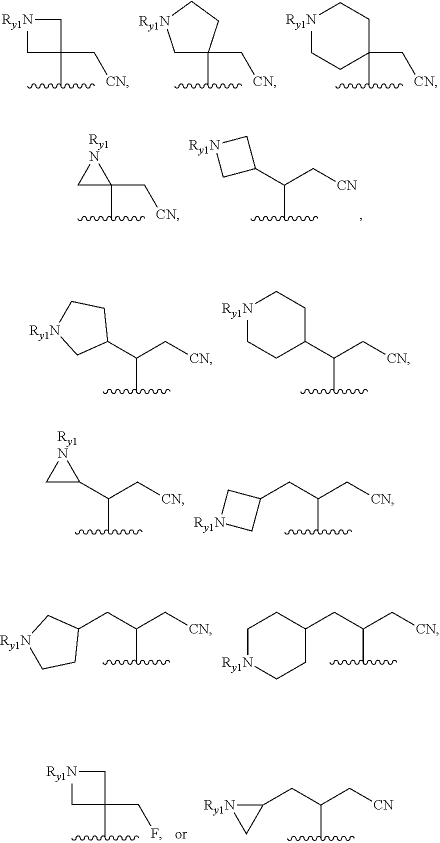

wherein each R_{y1} is independently H, R_{z}, —S(O)₂R_{z}, —S(O)₂aryl, —S(O)₂heteroaryl, —C(O)R_{z}, —C(O)aryl, —C(O)heteroaryl, or heteroaryl wherein any aryl or heteroaryl of R_{y1} is optionally substituted with one or more halogen or (C₁-C₃)alkyl.

Another specific value for R² is:

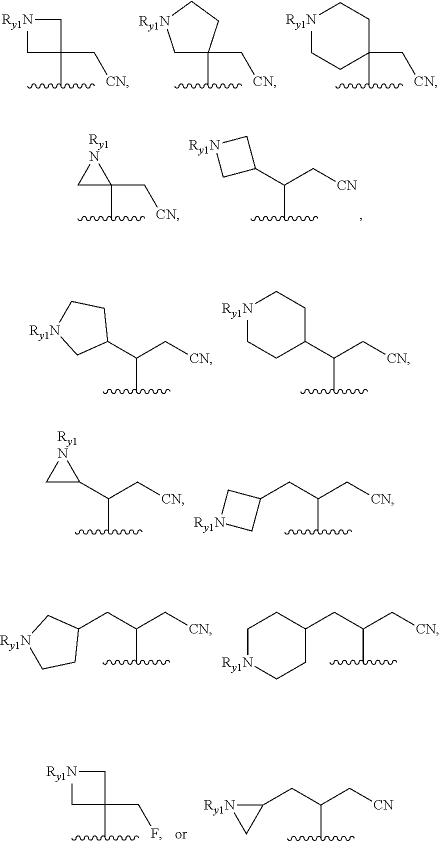

wherein each R_{y1} is independently H, R_{z}, —S(O)₂R_{z}, —S(O)₂aryl, —S(O)₂heteroaryl, —C(O)R_{z}, —C(O)aryl, —C(O)heteroaryl, or heteroaryl wherein any —S(O)₂aryl, —S(O)₂heteroaryl, —C(O)aryl, —C(O)heteroaryl or heteroaryl of R_{y1} is optionally substituted with one or more halogen or (C₁-C₃)alkyl.

Another specific value for R² is:

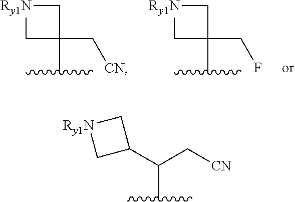

wherein each R_{y1} is independently H, R_{z}, —S(O)₂R_{z}, —S(O)₂aryl, —S(O)₂heteroaryl, —C(O)R_{z}, —C(O)aryl, —C(O)heteroaryl, or heteroaryl wherein any aryl or heteroaryl of R_{y1} is optionally substituted with one or more halogen or (C₁-C₃)alkyl.

Another specific value for R² is:

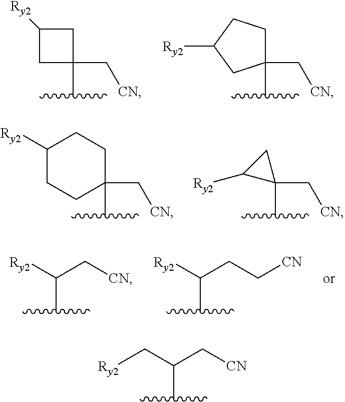

wherein each R_{y2} is independently H or R_{y}.

Another specific value for R² is:

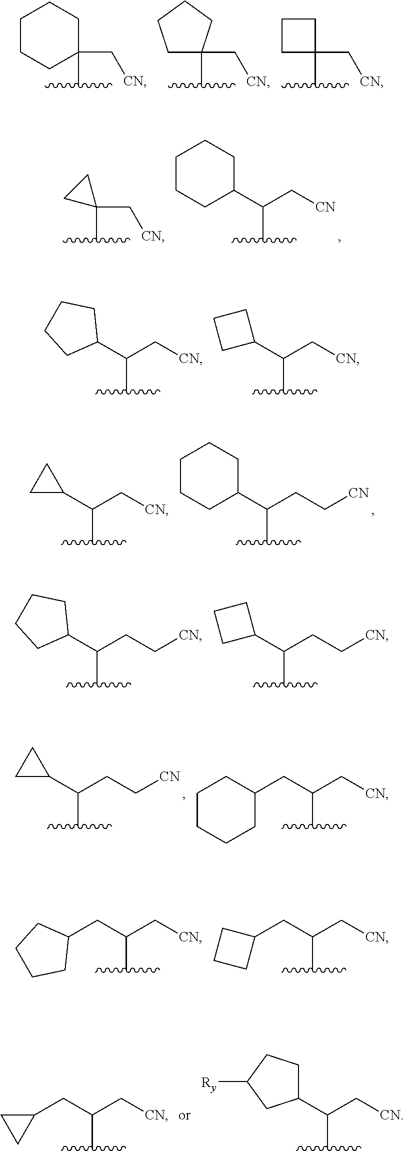

Another specific value for R² is:

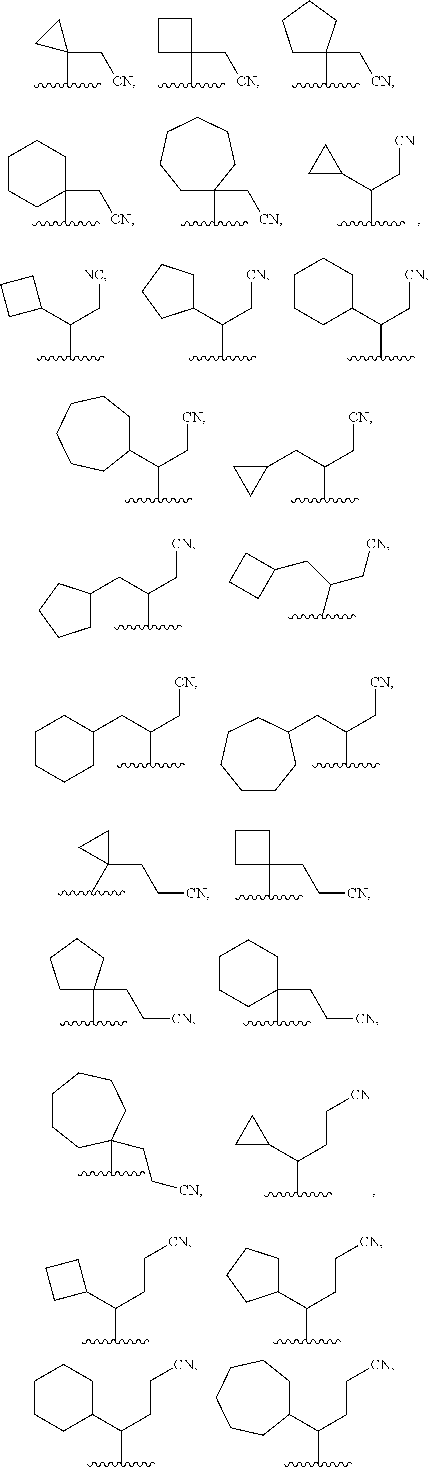

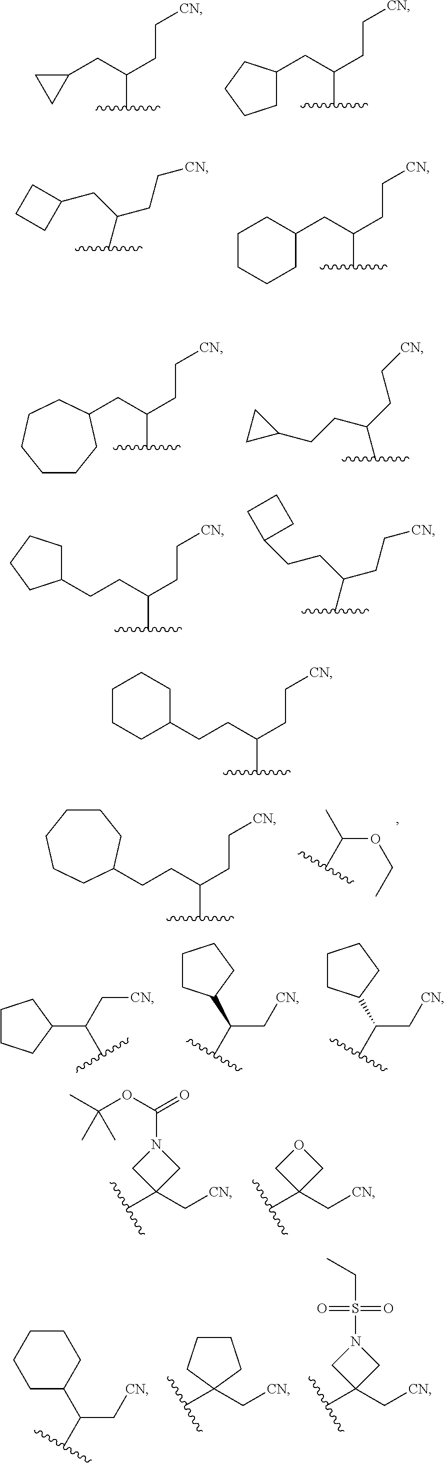

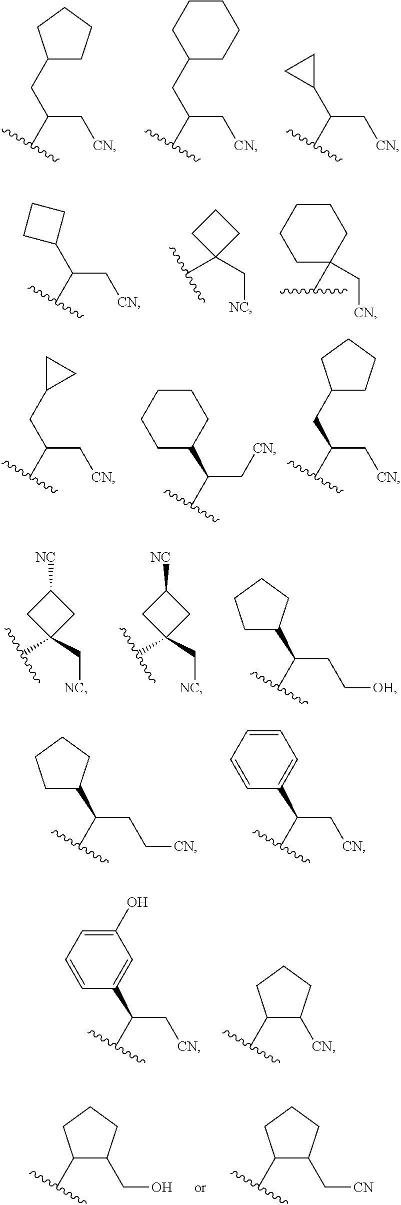

Another specific value for R_{y} is NR_{z1}R_{z2} or NHCOR_{z}.

Another specific value for R_{y} is —NH₂, —NHC(O)(C₁-C₄)alkyl or —NHCO(C₃-C₆)cycloalkyl.

Another specific value for R_{y} is R_{z}, CN or OR_{z}.

Another specific value for R² is —(C₁-C₈)alkyl, wherein —(C₁-C₈)alkyl may be optionally substituted with one or more groups selected from R_{z}, CN or OR_{z}.

Another specific value for R² is —(C₁-C₈)alkyl, wherein —(C₁-C₈)alkyl may be optionally substituted with one or more groups selected from cyclopentyl, CN and ethoxy.

Another specific value for R_{y} is R_{z}, CN, OR_{z}, —Oheteroaryl, —OC(O)R_{z}, —S(O)₂R_{z}, —OS(O)₂R_{z}, —S(O)₂aryl, —OS(O)₂aryl, —S(O)₂heteroaryl, —OS(O)₂heteroaryl, —C(O)R_{z}, —C(O)aryl, —OC(O)aryl, —C(O)heteroaryl, —OC(O)heteroaryl, or heteroaryl wherein any aryl or heteroaryl of R_{y} is optionally substituted with one or more halogen or (C₁-C₃)alkyl.

Another specific value for R_{y} is R_{z}, CN, OR_{z}, —Oheteroaryl, —OC(O)R_{z}, —S(O)₂R_{z}, —OS(O)₂R_{z}, —S(O)₂aryl, —OS(O)₂aryl, —S(O)₂heteroaryl, —OS(O)₂heteroaryl, —C(O)R_{z}, —C(O)aryl, —OC(O)aryl, —C(O)heteroaryl, —OC(O)heteroaryl, or heteroaryl wherein any Oheteroaryl, —S(O)₂aryl, —OS(O)₂aryl, —S(O)₂heteroaryl, —OS(O)₂heteroaryl, —C(O)aryl, —OC(O)aryl, —C(O)heteroaryl, —OC(O)heteroaryl or heteroaryl of R_{y} is optionally substituted with one or more halogen or (C₁-C₃)alkyl.

Another specific value for R_{y} is OH, CN, —CO₂R_{z}, aryl or heteroaryl wherein any aryl or heteroaryl of R_{y} is optionally substituted with one or more halogen, (C₁-C₃)alkyl, CF₃, —O(C₁-C₃)alkyl, CN, —OCH₂CN, NR_{z1}R_{z2}, —NO₂, —CHO, —Oaryl, —OCF₃, —C(O)OR_{z}, —C(O)OH, aryl, —NHCOR_{z}, —NHS(O)₂R_{z}, —C(O)NR_{z1}R_{z2}, —NHCONR_{z1}R_{z2}, —NHCOheteroaryl, —NHC(O)OR_{z}, —(C₂-C₆)alkynyl, —Saryl or heteroaryl wherein heteroaryl is optionally substituted with (C₁-C₃)alkyl.

Another specific value for R_{y} is OH, CN, —CO₂R_{z}, aryl or heteroaryl wherein any aryl or heteroaryl of R_{y} is optionally substituted with one or more halogen, (C₁-C₃)alkyl, CF₃, —O(C₁-C₃)alkyl, CN, —OCH₂CN, NR_{z1}R_{z2}, —NO₂, —CHO, —Oaryl, —OCF₃, —C(O)OR_{z}, —C(O)OH, aryl, —NHCOR_{z}, —NHS(O)₂R_{z}, —C(O)NR_{z1}R_{z2}, —NHCONR_{z1}R_{z2}, —NHCOheteroaryl, —NHC(O)OR_{z}, —(C₂-C₆)alkynyl, —Saryl or heteroaryl wherein heteroarylor —NHCOheteroaryl is optionally substituted with (C₁-C₃)alkyl.

Another specific value for R² is:

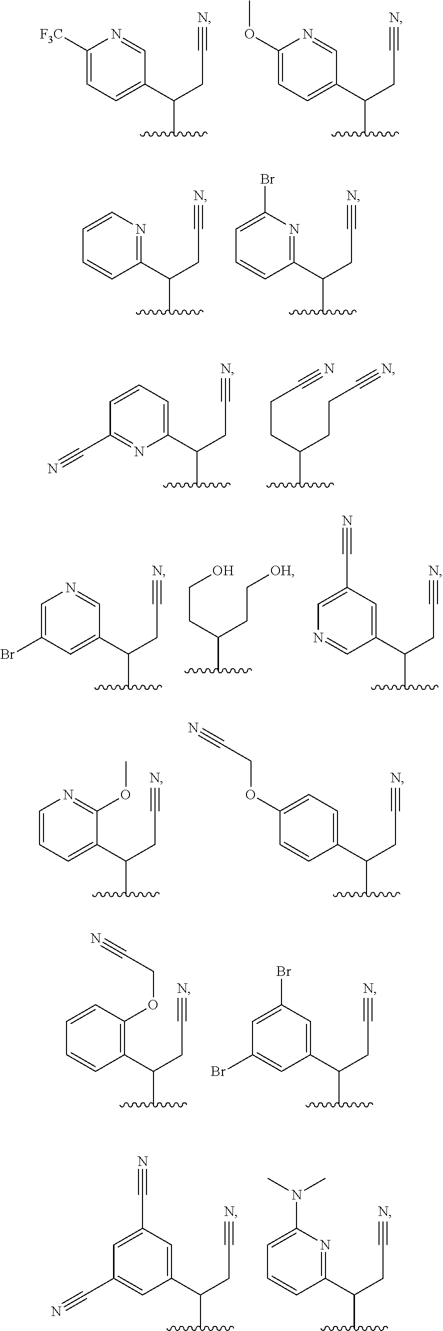

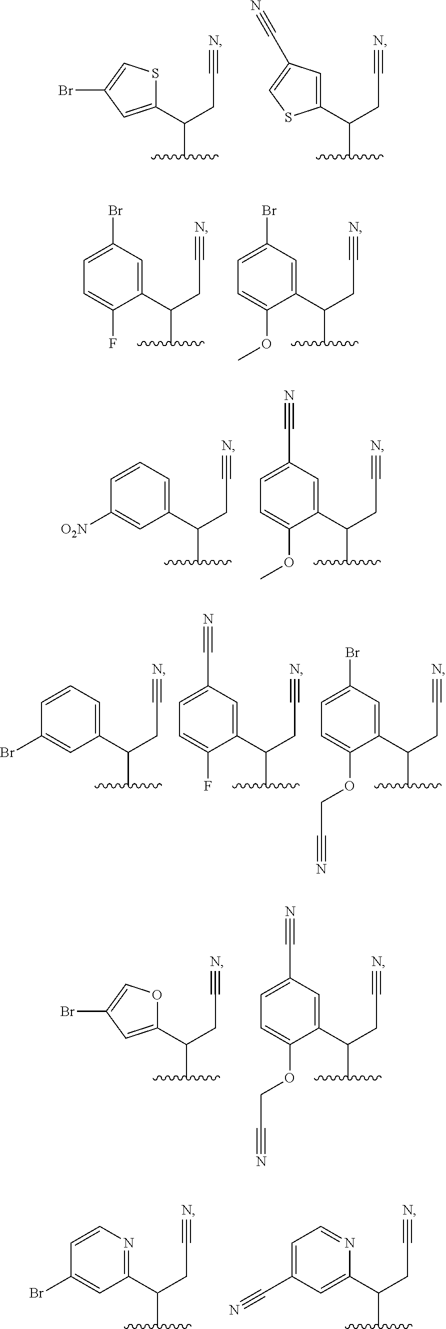

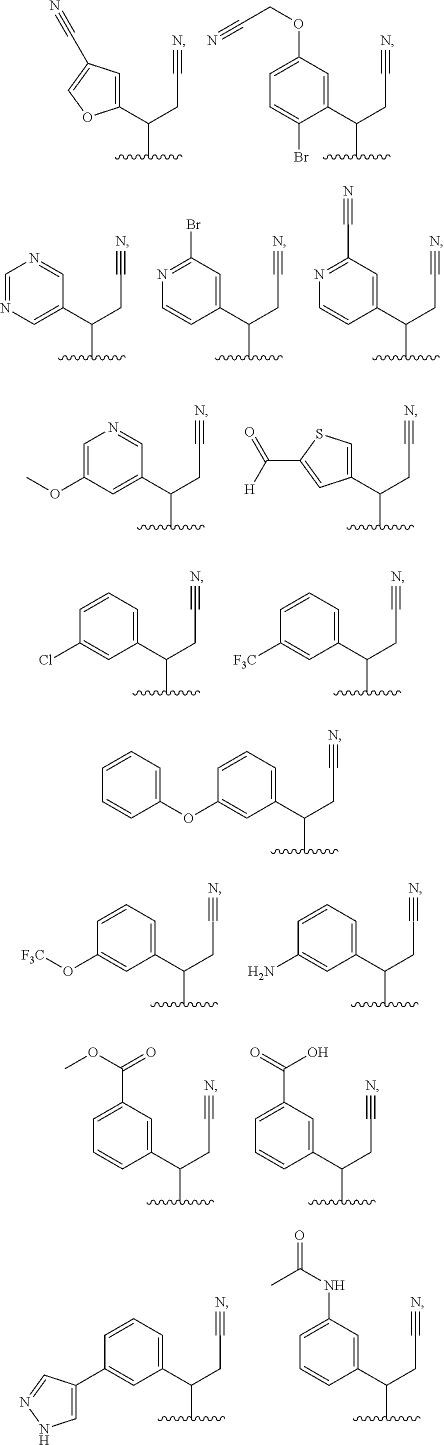

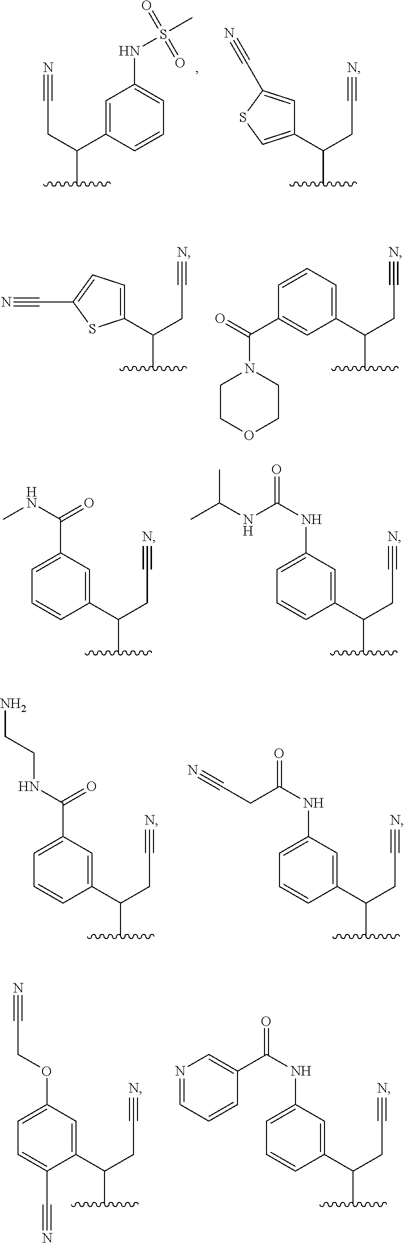

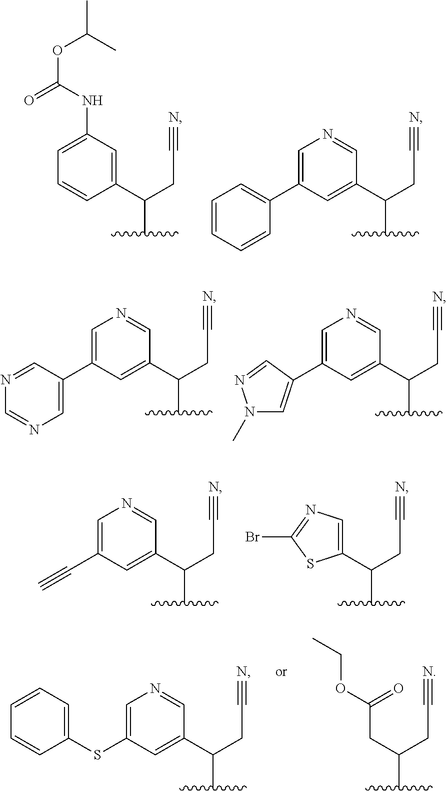

A specific value for W—R² is:

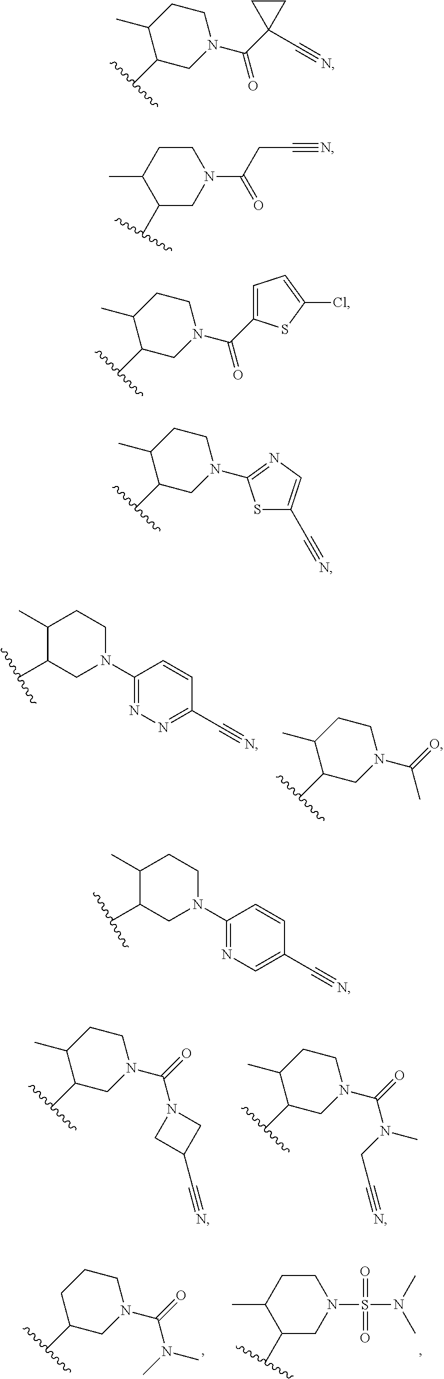

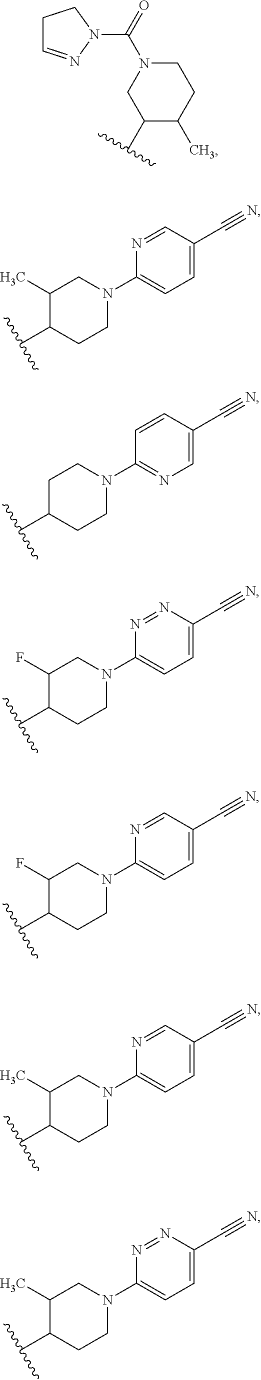

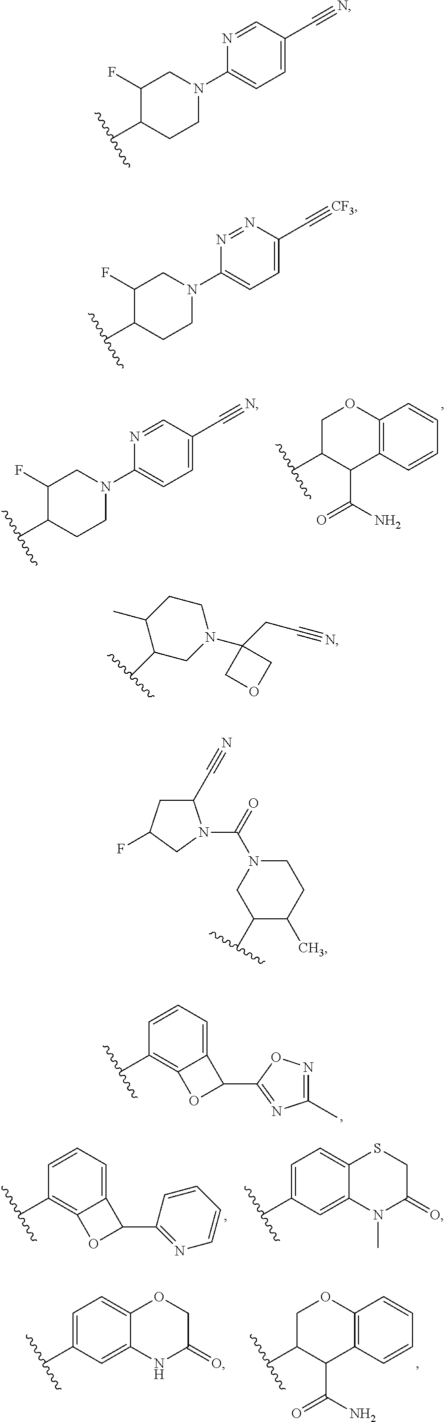

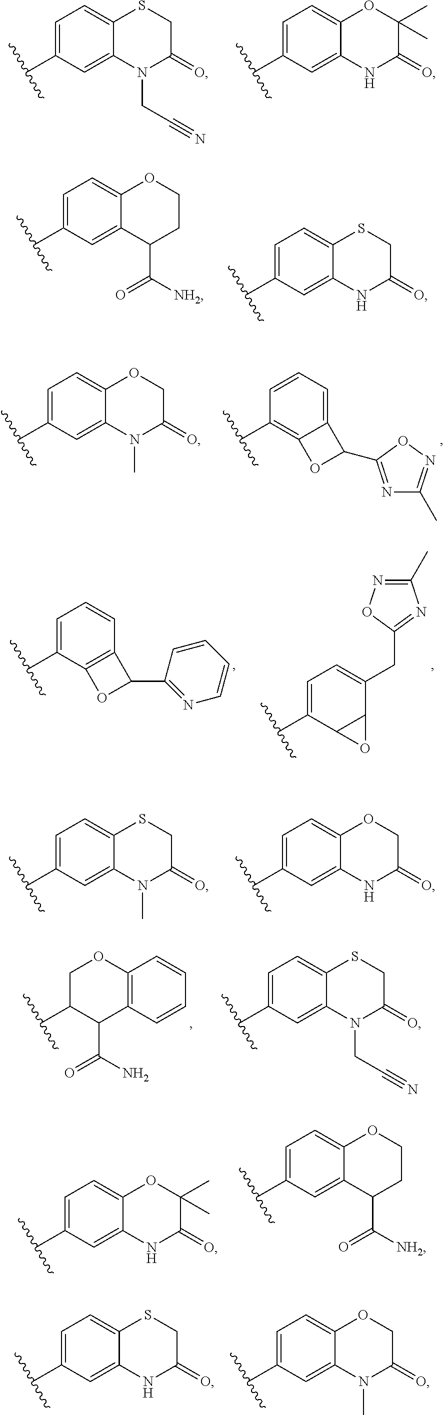

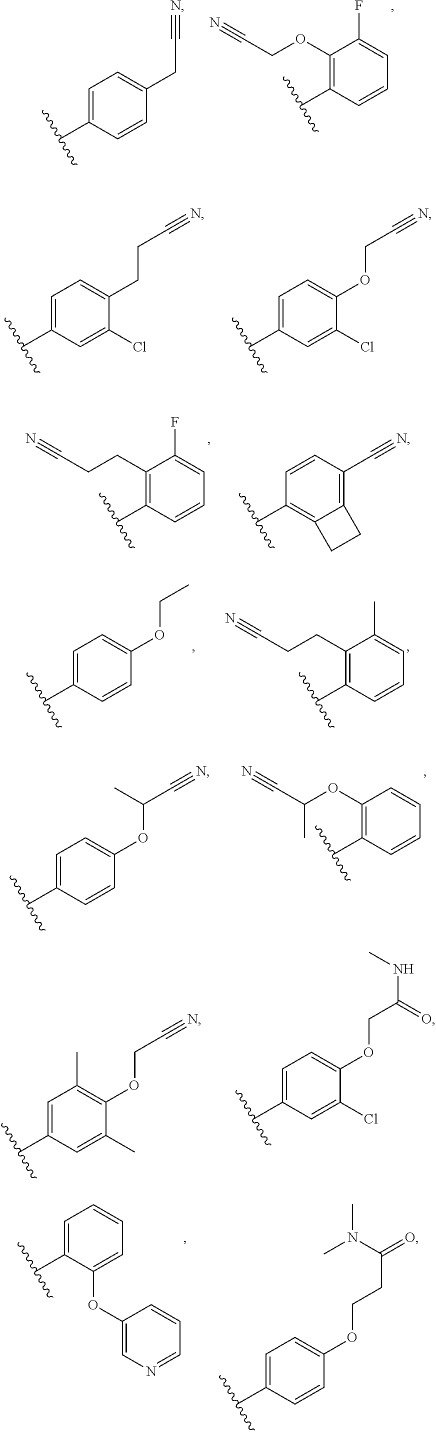

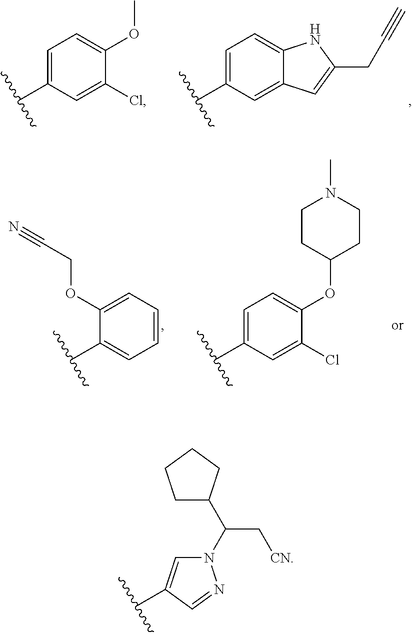

Another specific value for W—R² is:

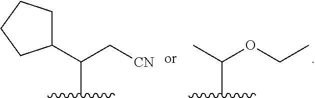

In one embodiment, the invention provides a compound of the invention which is a compound of formula I:

wherein:

W is heteroaryl, heterocycle or aryl, wherein any aryl or heteroaryl of W may be optionally substituted with one or more (e.g. 1, 2, 3, 4 or 5) R_{w} groups and wherein any heterocycle of W may be optionally substituted with one or more groups selected from R_{w} and oxo;

X is N or CRₐ; Y is N or CR_{b}; Z is N or CR_{c}; and V is N or CR_{d} provided that no more than two of X, Y, Z or V is N;

R¹ is H, halogen, —(C₁-C₈)alkyl, —(C₂-C₈)alkenyl, —(C₂-C₈)alkynyl, —(C₃-C₈)cycloalkyl, aryl, heteroaryl, heterocycle, NO₂, CN, —OH, —ORₑ, —NR_{f}R_{g}, N₃, SH, —SRₑ, —C(O)Rₕ, —C(O)ORₕ, —C(O)NR_{f}R_{g}, —C(═NRₕ)NR_{f}R_{g}, —NRₕCORₑ, —NRₕC(O)ORₑ, —NRₕS(O)₂Rₑ, —NRₕCONR_{f}R_{g}, —OC(O)NR_{f}R_{g}, —S(O)Rₑ, —S(O)NR_{f}R_{g}, —S(O)₂Rₑ, —S(O)₂OH, or —S(O)₂NR_{f}R_{g}, wherein any aryl or heteroaryl of R₁ may be optionally substituted with one or more (e.g. 1, 2, 3, 4 or 5) Rᵢ groups and wherein any alkyl, cycloalkyl, alkenyl, alkynyl or heterocycle of R¹ may be optionally substituted with one or more groups selected from Rᵢ, oxo and ═NORₕ;

R² is selected from halogen, aryl, heteroaryl, heterocycle, —(C₁-C₈)alkyl, —(C₂-C₈)alkenyl, —(C₂-C₈)alkynyl, —(C₃-C₈)cycloalkyl, OH, CN, —Oaryl, —Oheterocycle, —Oheteroaryl, —OC(O)R_{z}, —OC(O)NR_{z1}R_{z2}, SH, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, —CHO, —C(O)R_{z}, —C(O)OH, —C(O)OR_{z}, —C(O)NR_{z1}R_{z2}, —C(O)heteroaryl and —C(O)C(O)R_{z}, wherein any alkyl, alkenyl, alkynyl, aryl or heteroaryl of R² may be optionally substituted with one or more (e.g. 1, 2, 3, 4 or 5) R_{y} groups and wherein any heterocycle or cycloalkyl of R² may be optionally substituted with one or more (e.g. 1, 2, 3, 4 or 5) groups selected from oxo,=CHCN and R_{y}; or R² is absent;

Rₐ is H, OH, NO₂, CO₂H, —C(O)NR_{b}Rₒ, —NRₙRₒ, halogen or —(C₁-C₆)alkyl wherein alkyl is optionally substituted with one or more (e.g. 1, 2, 3, 4 or 5) Rₚ groups;

R_{b} is H, OH, NO₂, CO₂H, —NRₙRₒ, halogen or —(C₁-C₆)alkyl wherein alkyl is optionally substituted with one or more (e.g. 1, 2, 3, 4 or 5) Rₚ groups;

R_{c} is H, OH, NO₂, CO₂H, —NRₙRₒ, halogen or —(C₁-C₆)alkyl wherein alkyl is optionally substituted with one or more (e.g. 1, 2, 3, 4 or 5) Rₚ groups;

R_{d} is H, halogen, —(C₁-C₆)alkyl, —(C₂-C₆)alkenyl, —(C₂-C₆)alkynyl, —(C₃-C₆)cycloalkyl, aryl, heteroaryl, heterocycle, NO₂, CN, OH, —OR_{q}, —NRᵣRₛ, N₃, —SH, —SR_{q}, —C(O)(C₁-C₆)alkyl, —C(O) (C₂-C₆)alkenyl, 13 C(O)(C₂-C₆)alkynyl, —C(O)(C₃-C₆)cycloalkyl, —C(O)aryl, —C(O)heteroaryl, —C(O)heterocycle, —C(O)ORₜ, —C(O)NRᵣRₛ, —C(═NRₜ)NRᵣRₛ, —NRₜCOR_{q}, —NRₜC(O)OR_{q}, —NRₜS(O)₂R_{q}, —NRₜCONRᵣRₛ, —OC(O)NRᵣ, Rₛ, —S(O)R_{q}, —S(O)NRᵣSₛ, —S(O)₂R_{q}, —S(O)₂OH, —S(O)₂NRᵣRₛ or —C(═O)C(═O)NH(C₁-C₆)alkyl, wherein any aryl or heteroaryl of R_{d} may be optionally substituted with one or more (e.g. 1, 2, 3, 4 or 5) Rᵢ groups and wherein any alkyl, cycloalkyl, alkenyl, alkynyl or heterocycle of R_{d} may be optionally substituted with one or more (e.g. 1, 2, 3, 4 or 5) groups selected from R₁, oxo and =NORₜ;

Rₑ is alkyl, alkenyl, alkynyl, cycloalkyl, heterocycle, heteroaryl or aryl;

R_{f} and R_{g} are each independently selected from H, alkyl, alkenyl, alkynyl, cycloalkyl, heterocycle and heteroaryl; or R_{f} and R_{g} together with the nitrogen to which they are attached form a pyrrolidino, piperidino, piperazino, azetidino, morpholino, or thiomorpholino ring;

Rₕ is H, —(C₁-C₆)alkyl, —(C₂-C₆)alkenyl, —(C₂-C₆)alkynyl, —(C₃-C₆)cycloalkyl, heterocycle, heteroaryl or aryl;

each Rᵢ is independently selected from halogen, aryl, heteroaryl, heterocycle, R_{z}, OH, CN, —OR_{z}, —Oaryl, —OC(O)R_{z}, —OC(O)NR_{z1}R_{z2}, SH, —SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, —CHO, —C(O)R_{z}, —C(O)OH, —C(O)OR_{z}, —C(O)NR_{z1}R_{z2} and —C(O)C(O)R_{z}, wherein any aryl of Rᵢ may be optionally substituted with one or more (e.g. 1, 2, 3, 4 or 5) Rₘ groups;

Rⱼ and Rₖ are each independently selected from H, —(C₁-C₆)alkyl, —(C₂-C₆)alkenyl, —(C₂-C₆)alkynyl, —(C₃-C₆)cycloalkyl, aryl, aryl(C₁-C₆)alkyl-, heterocycle and heteroaryl; or Rⱼ and Rₖ together with the nitrogen to which they are attached form a pyrrolidino, piperidino, piperazino, azetidino, morpholino, or thiomorpholino ring;

each Rₘ is independently halogen, aryl, R_{z}, OH, CN, OR_{z}, —Oaryl, —Oheteroaryl, —OC(O)R_{z}, —OC(O)NR_{z1}R_{z2}, SH, SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, CHO, —C(O)R_{z}, —C(O)OH, —C(O)OR_{z}, —C(O)NR_{z1}R_{z2}, —C(O)C(O)R_{z}, heterocycle or heteroaryl;

Rₙ and Rₒ are each independently selected from H, —(C₁-C₆)alkyl, —(C₂-C₆)alkenyl, —(C₂-C₆)alkynyl, —(C₃-C₆)cycloalkyl, aryl, aryl(C₁-C₆)alkyl-, heterocycle and heteroaryl; or Rₙ and Rₒ together with the nitrogen to which they are attached form a pyrrolidino, piperidino, piperazino, azetidino, morpholino, or thiomorpholino ring;

each Rₚ is independently selected from halogen, aryl, heteroaryl, heterocycle, R_{z}, OH, CN, —OR_{z}, —Oaryl, —OC(O)R_{z}, —OC(O)NR_{z1}R_{z2}, oxo, SH, SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, —NOR_{z}, —CHO, —C(O)R_{z}, —C(O)OH, —C(O)OR_{z}, —C(O)NR_{z1}R_{z2} and —C(O)C(O)R_{z}, wherein any aryl of Rₚ may be optionally substituted with one or more (e.g. 1, 2, 3, 4 or 5) R_{y} groups;

R_{q} is —(C₁-C₆)alkyl, —(C₂-C₆)alkenyl, —(C₂-C₆)alkynyl, —(C₃-C₆)cycloalkyl, aryl, aryl(C₁-C₆)alkyl-, heterocycle and heteroaryl;

Rᵣ and Rₛ are each independently selected from H, —(C₁-C₆)alkyl, —(C₂-C₆)alkenyl, —(C₂-C₆)alkynyl, —(C₃-C₆)cycloalkyl, aryl, aryl(C₁-C₆)alkyl-, heterocycle and heteroaryl; or Rᵣ and Rₛ together with the nitrogen to which they are attached form a pyrrolidino, piperidino, piperazino, azetidino, morpholino, or thiomorpholino ring;

Rₜ is H, —(C₁-C₆)alkyl, —(C₂-C₆)alkenyl, —(C₂-C₆)alkynyl, —(C₃-C₆)cycloalkyl, aryl, aryl(C₁-C₆)alkyl-, heterocycle and heteroaryl;

each R_{w} is independently (C₁-C₆)alkyl, —O(C₁-C₆)alkyl, —C(O)NRⱼRₖ, halogen, CF₃, CN or NHC(O)Rₕ;

each R_{y} is independently halogen, R_{z}, OH, CN, OR_{E}, —Oaryl, —Oheteroaryl, —OC(O)R_{z}, —OC(O)NR_{z1}R_{z2}, SH, SR₁, —Saryl, —Sheteroaryl, —S(O)R_{z}, —S(O)aryl, —S(O)heteroaryl, —S(O)₂OH, —S(O)₂OR_{z}, —S(O)₂R_{z}, —OS(O)₂R_{z}, —S(O)₂Oaryl, —S(O)₂aryl, —OS(O)₂aryl, —S(O)₂heteroaryl, —OS(O)₂heteroaryl, —S(O)₂NR_{z1}R_{z2}, —S(O) NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, ═NOH, ═NOR_{z}, —C(NH₂)(═NCN), CHO, —C(O)R_{z}, —C(O)OH, —C(O)Oaryl, —C(O)OR_{z}, —C(O)NR_{z1}R_{z2}, —C(O)aryl, —OC(O)aryl, —C(O)heteroaryl, —OC(O)heteroaryl, —C(O)C(O)R_{z}, ═CR_{z7}R_{z8}, aryl, heterocycle or heteroaryl, wherein any aryl or heteroaryl of R_{y} is optionally substituted with one or more (e.g. 1, 2, 3, 4 or 5) halogen, R_{z}, (C₂-C₆)alkynyl, —OR_{z}, CN, NR_{z1}R_{z2}, —NO₂, —CHO, —Oaryl, —C(O)OR_{z}, —C(O)OH, —NHCOR_{z}, —NHS(O)₂R_{z}, —NHS(O)₂aryl, —NHS(O)₂heteroaryl, —C(O)NR_{z1}R_{z2}, —NHCONR_{z1}R_{z2}, —NHC(O)OR_{z}, —NHCOaryl, —NHCOheteroaryl, —NHC(O)OR_{z}, —(C₂-C₆)alkynyl, —S(O)₂NR_{z1}R_{z2}, —S(O)₂R_{z}, —S(O)₂aryl, —S(O)₂heteroaryl, —S(O)₂(C₃-C₆)cycloalkyl, —S(O)R_{z}, —S(O)aryl, —S(O)heteroaryl, —S(O)(C₃-C₆)cycloalkyl, —SR_{z}, —S(C₁-C₆)alkyl aryl, heteroaryl or heterocycle, wherein aryl or heteroaryl is optionally substituted with one or more (e.g. 1, 2, 3, 4 or 5) halogen, CF₃, CN or (C₁-C₃)alkyl and wherein any heterocycle of R_{y} is optionally substituted with one or more (e.g. 1, 2, 3, 4 or 5) oxo, R_{z}, —S(O)₂R_{z}, —S(O)₂aryl, —S(O)₂heteroaryl, —C(O)R_{z}, —C(O)aryl, —C(O)heteroaryl or heteroaryl wherein aryl or heteroaryl is optionally substituted with one or more (e.g. 1, 2, 3, 4 or 5) halogen or (C₁-C₃)alkyl;

each R_{z} is independently —(C₁-C₆)alkyl or —(C₃-C₆)cycloalkyl wherein alkyl may be optionally substituted with one or more (e.g. 1, 2, 3, 4 or 5) R_{z4} groups, wherein cycloalkyl may be optionally substituted with one or more (e.g. 1, 2, 3, 4 or 5) groups selected from R_{z4}, —(C₁-C₆)alkyl and —(C₁-C₆)alkylOH;

R_{z1} and R_{z2} are each independently selected from H, —(C₁-C₆)alkyl, —(C₂-C₆)alkenyl, —(C₂-C₆)alkynyl, —(C₃-C₆)cycloalkyl, aryl, heterocycle and heteroaryl, wherein any alkyl, —(C₂-C₆)alkenyl or —(C₂-C₆)alkynyl of R_{z1} or R_{z2} may be optionally substituted with one or more (e.g. 1, 2, 3, 4 or 5) R_{z3} groups and wherein aryl or heteroaryl of R_{z1} or R_{z2} may be optionally substituted with one or more (e.g. 1, 2, 3, 4 or 5) —(C₁-C₆)alkyl or R_{z3} groups and wherein any heterocycle or cycloalkyl of R_{z1} or R_{z2} may be optionally substituted with or more (e.g. 1, 2, 3, 4 or 5) —(C₁-C₆)alkyl, oxo or R_{z3} groups; or R_{z1} and R_{z2} together with the nitrogen to which they are attached form a cyclic amino optionally substituted with one or more (e.g. 1, 2, 3, 4 or 5) —(C₁-C₆)alkyl, oxo or R_{z3} groups;

each R_{z3} is independently selected from halogen, CN, CF₃, NR_{z5}R_{z6}, OH, —O(C₁-C₆)alkyl, —C(O)NR_{z5}R_{z6}, —C(O)(C₁-C₆)alkyl, aryl, heterocycle and heteroaryl, wherein any heterocycle of R_{z3} may be substituted with one or more (e.g. 1, 2, 3, 4 or 5) —(C₁-C₆)alkyl;

each R_{z4} is independently selected from halogen, CN, OH, —NR_{z5}R_{z6}, —SCN, —O(C₁-C₆)alkyl, —Sheteroaryl, —S(O)aryl, —S(O)₂aryl, —Oaryl, —C(O)NR_{z5}R_{z6}, (C₃-C₆)cycloalkyl, —CH₂NHCOaryl, —CH₂OCH₂aryl, biphenyl, aryl, heterocycle and heteroaryl, wherein any aryl, heteroaryl or heterocycle of R_{z4} may be optionally substituted with one or more (e.g. 1, 2, 3, 4 or 5) halogen, CN, —(C₁-C₆)alkyl, —NH₂, —NHheteroaryl, —NHS(O)₂(C₁-C₆)alkyl or —O(C₁-C₆)alkyl;

R_{z5} and R_{z6} are each independently selected from H or —(C₁-C₆)alkyl wherein alkyl is optionally substituted with NH₂; and

R_{z7} and R_{z8} together with the atom to which they are attached form a —(C₃-C₆)cycloalkyl; or a salt thereof.

A specific compound of the invention is:

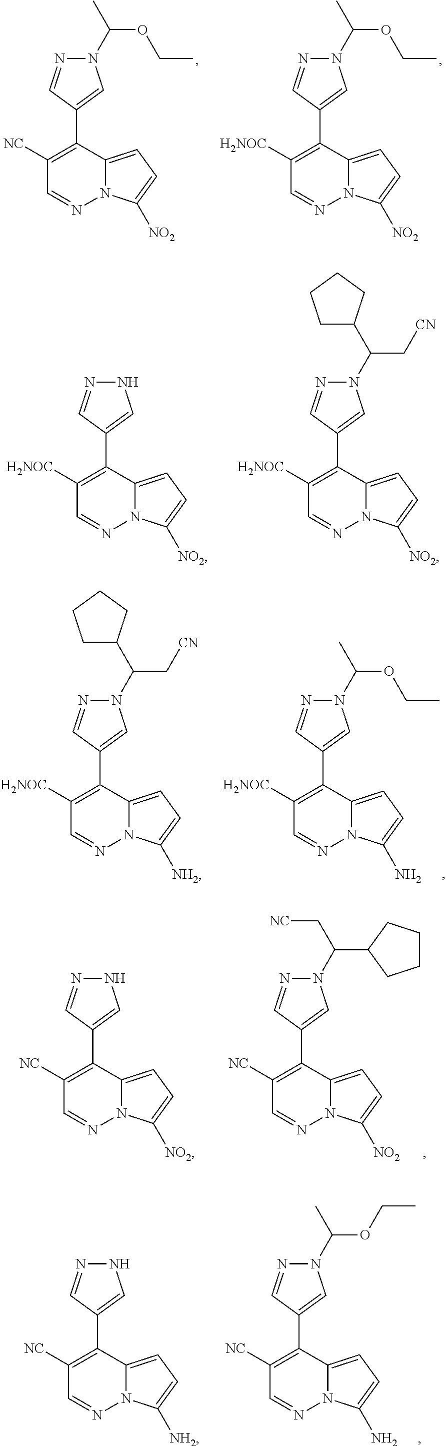

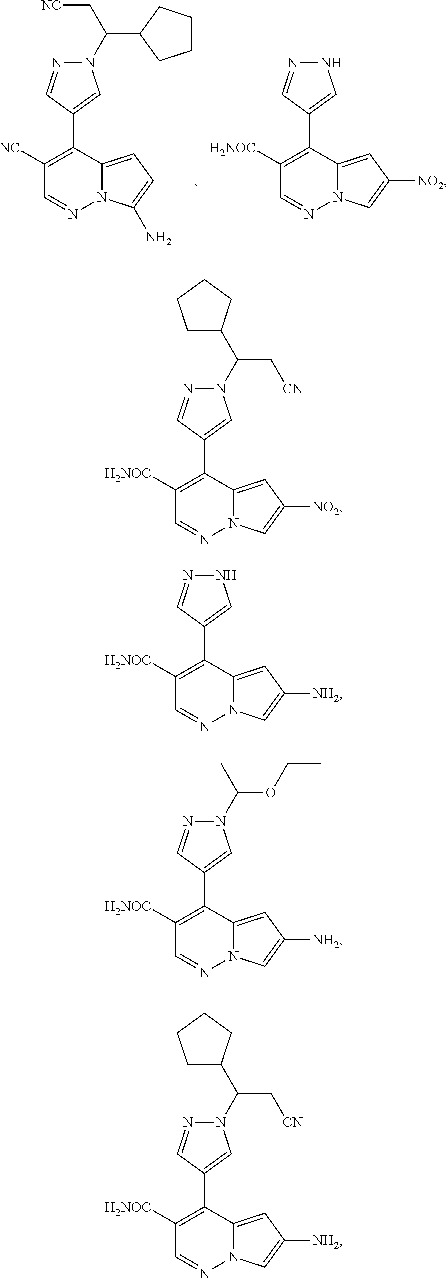

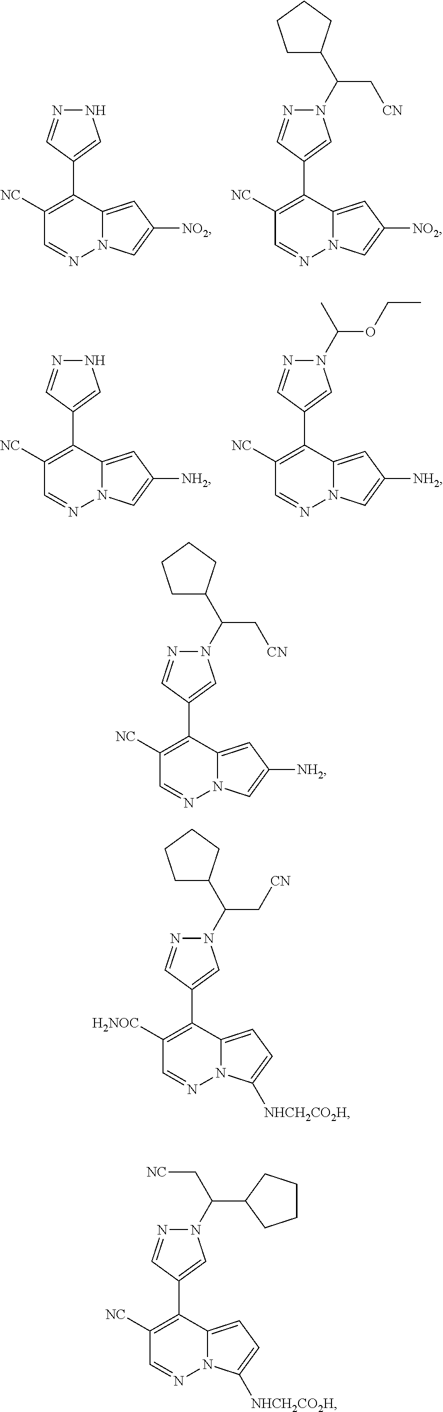

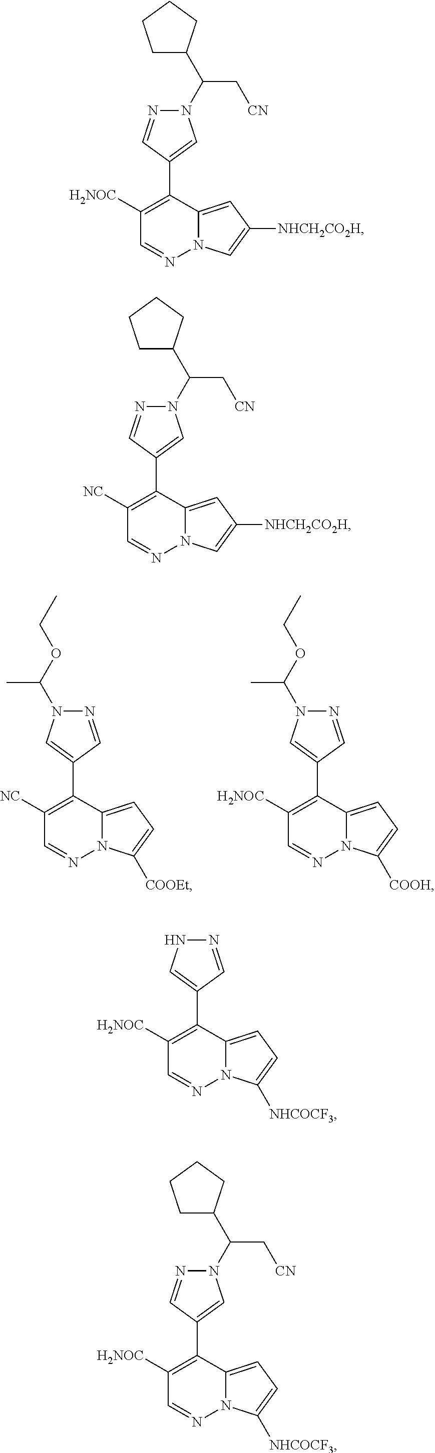

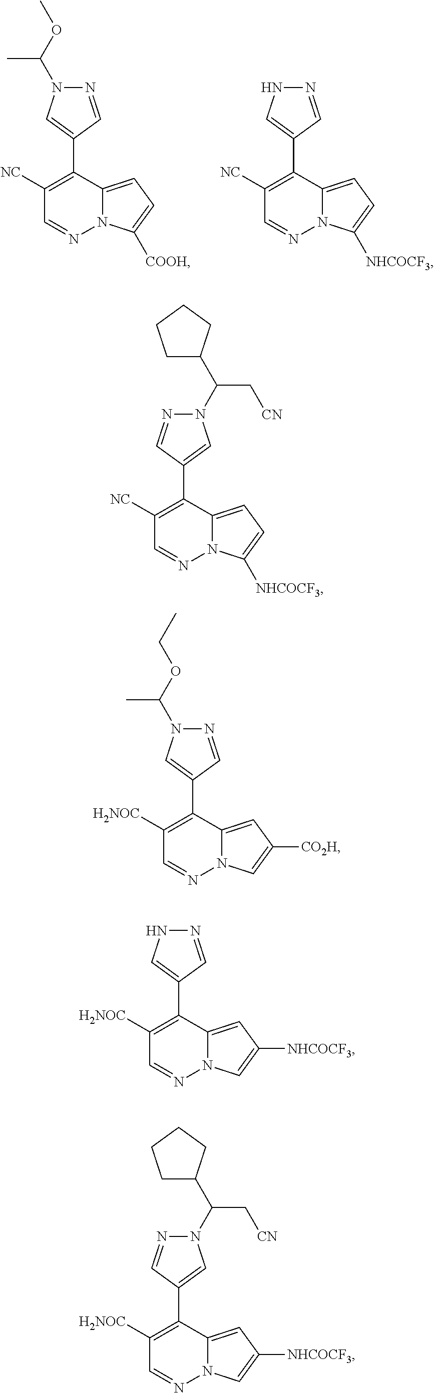

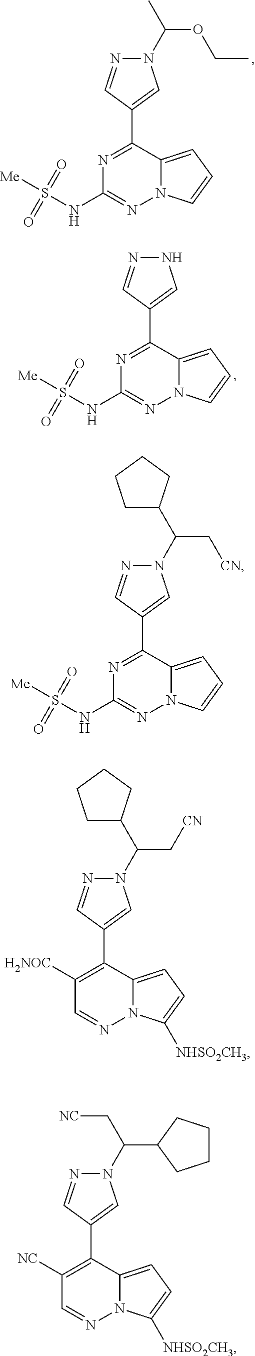

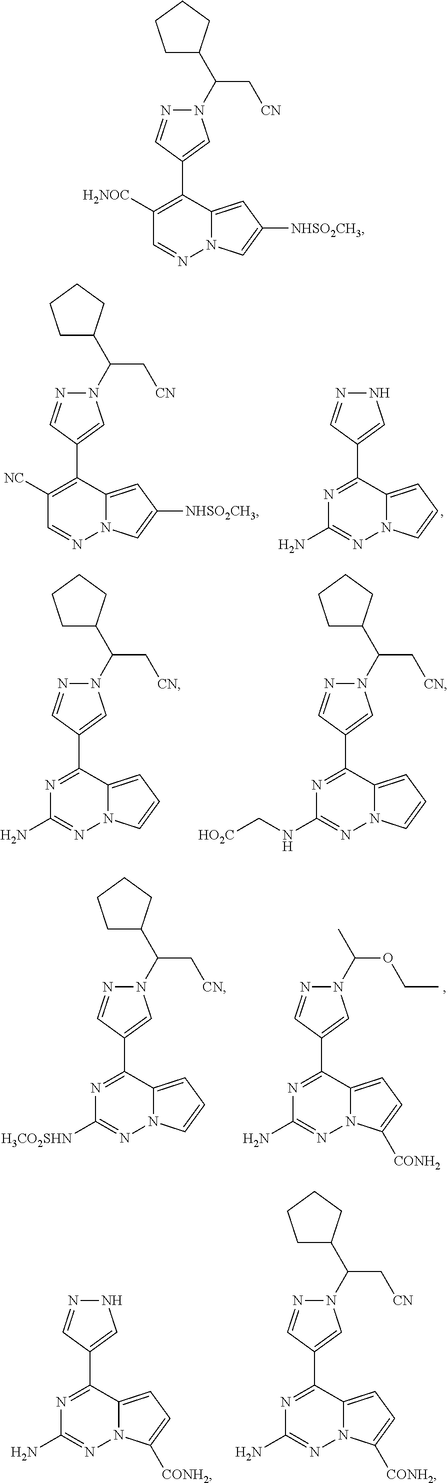

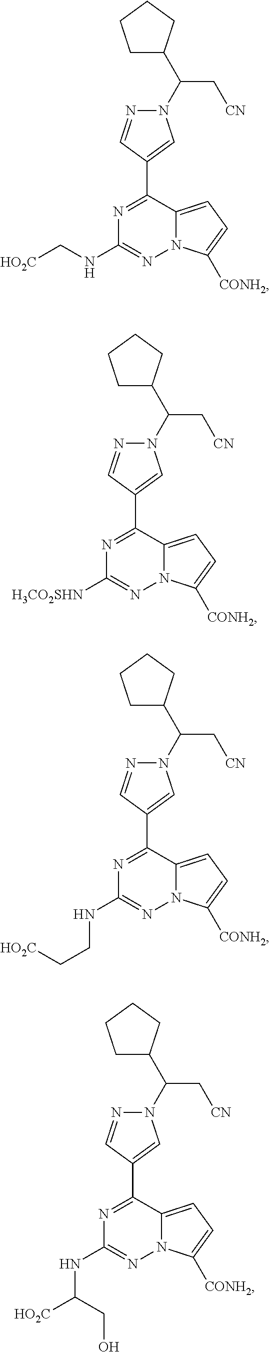

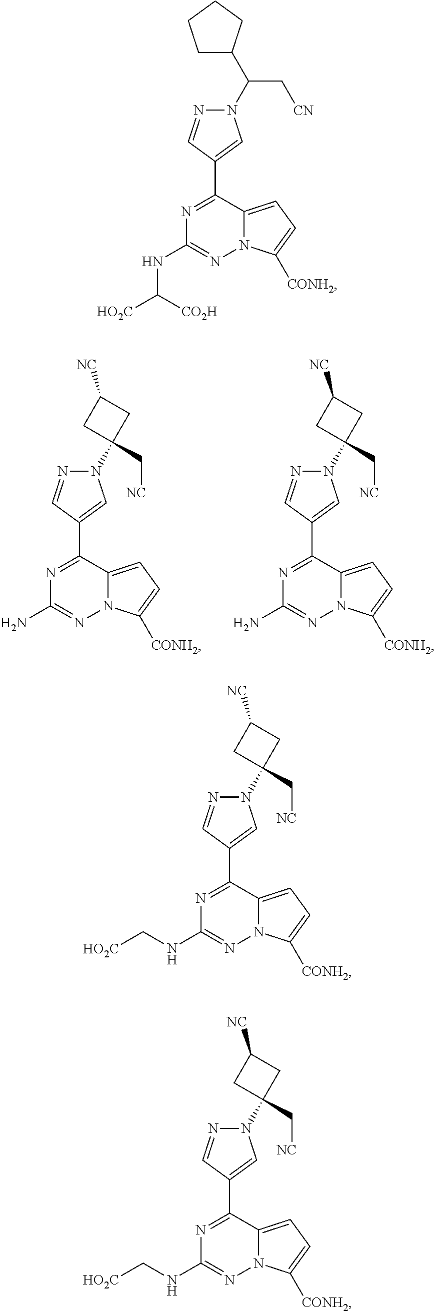

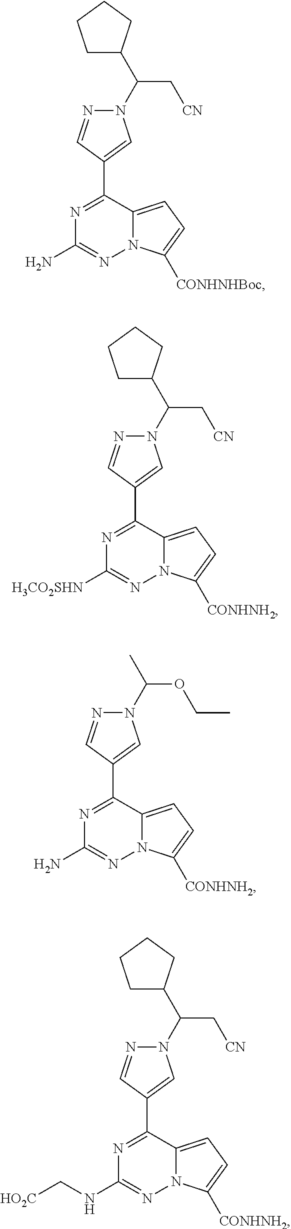

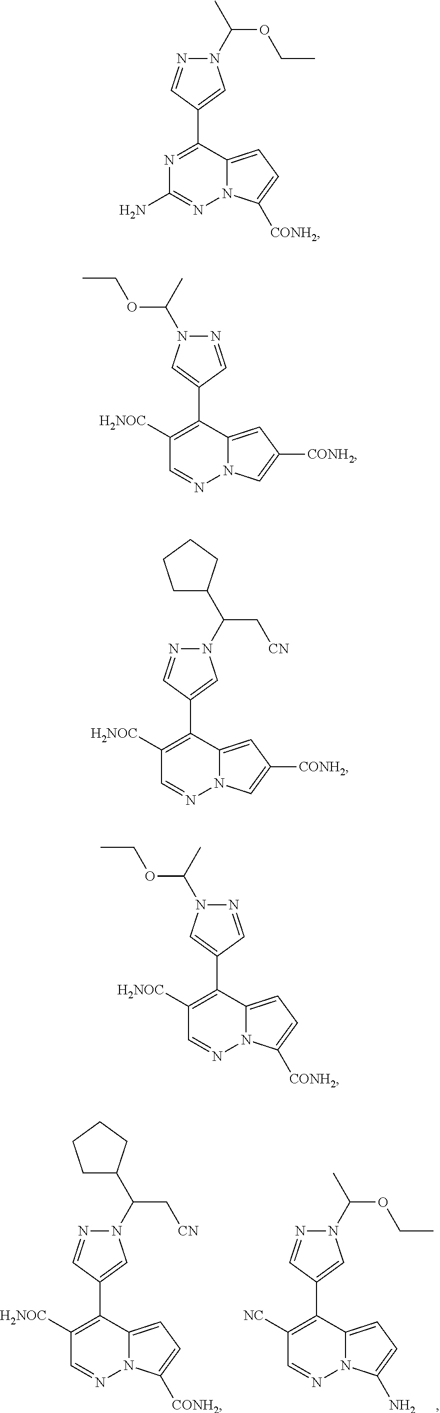 [SMILES: CCOC(C)n1cc(-c2nc(N)nn3c(C(N)=O)ccc23)cn1 ; CCOC(C)n1cc(-c2c(C(N)=O)cnn3cc(C(N)=O)cc23)cn1 ; N#CCC(C1CCCC1)n1cc(-c2c(C(N)=O)cnn3cc(C(N)=O)cc23)cn1 ; CCOC(C)n1cc(-c2c(C(N)=O)cnn3c(C(N)=O)ccc23)cn1 ; N#CCC(C1CCCC1)n1cc(-c2c(C(N)=O)cnn3c(C(N)=O)ccc23)cn1 ; CCOC(C)n1cc(-c2c(C#N)cnn3c(N)ccc23)cn1]

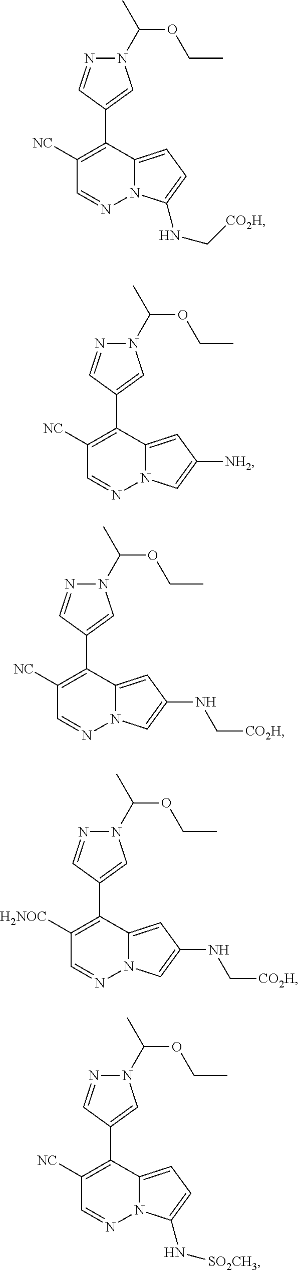

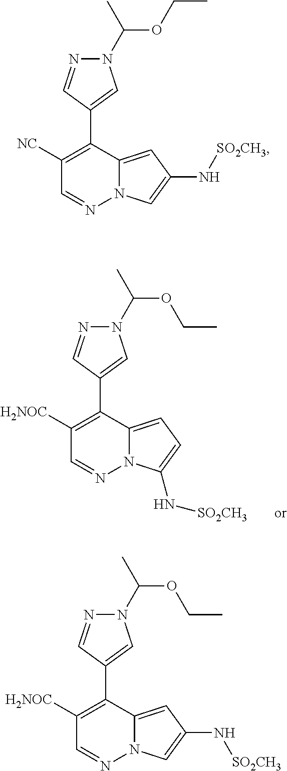 [SMILES: CCOC(C)n1cc(-c2c(C#N)cnn3cc(NS(C)(=O)=O)cc23)cn1 ; CCOC(C)n1cc(-c2c(C(N)=O)cnn3c(NS(C)(=O)=O)ccc23)cn1 ; CCOC(C)n1cc(-c2c(C(N)=O)cnn3cc(NS(C)(=O)=O)cc23)cn1]

or a salt thereof.

Another specific compound of the invention is:

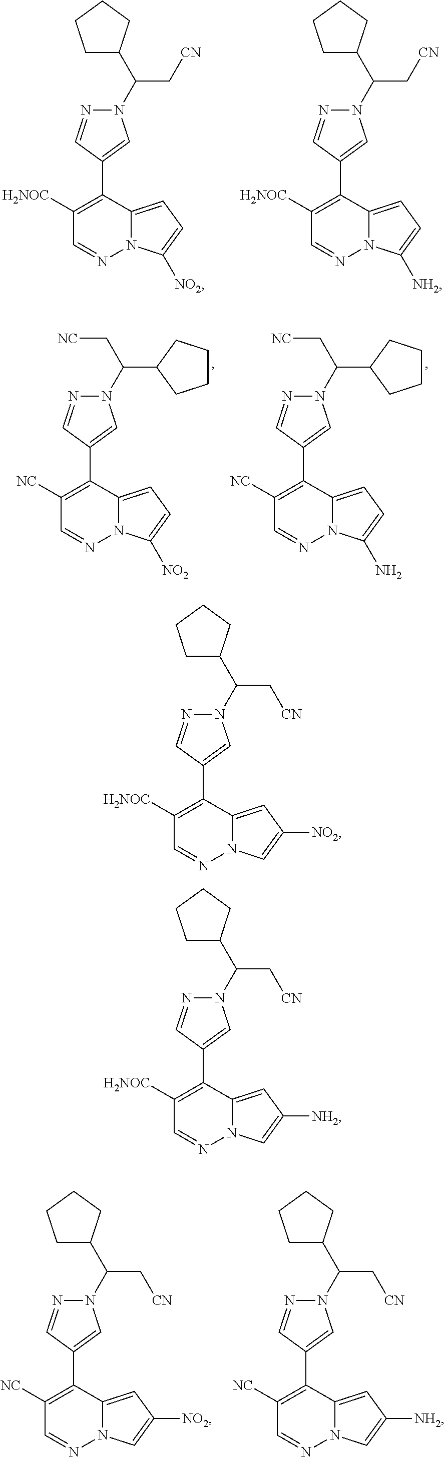

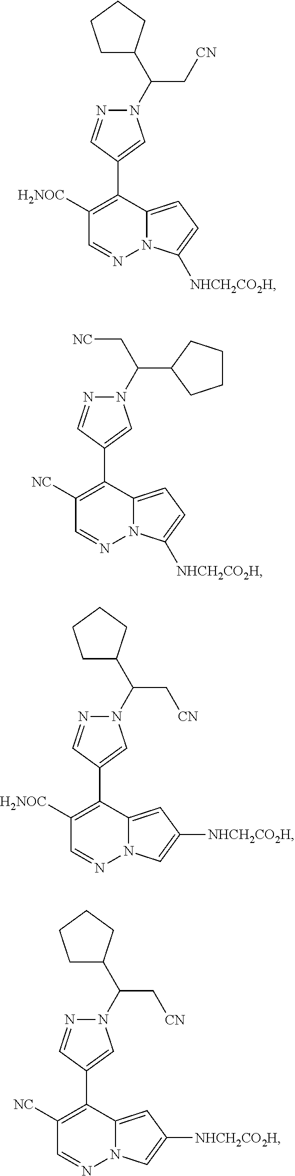

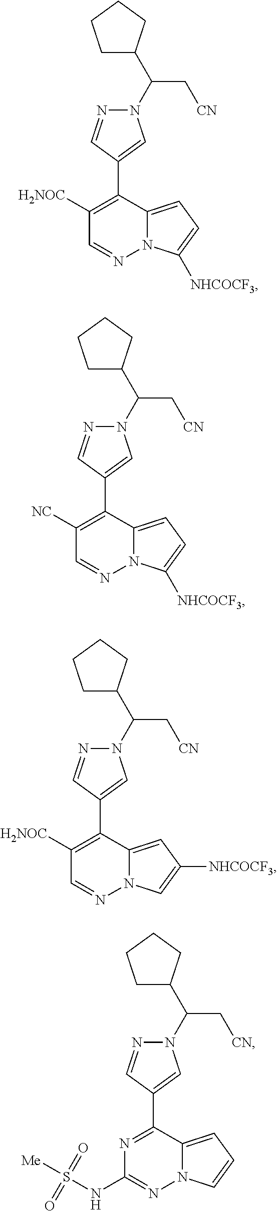

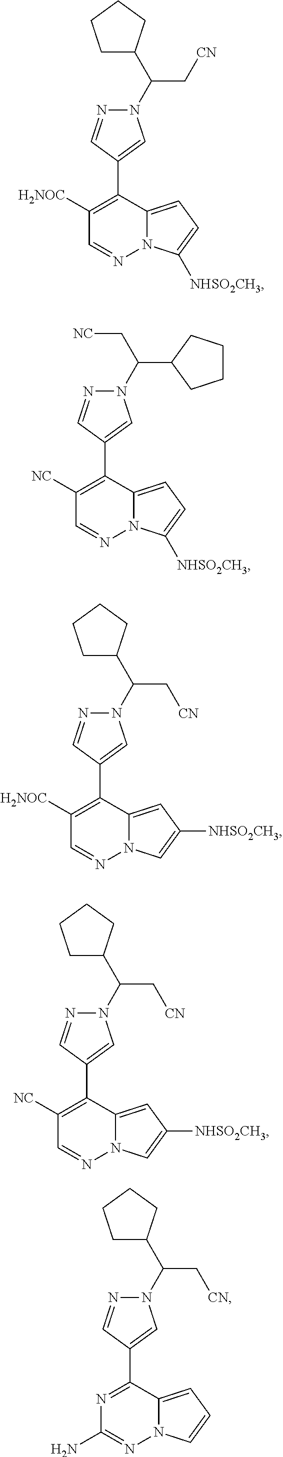

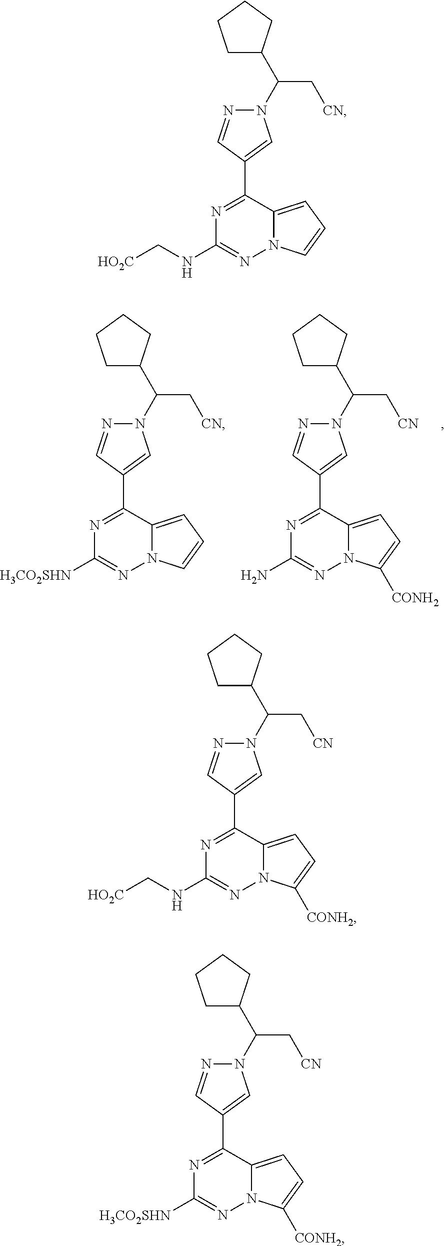

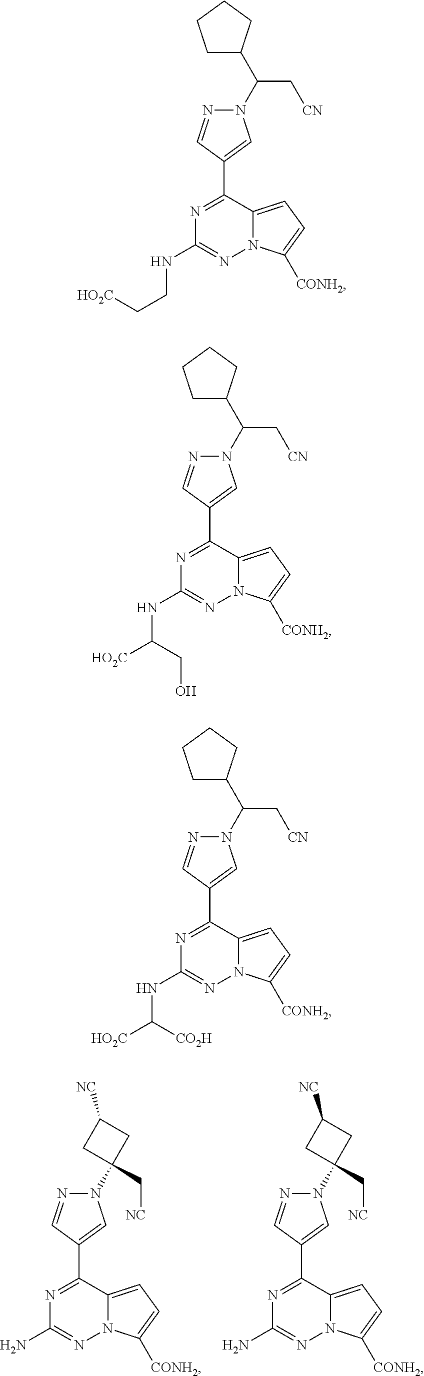 [SMILES: N#CCC(C1CCCC1)n1cc(-c2nc(NCCC(=O)O)nn3c(C(N)=O)ccc23)cn1 ; N#CCC(C1CCCC1)n1cc(-c2nc(NC(CO)C(=O)O)nn3c(C(N)=O)ccc23)cn1 ; N#CCC(C1CCCC1)n1cc(-c2nc(NC(C(=O)O)C(=O)O)nn3c(C(N)=O)ccc23)cn1 ; N#CC[C@]1(n2cc(-c3nc(N)nn4c(C(N)=O)ccc34)cn2)C[C@@H](C#N)C1 ; N#CC[C@]1(n2cc(-c3nc(N)nn4c(C(N)=O)ccc34)cn2)C[C@H](C#N)C1]

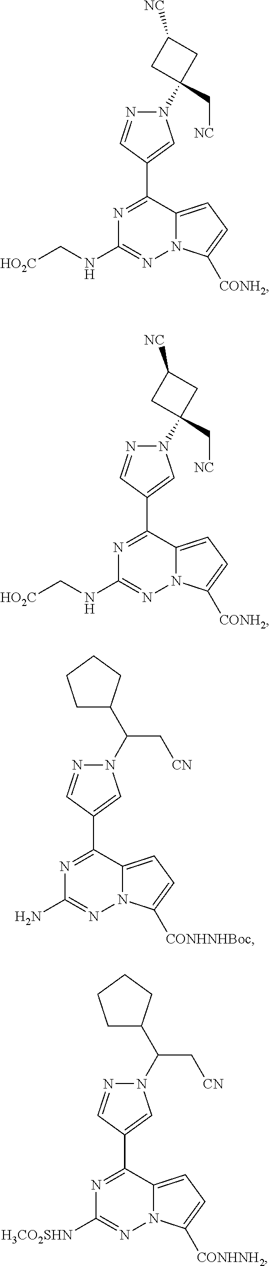

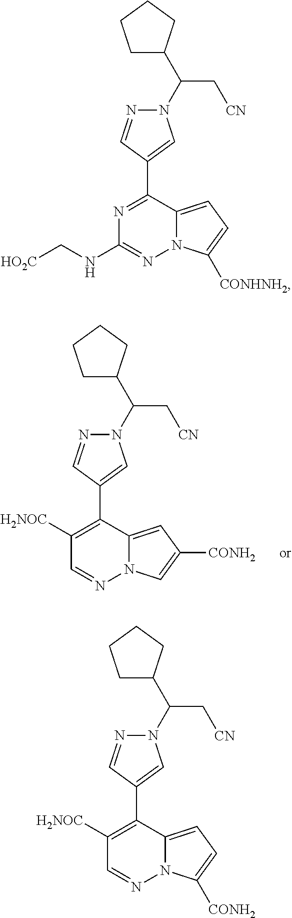

or a salt thereof.

Another specific compound of the invention is:

3-cyclopentyl-3-(4-(pyrrolo[1,2-f][1,2,4]triazin-4-yl)-1H-pyrazol-1-yl)propanenitrile;

4-(1-(1-ethoxyethyl)-1H-pyrazol-4-yl)pyrrolo[1,2-f][1,2,4]triazine;

4-(1H-pyrazol-4-yl)pyrrolo[1,2-f][1,2,4]triazine;

4-(1H-pyrazol-4-yl)pyrrolo[1,2-b]pyridazine-3-carboxamide;

4-(1-(1-ethoxyethyl)-1H-pyrazol-4-yl)pyrrolo[1,2-b]pyridazine-3-carbonitrile;

4-(1-(1-ethoxyethyl)-1H-pyrazol-4-yl)pyrrolo[1,2-b]pyridazine-3-carboxamide ;

4-(1-(2-cyano-1-cyclopentylethyl)-1H-pyrazol-4-yl)pyrrolo[1,2-b]pyridazine-3-carboxamide;

4-(1-(1-ethoxyethyl)-1H-pyrazol-4-yl)-6-nitropyrrolo[1,2-b]pyridazine-3-carboxamide;

4-(1H-pyrazol-4-yl)pyrrolo[1,2-b]pyridazine-3-carbonitrile;

4-(1-(2-cyano-1-cyclopentylethyl)-1H-pyrazol-4-yl)pyrrolo[1,2-b]pyridazine-3-carbonitrile;

methyl (4-(1-(1-ethoxyethyl)-1H-pyrazol-4-yl)pyrrolo[2,1-f][1,2,4]triazin-2-yl)carbamate;

4-(1-(1-ethoxyethyl)-1H-pyrazol-4-yl)pyrrolo[2,1-f][1,2,4]triazin-2-amine; or

4-(1H-pyrazol-4-yl)pyrrolo[2,1-f][1,2,4]triazin-2-amine

or a salt thereof.

### Tautomers:

A wide variety of functional groups and other structures exhibit tautomerism and all tautomers of compounds of formula I are within the scope of the present invention. For example, pyrazoles may exhibit the isomeric forms referred as tautomers.

Tautomers are isomeric forms of a compound that are in equilibrium with each other. The concentrations of the isomeric forms will depend on the environment in which the compound is found and may be different depending on if the compound is a solid or is in an organic or aqueous solution.

Processes which were used or can be used to prepare compounds of formula I are provided as further embodiments of the invention and are illustrated in Schemes 1, 2, 3, 4, 7, 8, 9, 10, 13, 14 and 15-33. Additional processes which can be used to prepare intermediates useful for preparing compounds of formula I are provided in Schemes 5, 6, 11 and 12.

### General Methods of Preparation of Invention Compounds:

Heterocycles can be prepared from known methods as reported in the literature (a. Ring system handbook, published by American Chemical Society edition 1993 and subsequent supplements. b. The Chemistry of Heterocyclic Compounds; Weissberger, A., Ed.; Wiley: N.Y., 1962. c. Nesynov, E. P.; Grekov, A. P. The chemistry of 1,3,4-oxadiazole derivatives. Russ. Chem. Rev. 1964, 33, 508-515. d. Advances in Heterocyclic Chemistry; Katritzky, A. R., Boulton, A. J., Eds.; Academic Press: New York, 1966. e. In Comprehensive Heterocyclic Chemistry; Potts, K. T., Ed.; Pergamon Press: Oxford, 1984. f. Eloy, F. A review of the chemistry of 1,2,4-oxadiazoles. Fortschr. Chem. Forsch. 1965, 4, pp 807-876. g. Adv. Heterocycl. Chem. 1976. h. Comprehensive Heterocyclic Chemistry; Potts, K. T., Ed.; Pergamon Press: Oxford, 1984. i. Chem. Rev. 1961 61, 87-127. j. 1,2,4-Triazoles; John Wiley & Sons: New York,1981; Vol 37). Some of the functional groups during the synthesis may need to be protected and subsequently deprotected. Examples of suitable protecting groups can be found in “Protective groups in organic synthesis” fourth edition edited by Greene and Wuts.

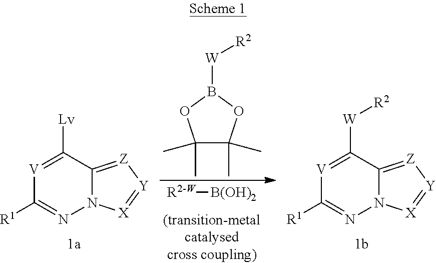

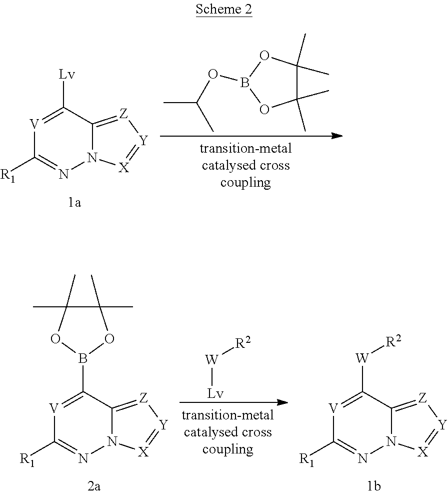

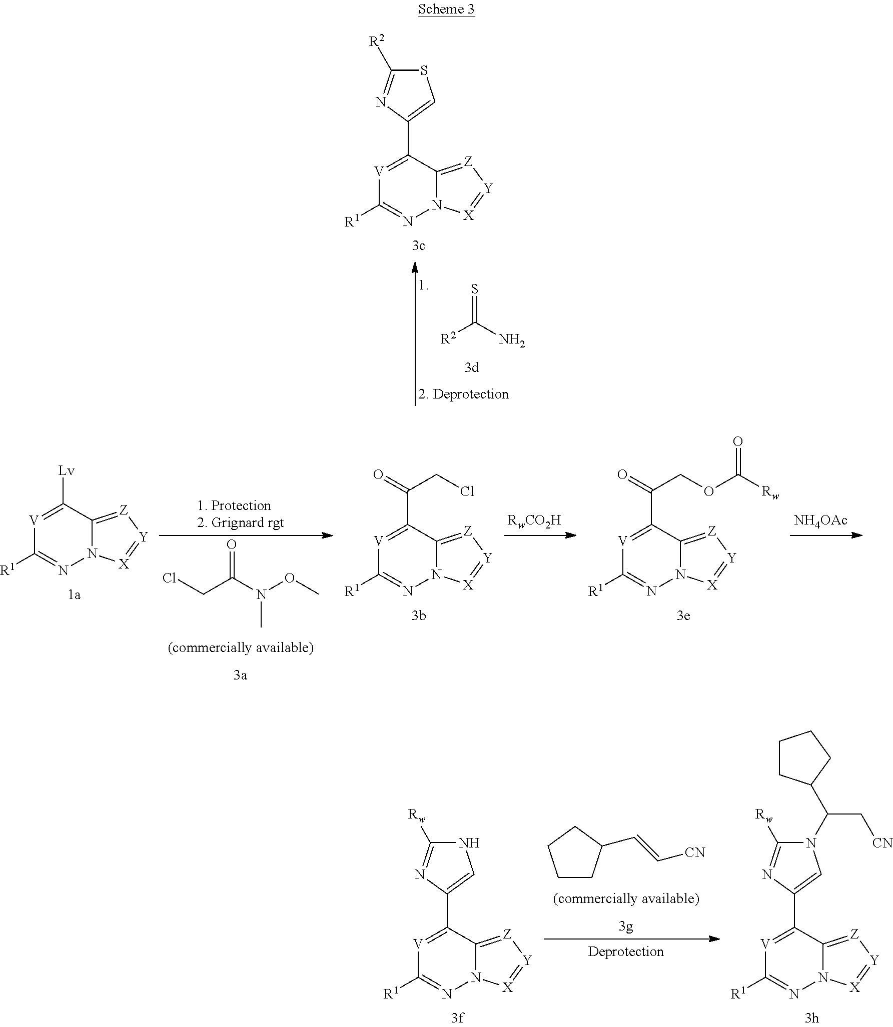

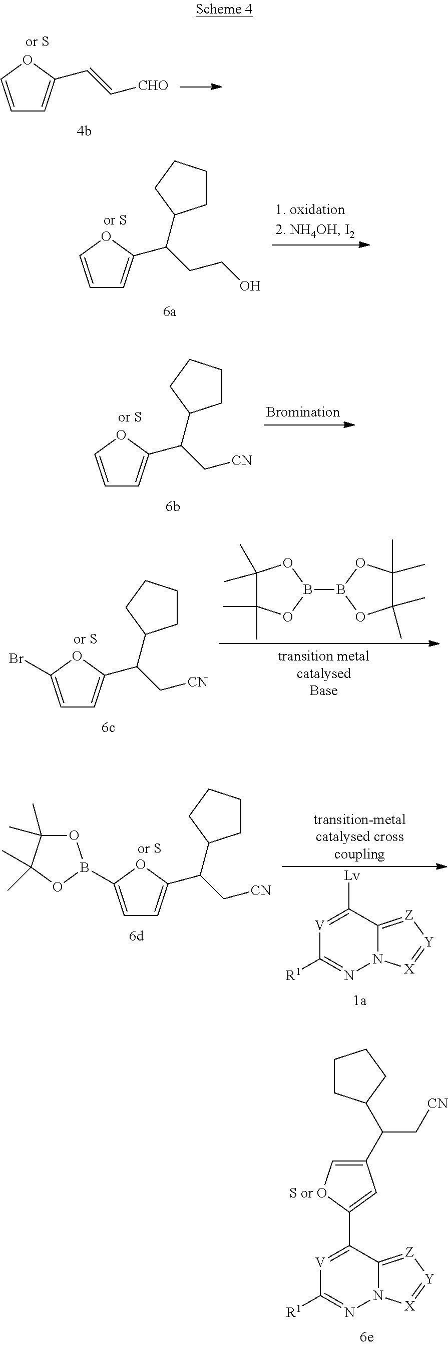

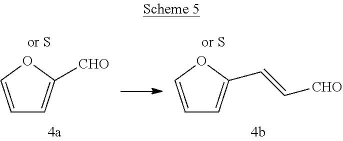 [SMILES: O=Cc1ccco1 ; O=C/C=C/c1ccco1]

The intermediate 3-(furan-2-yl)acrylaldehyde (4b) can be prepared as depicted in Scheme 5 from furan-2-carbaldehyde 4(a) according to the following procedures reported in the literature a) Valenta, Petr; Drucker, Natalie A.; Bode, Jeffrey W.; Walsh, Patrick J; Organic Letters 2009, 11(10), 2117-2119. b) McComsey, David F.; Maryanoff, Bruce E. Encyclopedia of Reagents for Organic Synthesis (2001) c) Mahata, Pranab Kumar; Barun, Okram; IIa, H.; Junjappa, H. Synlett 2000, 9, 1345-1347. d) Shapiro, Yu. M. Krasnodar. Khimiya Geterotsiklicheskikh Soedinenii 1993, 1, 25-8. e) Bellassoued, Moncef; Majidi, Assieh. Journal of Organic Chemistry 1993, 58(9), 2517-22. f) Duhamel, L.; Gralak, J.; Ngono, B. Journal of Organometallic Chemistry 1989, 363(1-2), C4-C6. g) Di Nunno, L.; Scilimati, A. Tetrahedron 1988, 44(12), 3639-44. h) Duhamel, Lucette; Ple, Gerard; Organic Preparations and Procedures International 1986, 18(4), 219-26. i) Bestmann, Hans Juergen; Roth, Kurt; Ettlinger, Manfred. Chemische Berichte 1982, 115(1), 161-71. j) Bestmann, Hans Juergen; Roth, Kurt; Ettlinger, Manfred. Angewandte Chemie 1979, 91(9), 748.

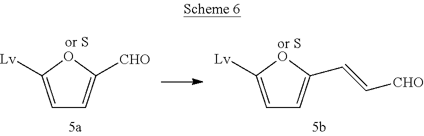

The intermediate 3-(furan-2-yl)acrylaldehyde (5b) can be prepared as depicted in Scheme 6 from the appropriately substituted furan-2-carbaldehyde 5(a) according to the following procedure reported in the literature Mocelo, R.; Pustovarov, V. Esc. Quim., Univ. La Habana, Havana, Cuba. Revista sobre los Derivados de la Cana de Azucar (1976), 10(2), 3-9.

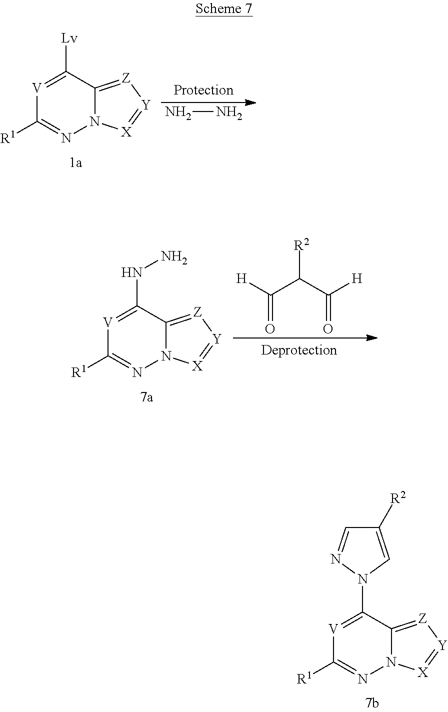

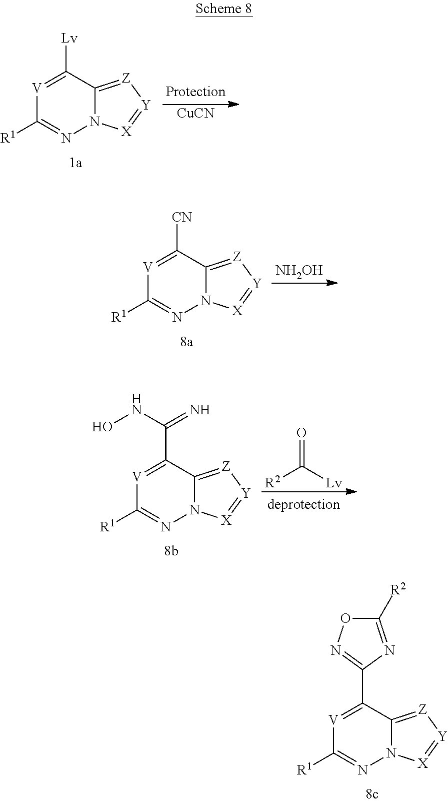

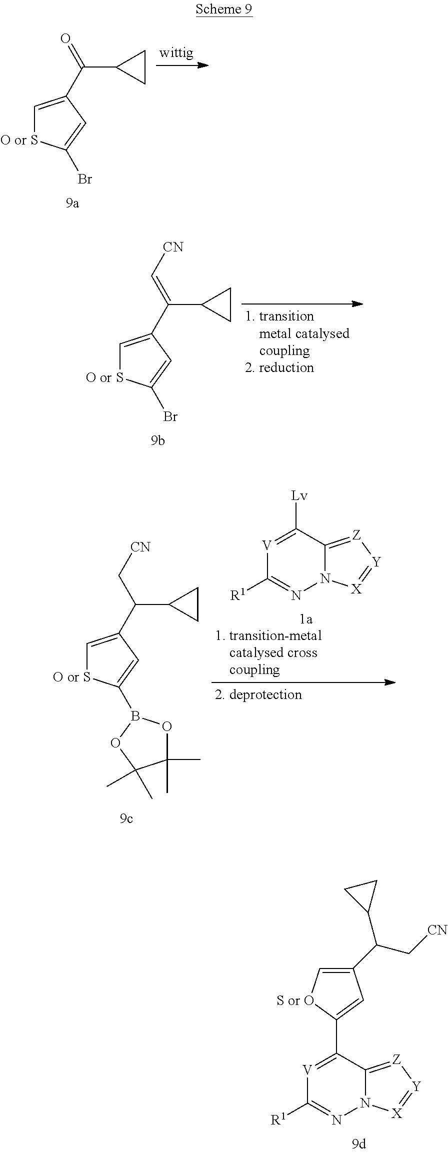

Compound 9a can be prepared following known procedures (WO 2001023383, JP07285931, JP06345772 and EP629626).

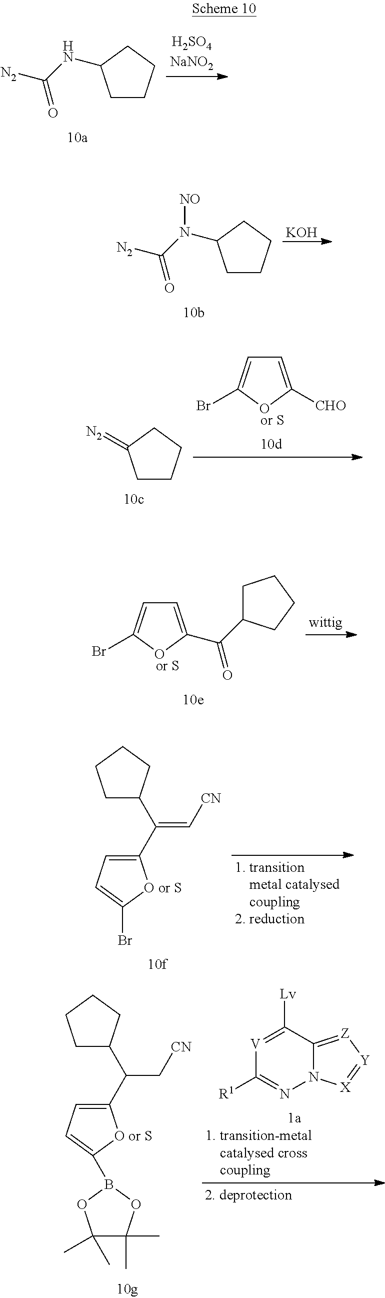

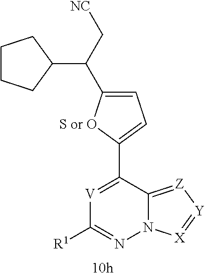

Alkyl groups like cyclopentyl can be incorporated into the compounds of the invention according to procedure in Scheme 10. Diazotization of commercially available 1-cyclopentylurea 10a to 1-cyclopentyl-1-nitrosourea 10b can be achieved by using conditions reported by Afshar, DaAghaei; Islami, Mohammad Reza. Journal of Chemical Research 2008, (9), 509-511. Diazocyclopentane 10c can be prepared from 1-cyclopentylurea 10b by using reaction conditions reported in Berthon-Gelloz, Guillaume; Marchant, Melanie; Straub, Bernd F.; Marko, Istvan E. Chemistry—A European Journal 2009, 15(12), 2923-2931. Reaction of diazocyclopentane 10c with 5-bromofuran-2-carbaldehyde or 5-bromothiophene-2-carbaldehyde 10d follows conditions reported by Sarma, C. R.; Krishna, R. R.; Shridhar, D. R.; Rao, C. Seshagiri; Taneja, V. Indian Journal of Chemistry, Section B: Organic Chemistry Including Medicinal Chemistry (1989), 28B(11), 993-5, gives (5-bromofuran-2-yl)(cyclopentyl)methanone or (5-bromothiophen-2-yl)(cyclopentyl)methanone 10e. The final compound can be derived from 10e by following a similar protocol as reported in Scheme 9.

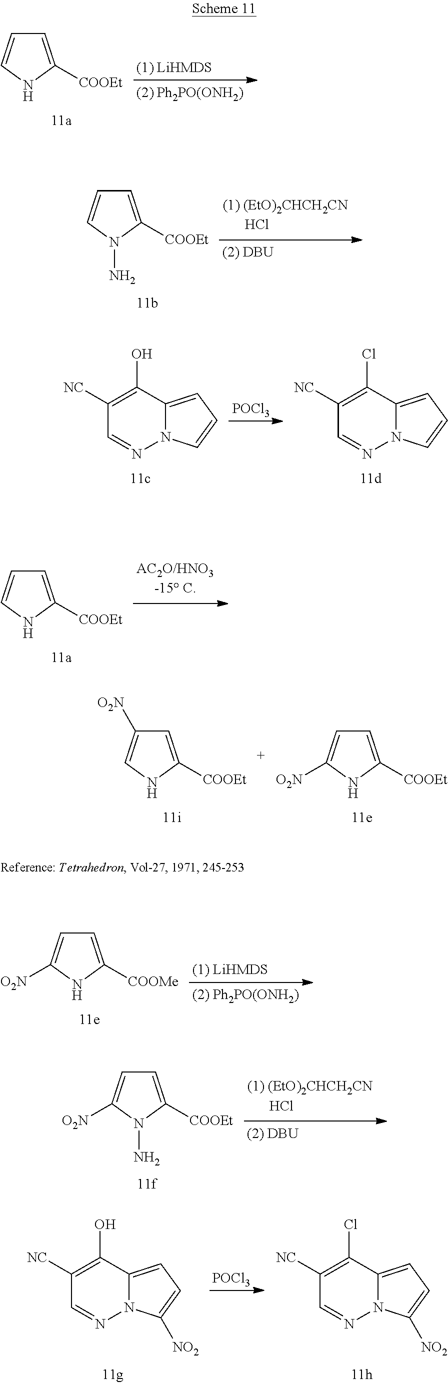

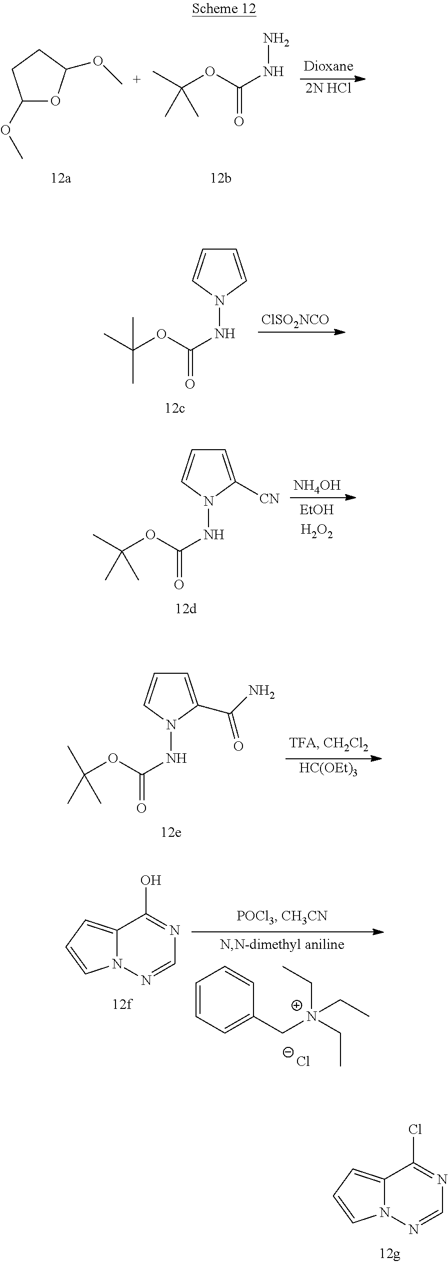

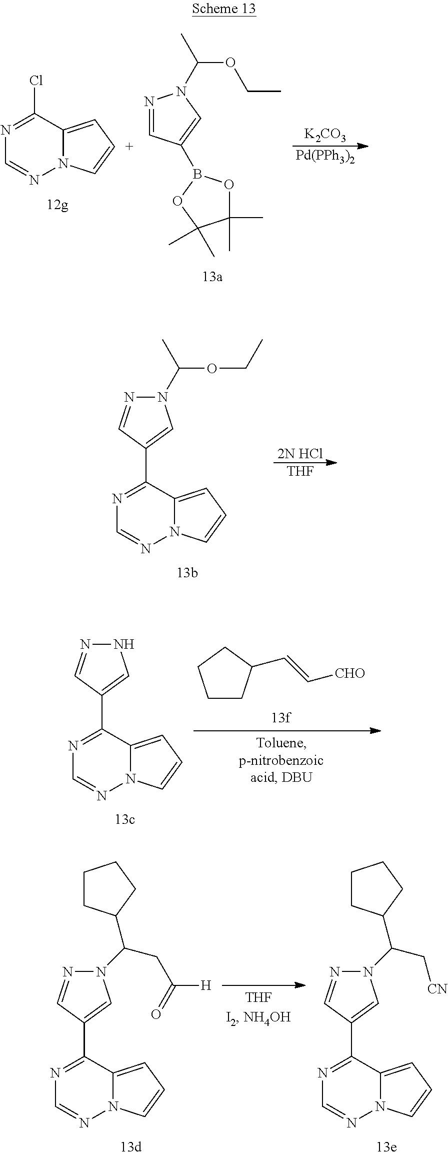

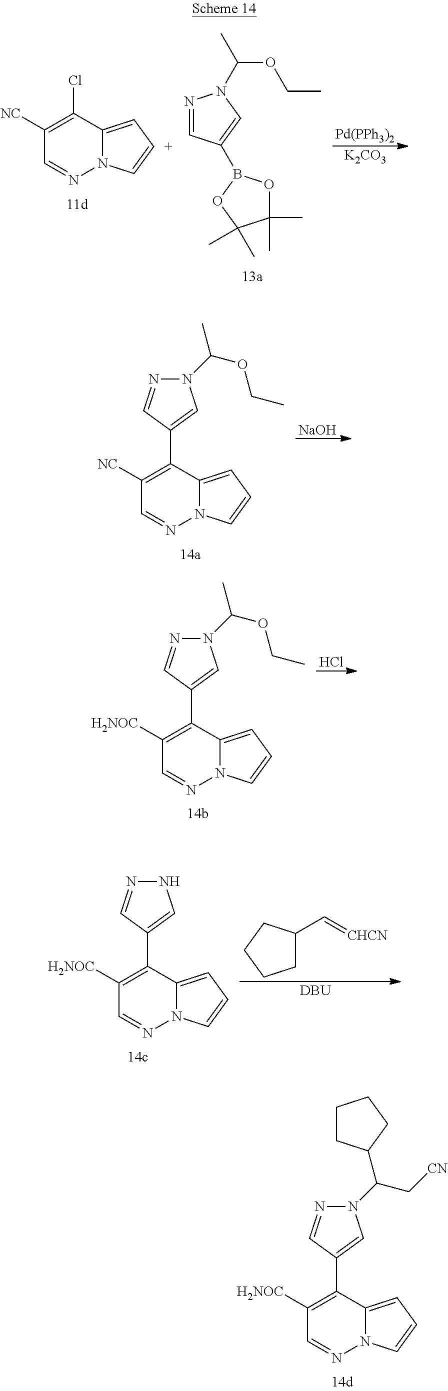

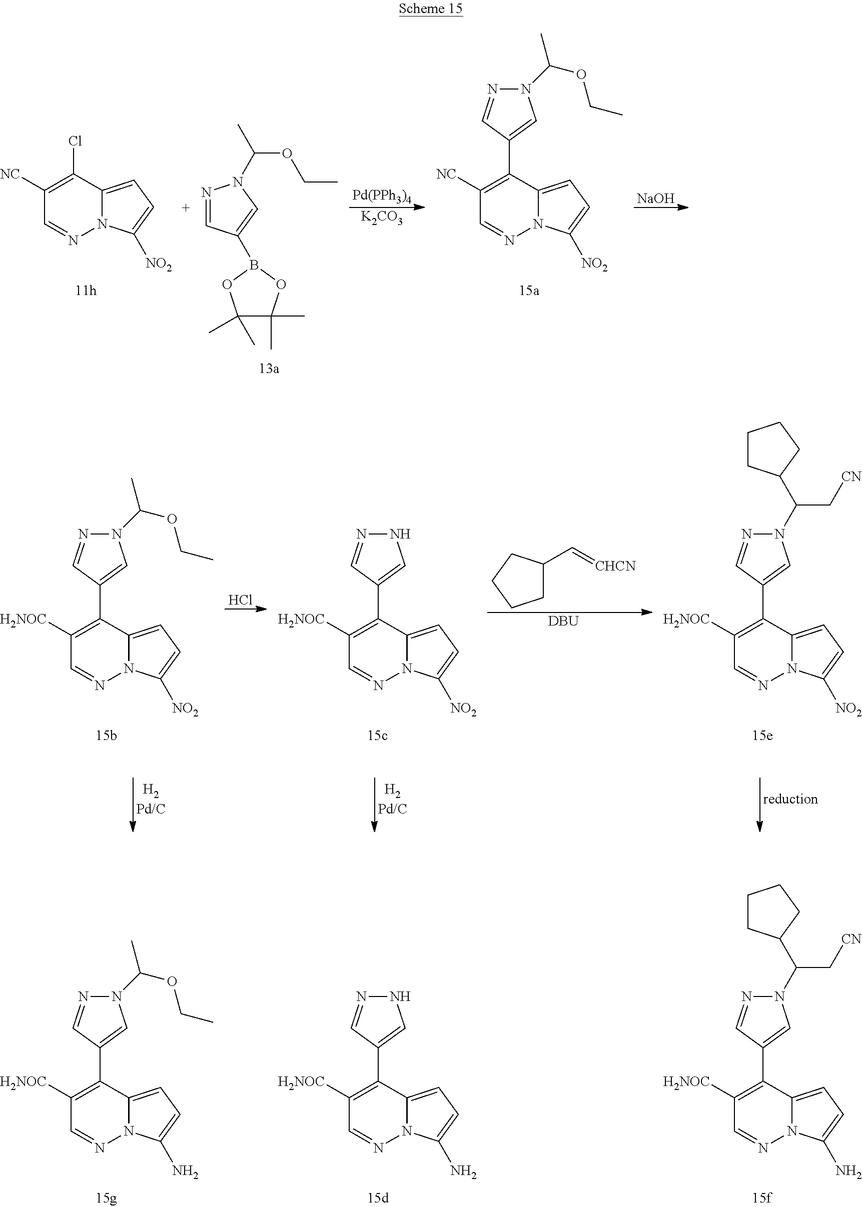

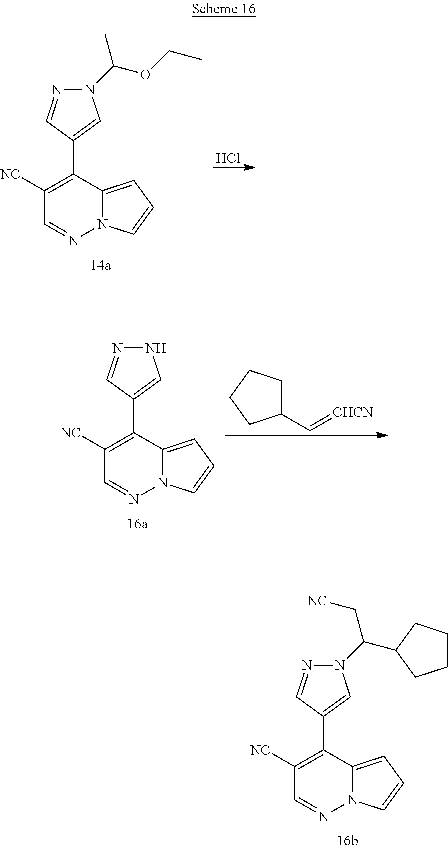

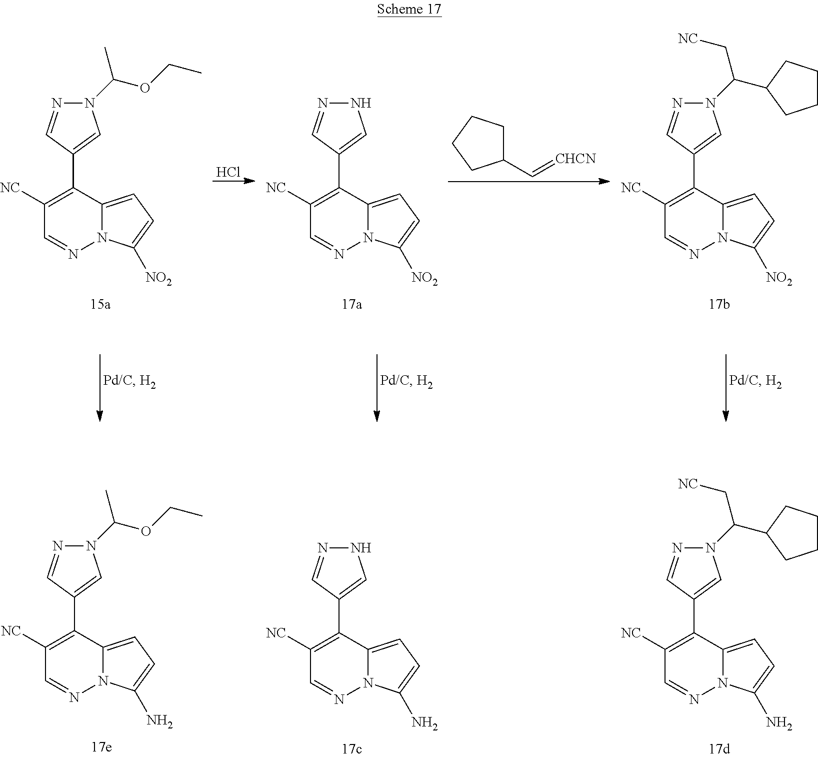

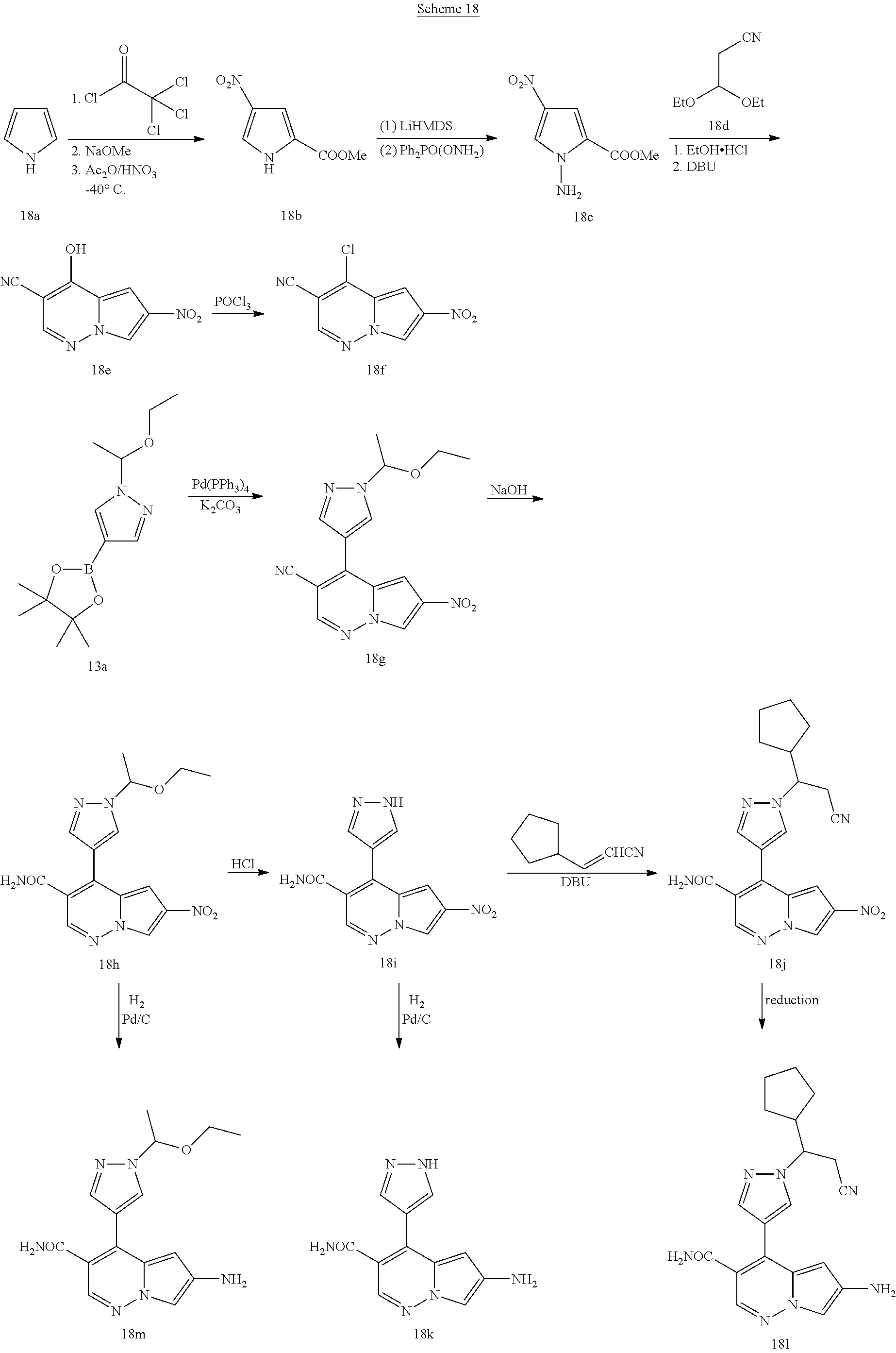

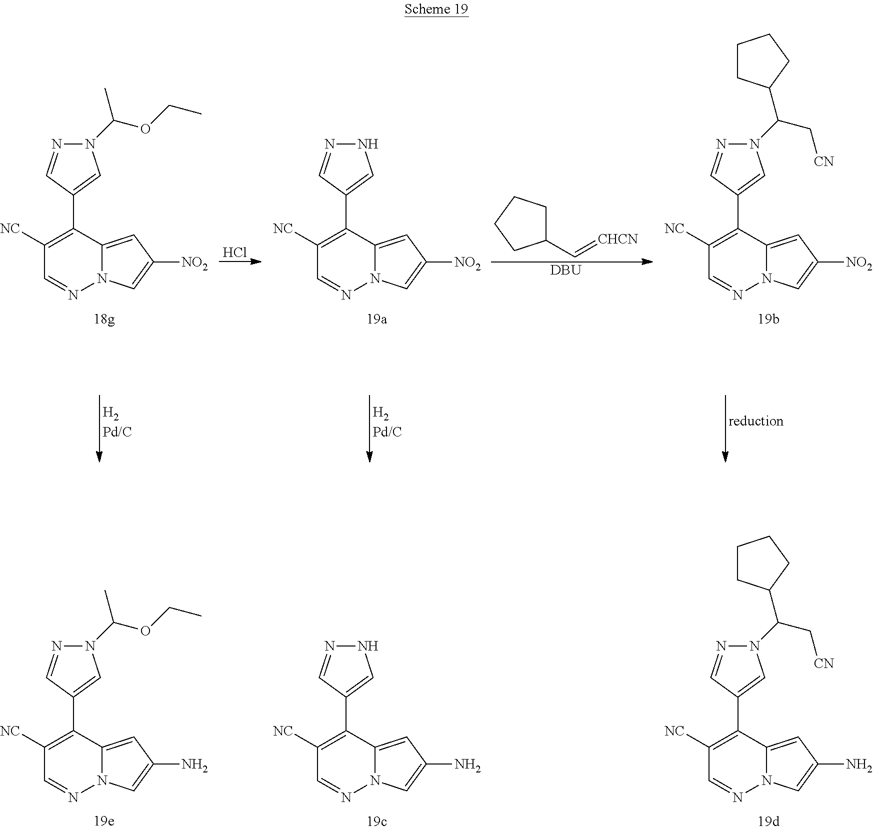

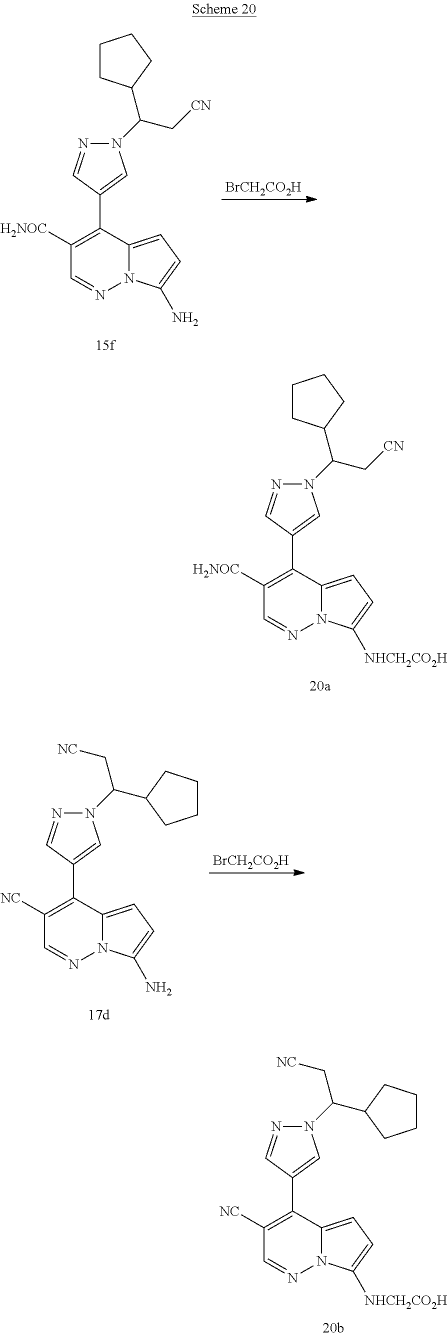

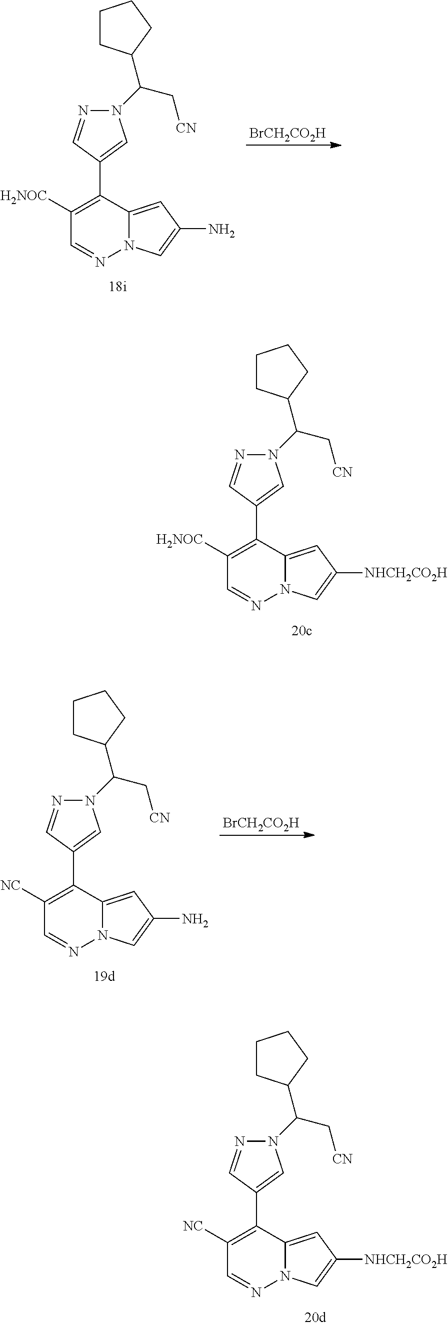

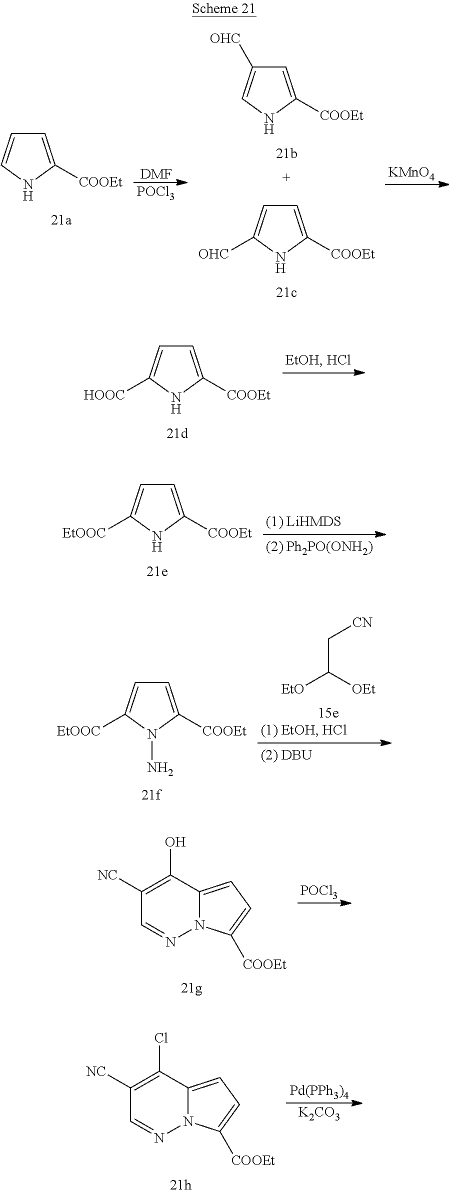

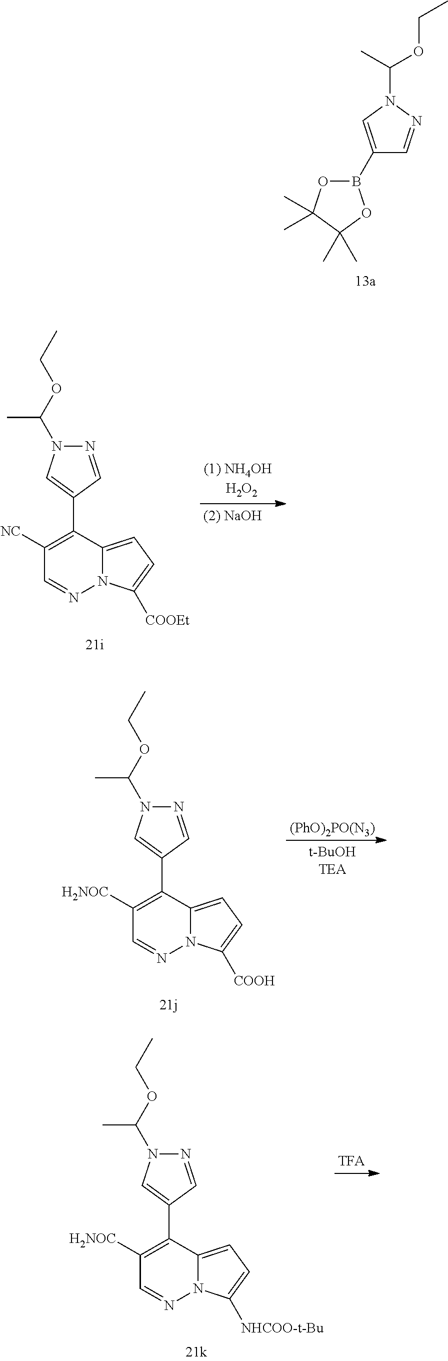

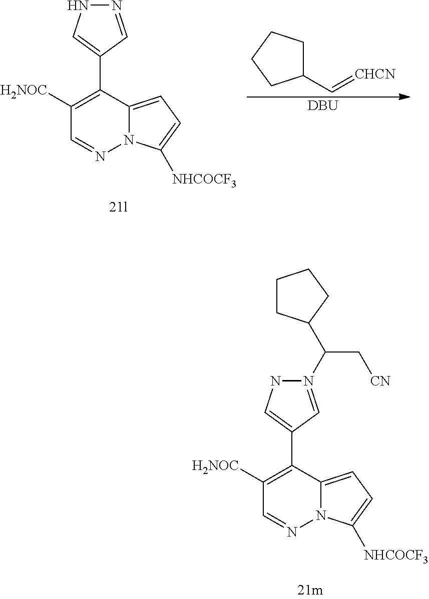

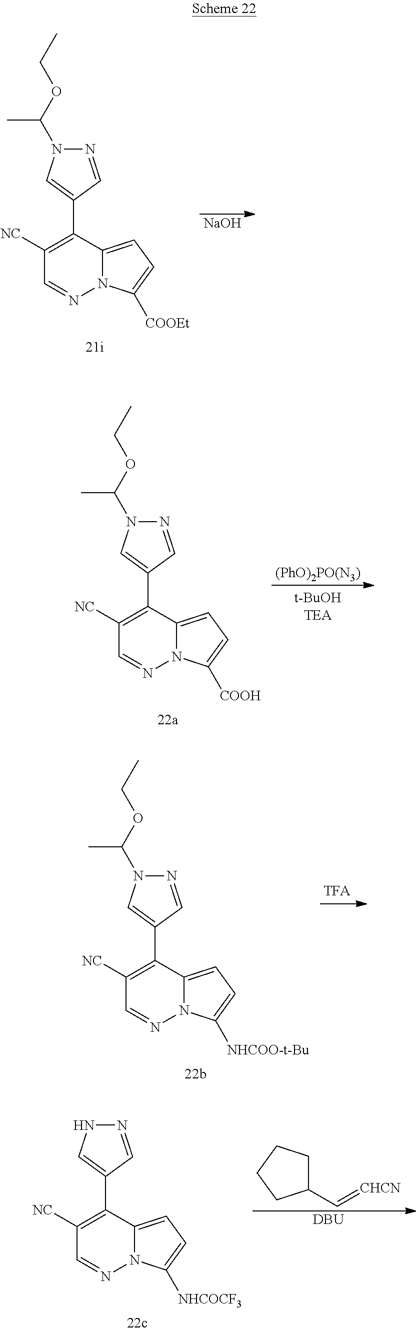

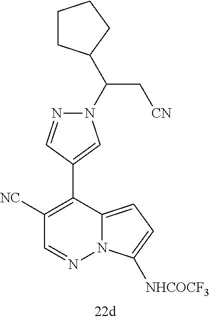

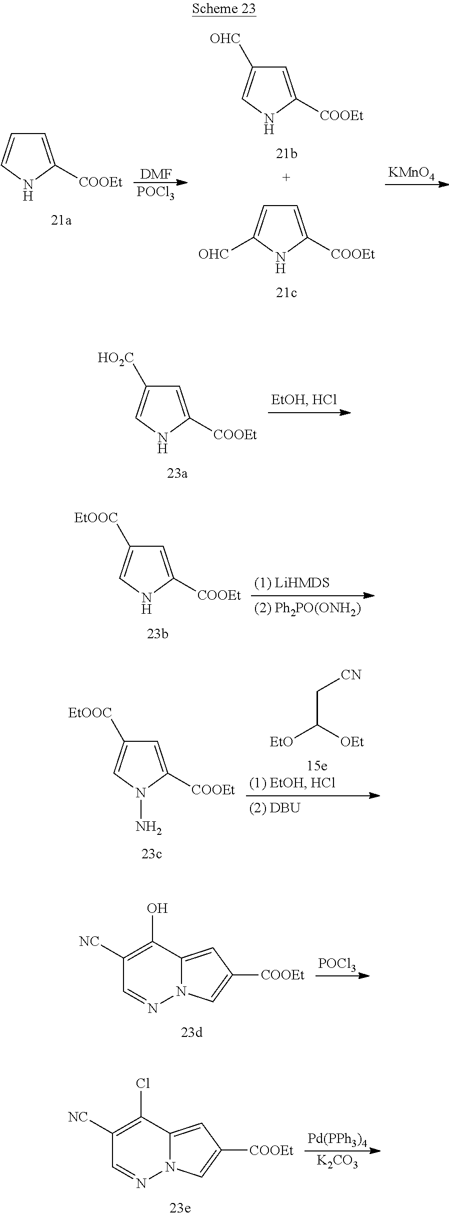

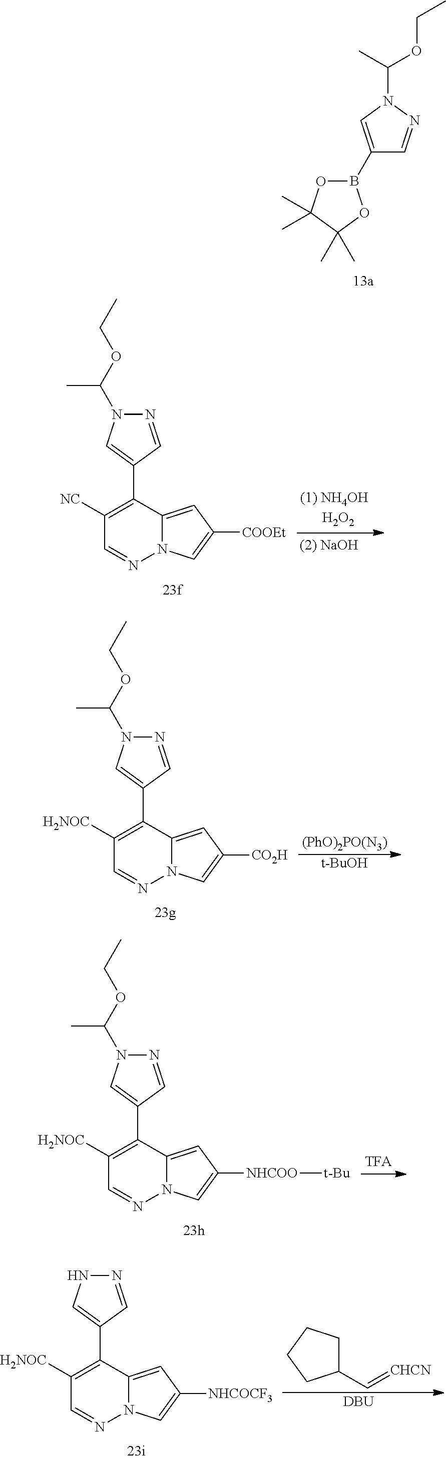

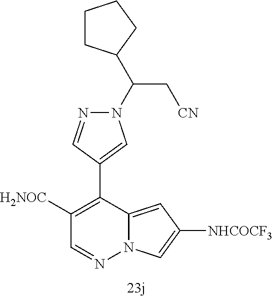

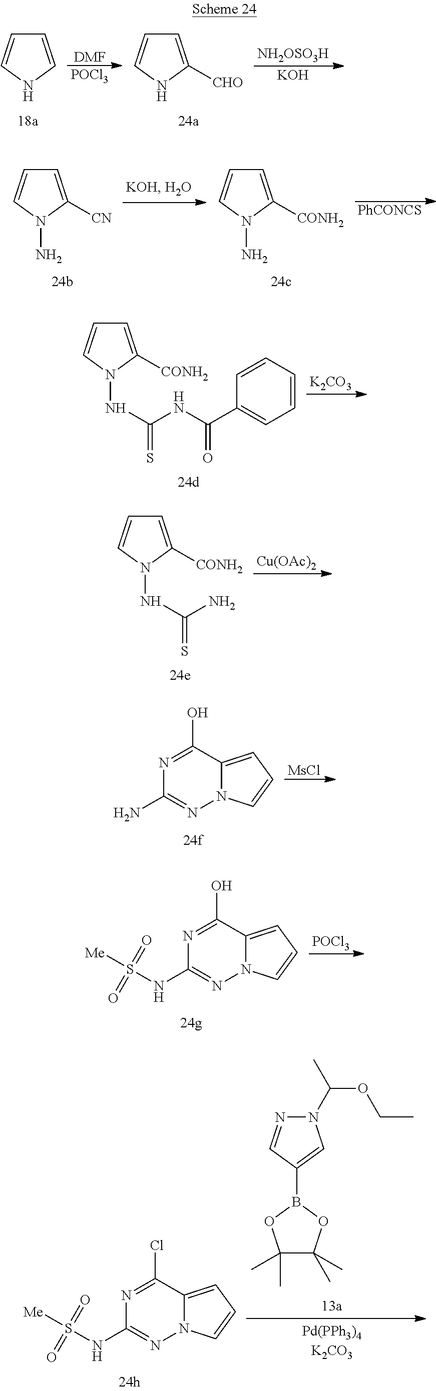

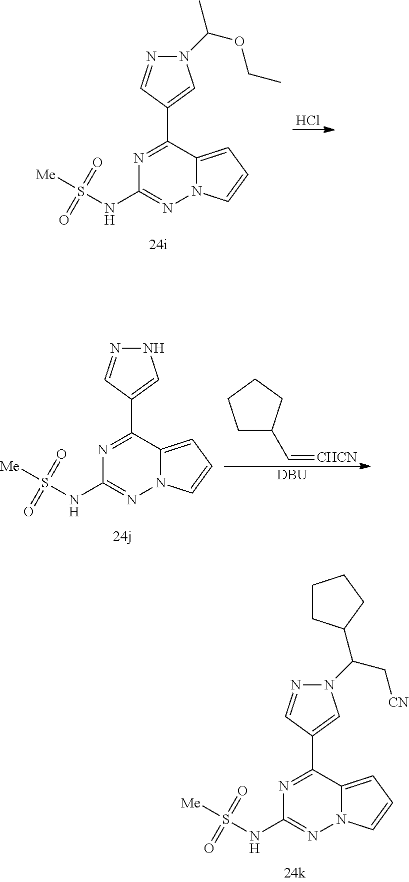

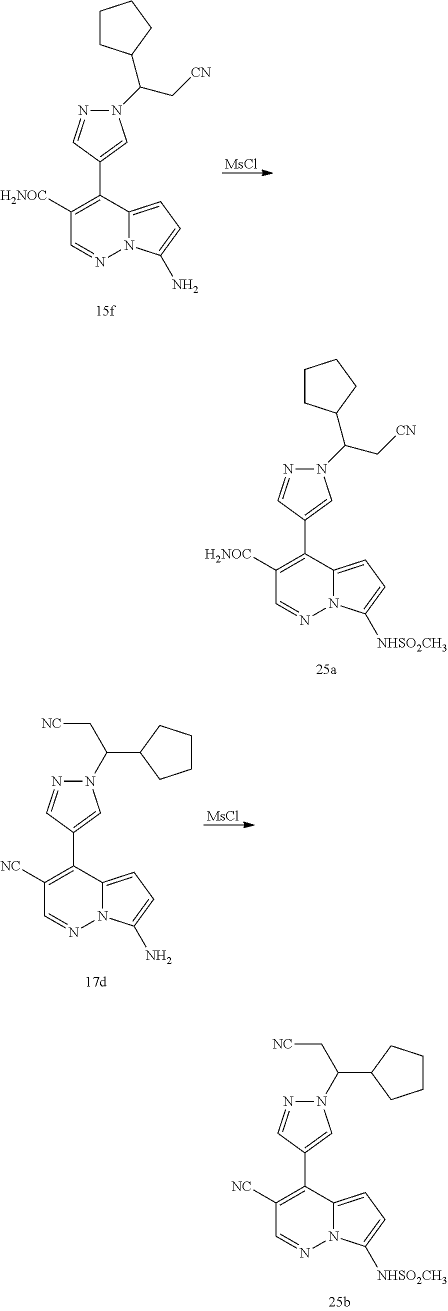

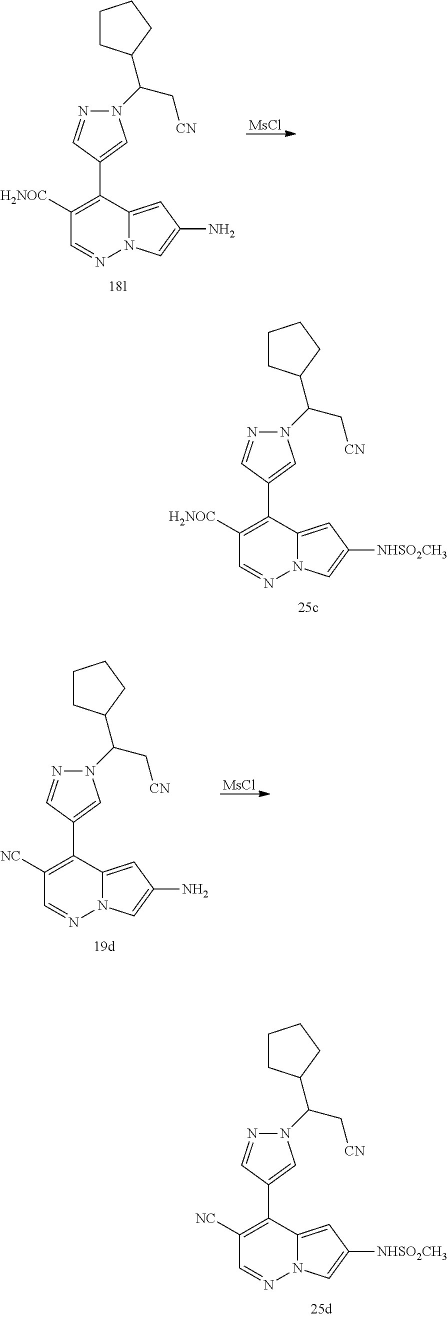

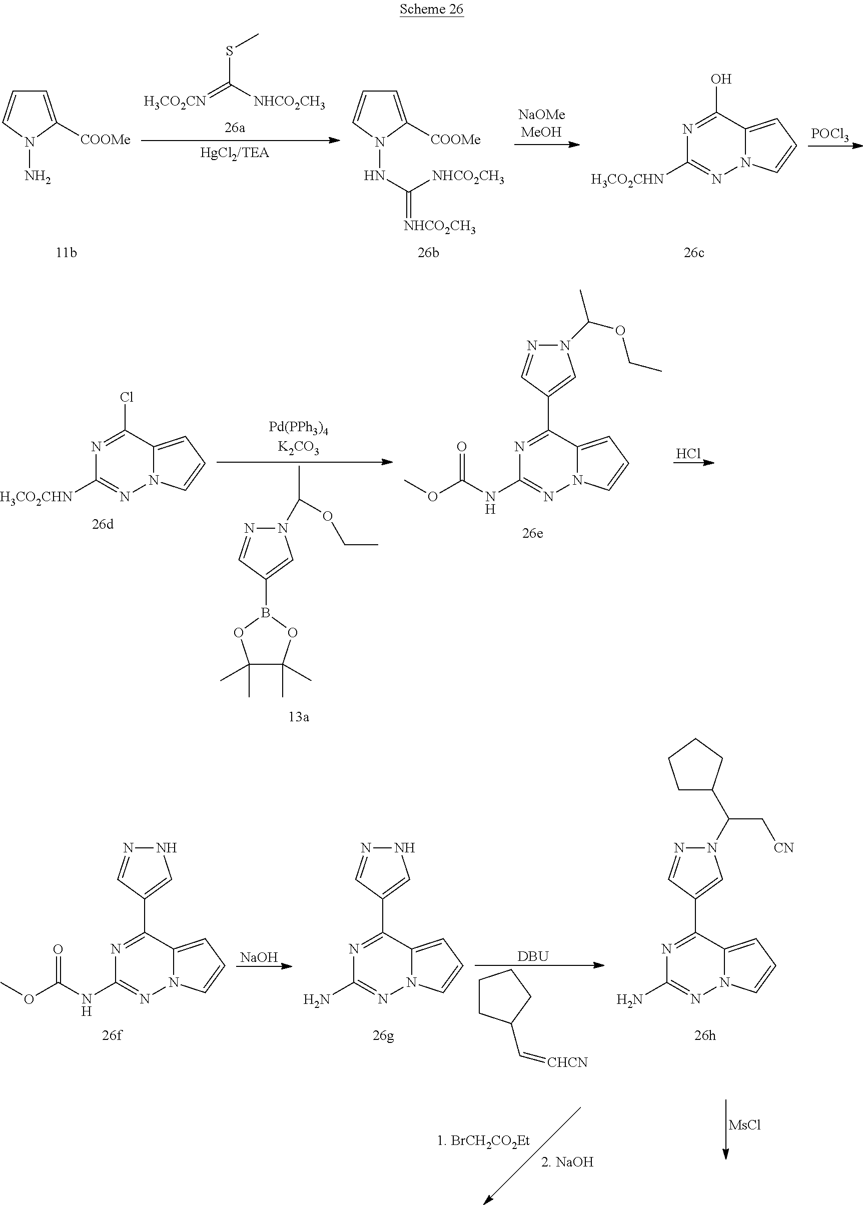

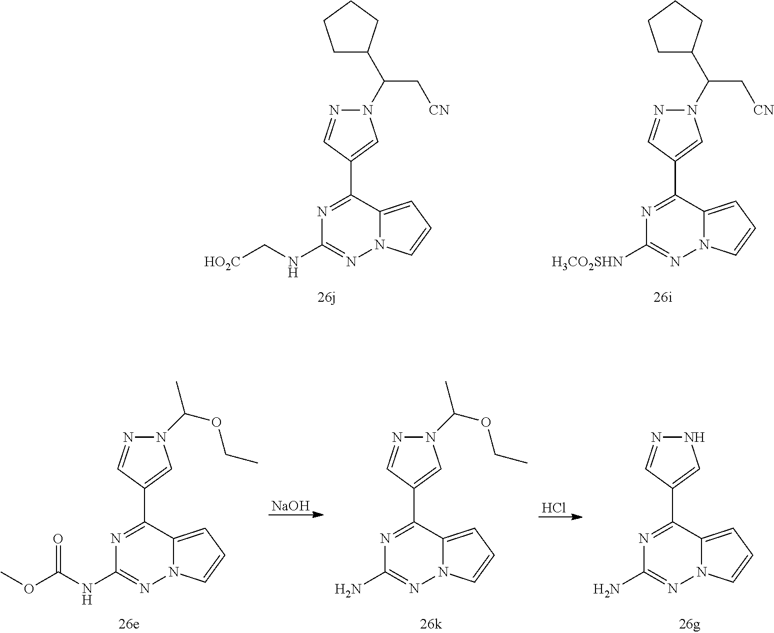

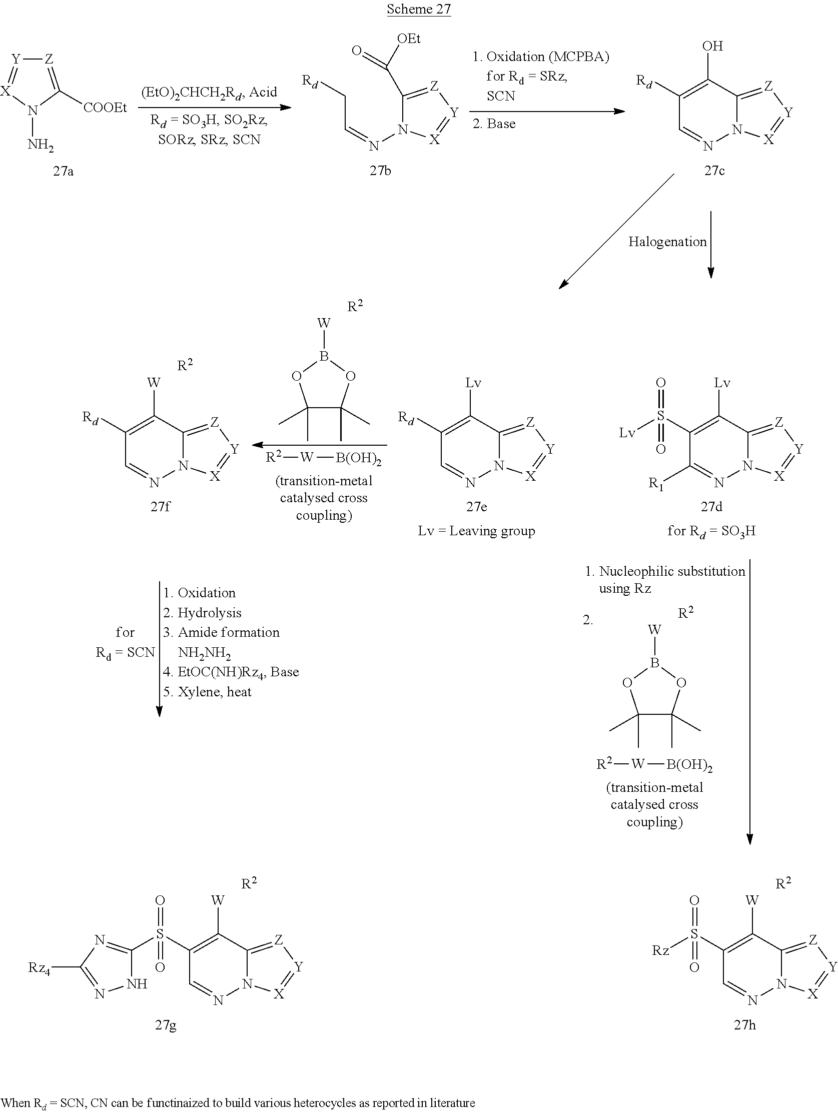

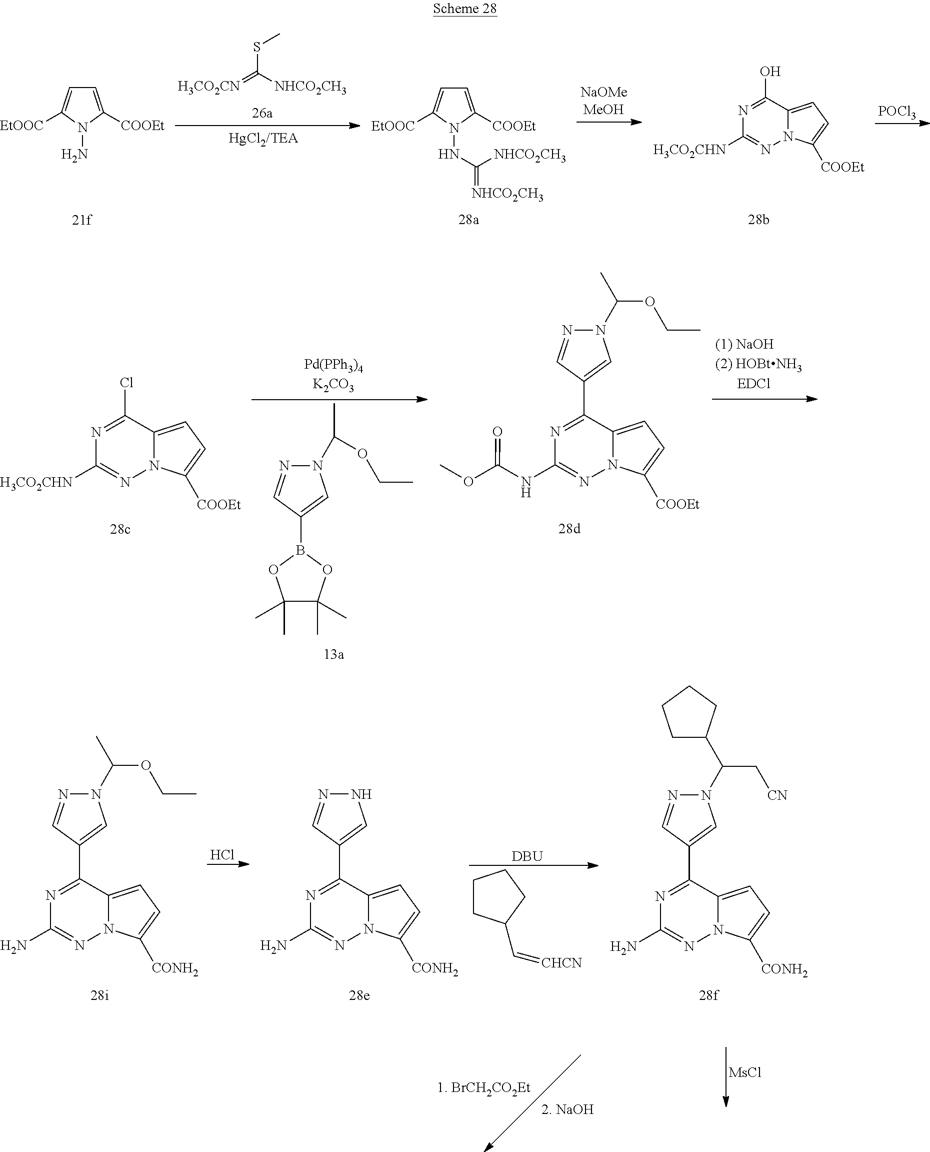

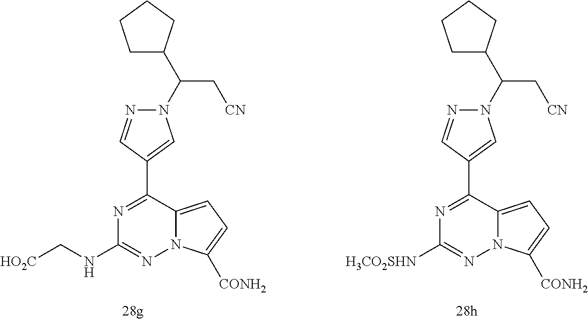

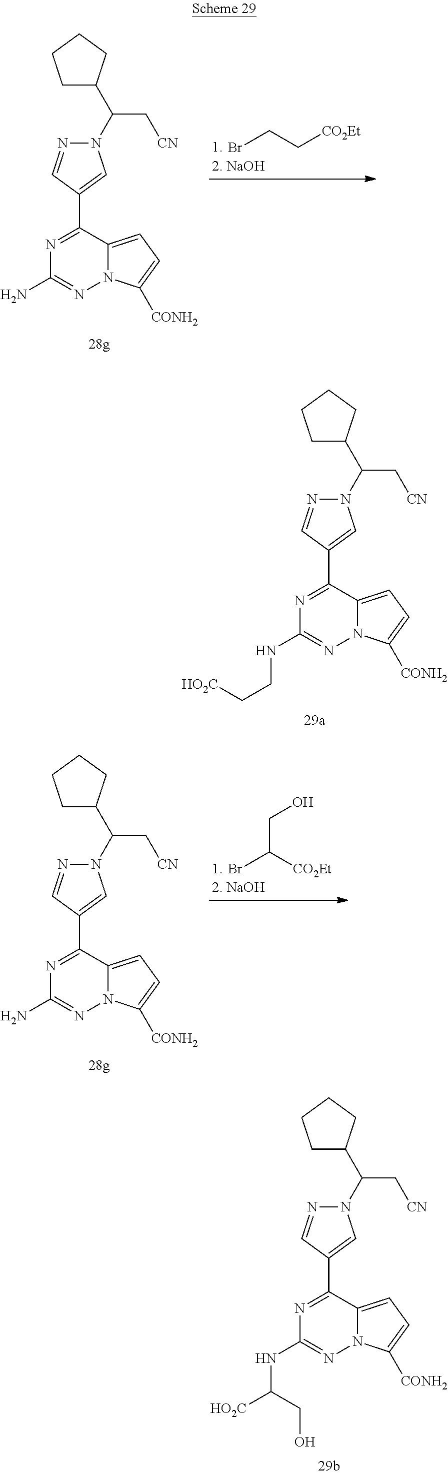 [SMILES: N#CCC(C1CCCC1)n1cc(-c2nc(N)nn3c(C(N)=O)ccc23)cn1 ; CCOC(=O)CCBr ; N#CCC(C1CCCC1)n1cc(-c2nc(NCCC(=O)O)nn3c(C(N)=O)ccc23)cn1 ; N#CCC(C1CCCC1)n1cc(-c2nc(N)nn3c(C(N)=O)ccc23)cn1 ; CCOC(=O)C(Br)CO ; N#CCC(C1CCCC1)n1cc(-c2nc(NC(CO)C(=O)O)nn3c(C(N)=O)ccc23)cn1]

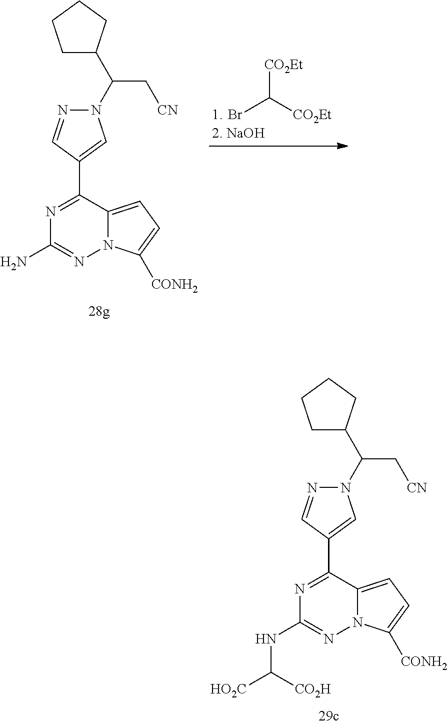 [SMILES: N#CCC(C1CCCC1)n1cc(-c2nc(N)nn3c(C(N)=O)ccc23)cn1 ; CCOC(=O)C(Br)C(=O)OCC ; N#CCC(C1CCCC1)n1cc(-c2nc(NC(C(=O)O)C(=O)O)nn3c(C(N)=O)ccc23)cn1]

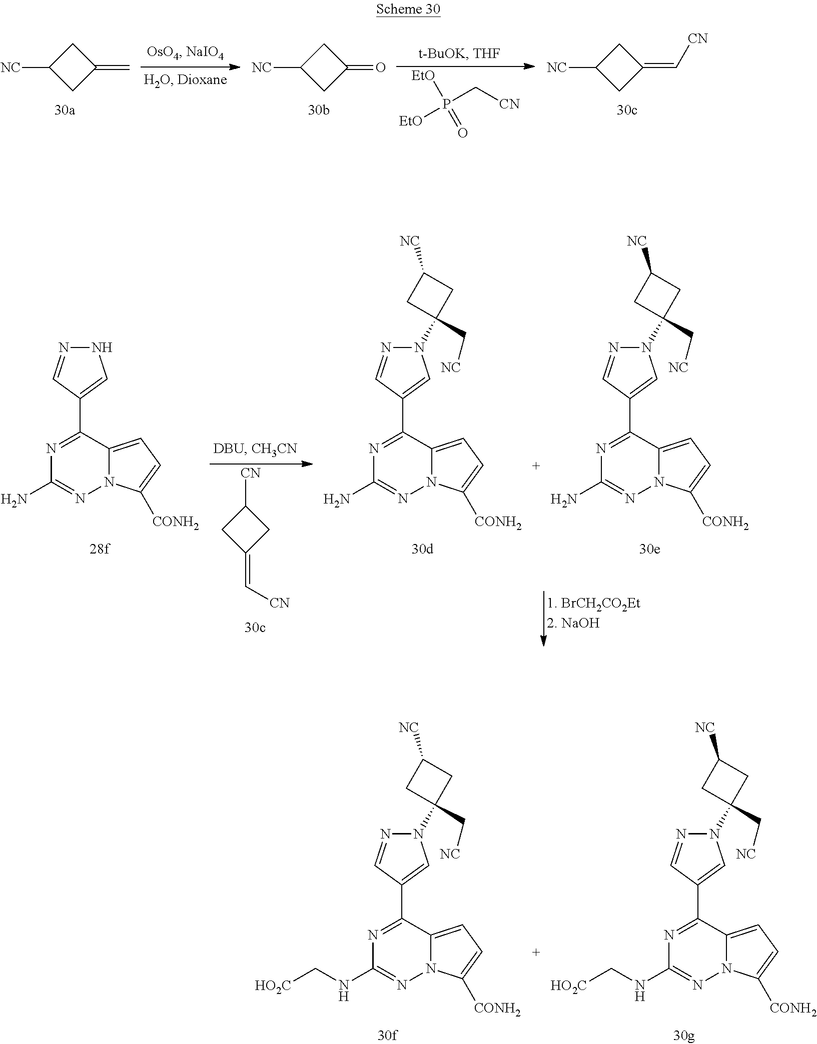

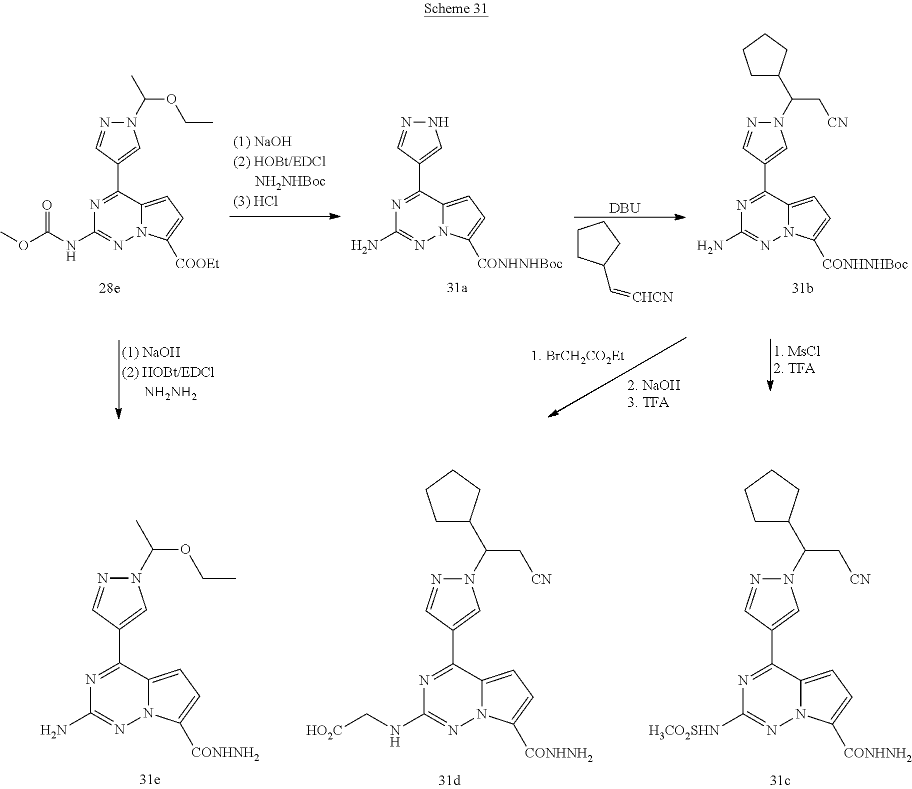

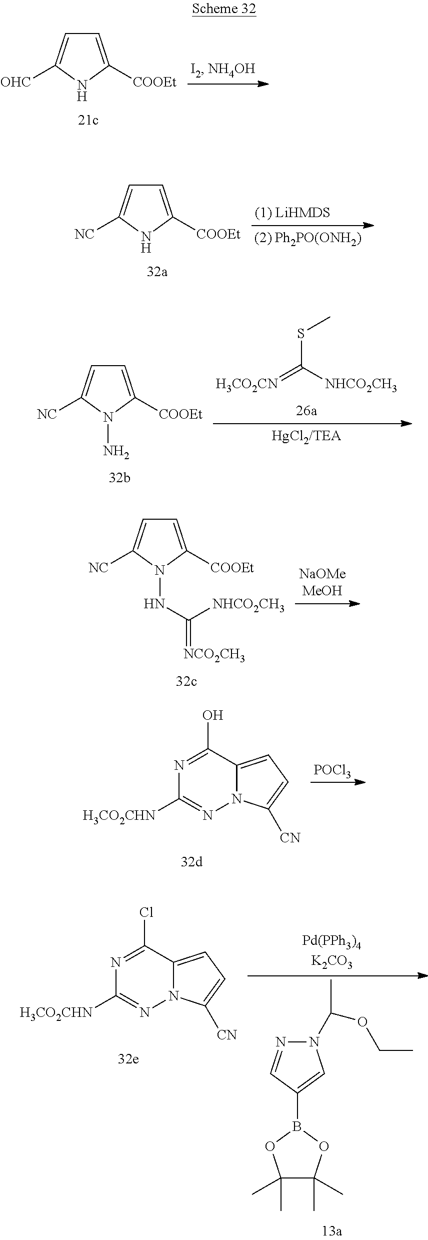

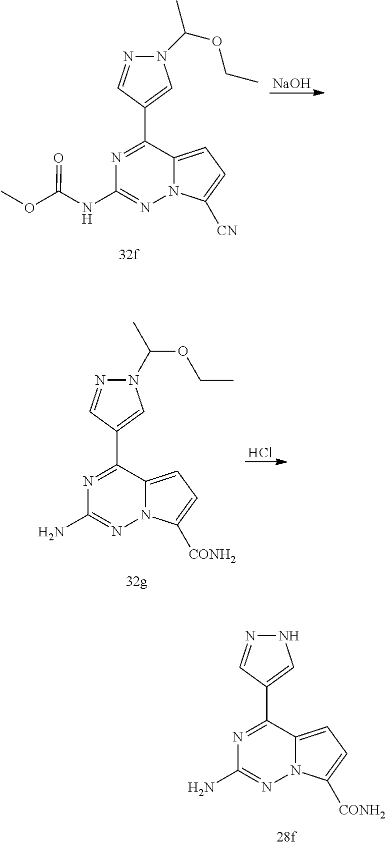

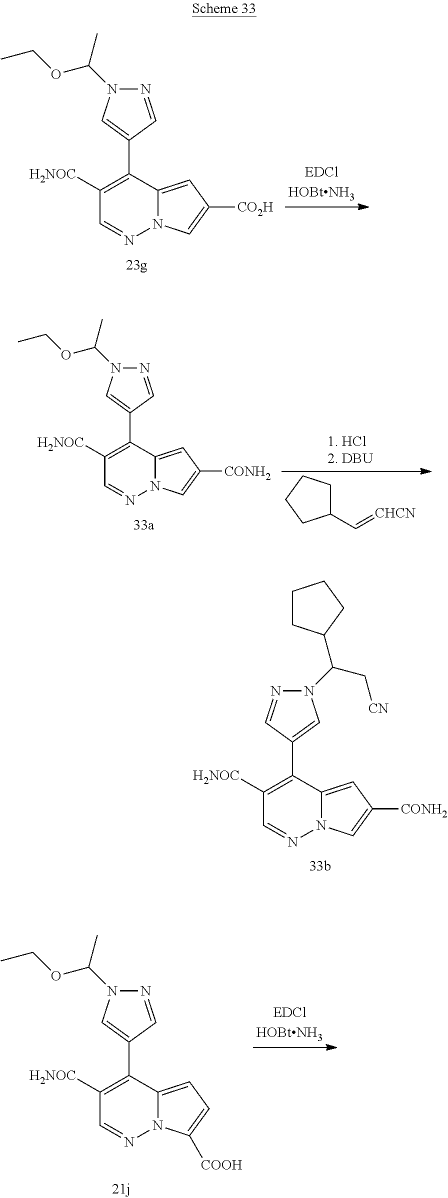

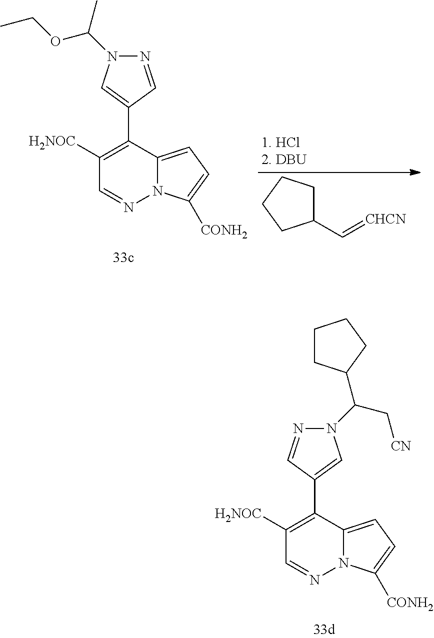

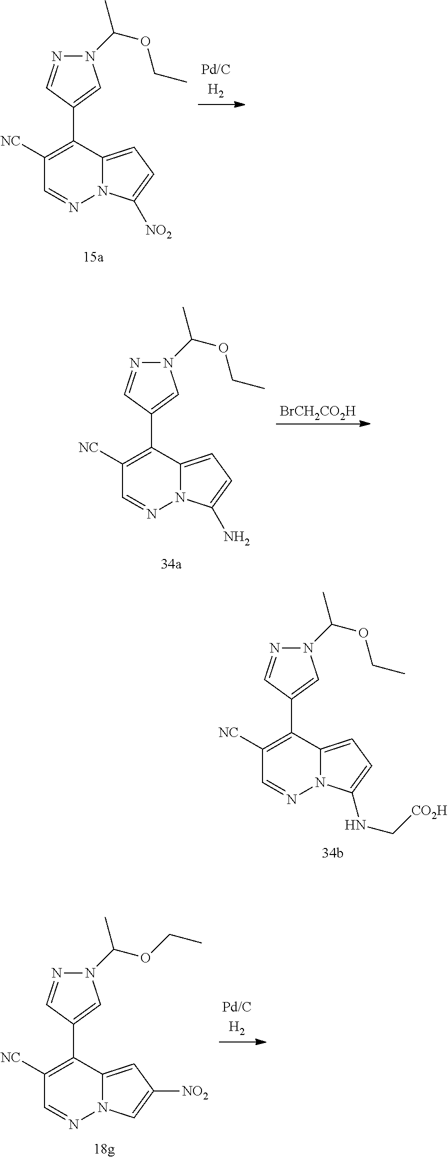

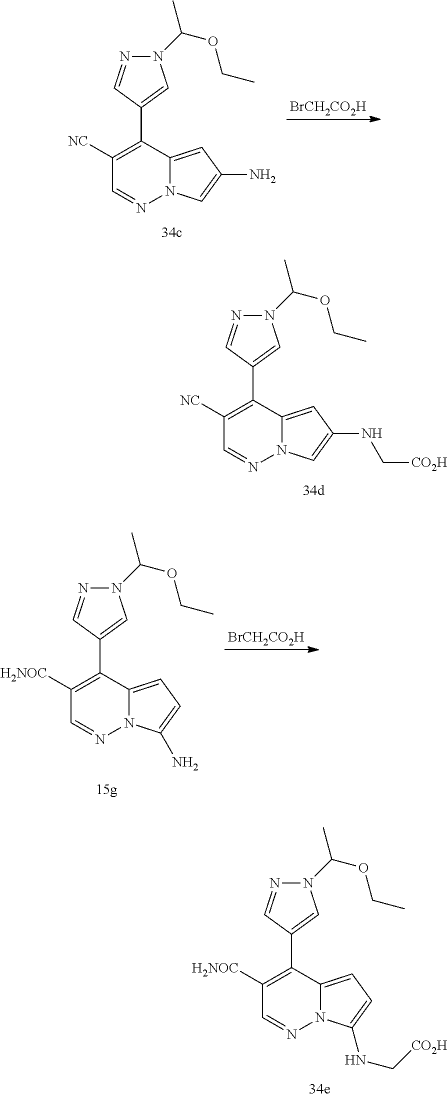

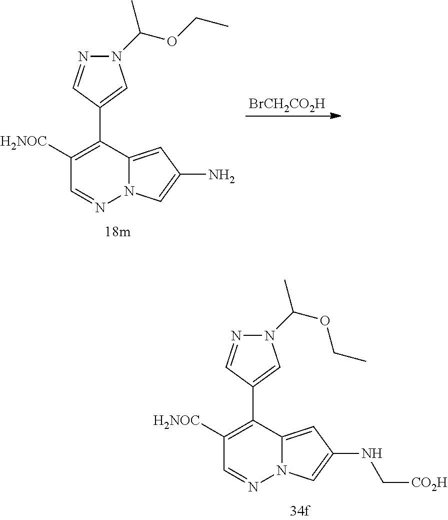

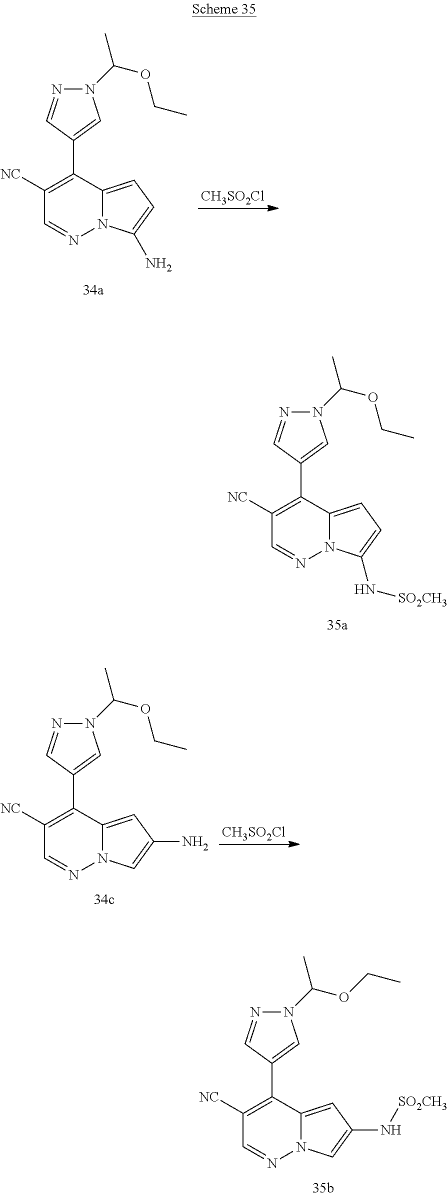

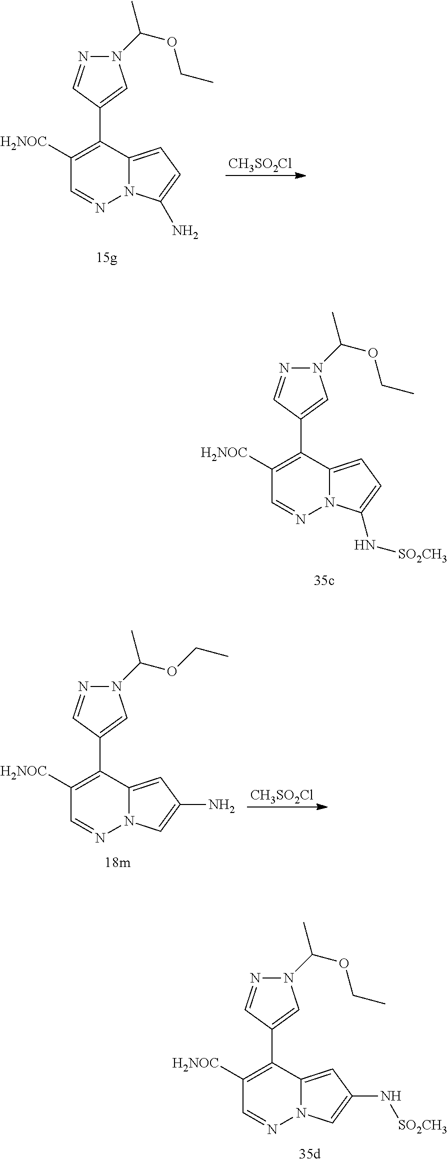

In one embodiment, the invention provides a method for preparing a salt of a compound of formula I, comprising reacting the compound of formula I with an acid under conditions suitable to provide the salt.

In one embodiment, the invention provides a method for preparing a pharmaceutical composition comprising a compound of formula I, or a pharmaceutically acceptable salt thereof, in combination with a pharmaceutically acceptable diluent or carrier, comprising combining the compound of formula I, or the pharmaceutically acceptable salt thereof, with the pharmaceutically acceptable diluent or carrier to provide the pharmaceutical composition.

The compounds of formula I can be formulated as pharmaceutical compositions and administered to a mammalian host, such as a human patient, in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula Ito the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Compounds of the invention can also be administered in combination with other therapeutic agents, for example, other agents that are useful for immunosuppression and the treatment of cancer. Accordingly, in one embodiment the invention also provides a composition comprising a compound of formula I, or a pharmaceutically acceptable salt thereof, at least one other therapeutic agent, and a pharmaceutically acceptable diluent or carrier. The invention also provides a kit comprising a compound of formula I, or a pharmaceutically acceptable salt thereof, at least one other therapeutic agent, packaging material, and instructions for administering the compound of formula I or the pharmaceutically acceptable salt thereof and the other therapeutic agent or agents to an animal to suppress an immune response in the animal.

Compounds of the invention may also be useful in the treatment of other diseases, conditions or disorders associated with the function of kinase such as a Janus kinase (e.g. JAK1, JAK2 or TYK2) including the pathological activation of a kinase such as a Janus kinase (e.g. JAK1, JAK2 or TYK2). Accordingly, in one embodiment the invention provides a compound of formula I for the treatment of a kinase such as a Janus kinase (e.g. JAK1, JAK2 or TYK2) related disease, condition or disorder.

The ability of a compound of the invention to bind to JAK3 may be determined using pharmacological models which are well known to the art, or using Test A described below.

### Test A.

Inhibition constants (ICₛₒs) were determined against JAK3 (JHldomain-catalytic) kinase and other members of the JAK family. Assays were performed as described in Fabian et al. (2005) Nature Biotechnology, vol. 23, p.329 and in Karaman et al. (2008) Nature Biotechnology, vol. 26, p.127. Inhibition constants were determined using 11 point dose response curves which were performed in triplicate. Table 1 shown below lists compounds of the invention and their respective IC₅₀ values.

The ability of a compound of the invention to provide an immunomodulatory effect can also be determined using pharmacological models which are well known to the art. The ability of a compound of the invention to provide an anti-cancer effect can also be determined using pharmacological models which are well known to the art.

The invention will now be illustrated by the following non-limiting Examples.

### EXAMPLE 1

### 3-Cyclopentyl-3-(4-(pyrrolo[1,2-f][1,2,4]triazin-4-yl)-1H-pyrazol-1-yl)propanenitrile (13e)

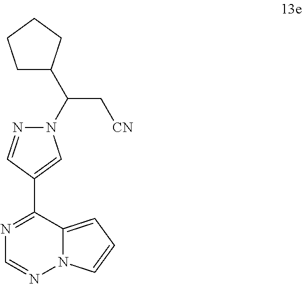 [SMILES: N#CCC(C1CCCC1)n1cc(-c2ncnn3cccc23)cn1]

To a solution of 3-cyclopentyl-3-(4-(pyrrolo [1,2-f][1,2,4]triazin-4-yl)-1H-pyrazol-1-yl)propanal 13d (309 mg, 1 mmol) in THF (3 mL) at room temperature was added a solution of concentrated NH₄OH (2.8 mL) followed by iodine (280 mg). The resulting mixture was stirred at room temperature for 30 min. The reaction was quenched with 25 mL 10% Na₂S₂O₃ aqueous solution and partitioned with ethyl acetate (2×35 mL). The combined organic phases were washed with brine (30 mL), dried over MgSO₄, concentrated to dryness and purified by flash column chromatography to yield 3-cyclopentyl-3-(4-(pyrrolo[1,2-f][1,2,4]triazin-4-yl)-1H-pyrazol-1-yl)propanenitrile 13e (150 mg, 49%) as a colorless syrup. ¹H NMR (300 MHz, DMSO) δ 8.97 (s, 1H), 8.47 (d, J=6.4 Hz, 2H), 8.16 (dd, J=1.3, 26 Hz, 1H), 7.41 (dd, J=1.3, 4.6 Hz, 1H), 7.07 (dd, J=2.6, 4.6 Hz, 1H), 4.57 (m, 1H), 3.25 (d, J=7.0 Hz, 2H), 1.81 (m, 1H), 1.67-1.19 (m, 8H). MS (ES+): 329.1 (M+1); HPLC[ Zorbax SBC3, 3.0×150 mm, 5 μM, with a ZGC SBC3, 2.1×12.5 mm guard cartridge, “A” buffer=(98% of 0.1 M ammonium acetate in 2% acetonitrile); “B” buffer=100% acetonitrile, UV absorbance; Rₜ 13.053=18.307 (94.39%)].

### Preparation of 3-cyclopentyl-3-(4-(pyrrolo[1,2-f][1,2,4]triazin-4-yl)-1H-pyrazol-1-yl)propanal (13d).

To 4-(1H-pyrazol-4-yl)pyrrolo[1,2-d][1,2,4]triazine 13c (0.332 g, 1.5 mmol) in toluene (15 mL) was added 4 nitrobenzoic acid (25 mg) and stirred at room temperature for 10 min, followed by the addition of (E)-3-cyclopentylacrylaldehyde 13f (0.931 g, 7.5 mmol). The resulting mixture was stirred at room temperature overnight and added DBU (224 μL). The reaction mixture was stirred at room temperature for 72 h and concentrated in vacuum to dryness. The residue obtained was purified by flash column chromatography to give 3-cyclopenyl-3-(4-(pyrrolo [1,2-f][1,2,4]triazin-4-yl)-1H-pyrazol-1-yl)propanol 13d (0.318 g, 68%); MS (ES+): 310.1 (M+1).

### EXAMPLE 2

### 4-(1-(1-Ethoxyethyl)-1H-pyrazol-4-yl)pyrrolo[1,2-f][1,2,4]triazine (13b)

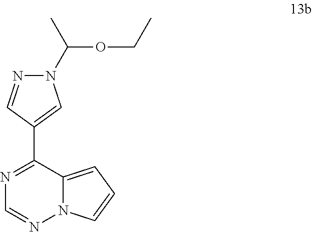 [SMILES: CCOC(C)n1cc(-c2ncnn3cccc23)cn1]

To 4-chloropyrrolo[1,2-f][1,4]triazine 12g (768 mg, 5 mmol), 1,4-dioxane (20 mL), 1-(1-ethoxyethyl)-4-(4,4,5,5-tetramethyl-1,3,2-dioxaborolan-2-yl)-1H-pyrazole 13a (commercially available, 1.6 g, 6 mmol) in water (10 mL) was added and (2.76 g, 20 mmol). The reaction mixture was vacuumed and back-filled with nitrogen. The procedure was repeated three times. Then it was charged with tetrakis(triphenylphosphine) Pd(O) (231 mg, 0.2 mmol). The reaction mixture was flushed with nitrogen again for three times, then stirred at 80° C. under nitrogen for 3 h. The reaction was quenched with water (20 mL) and EtOAc (40 mL). The aqueous layer was separated and extracted with EtOAc (2×30 mL). The organic layers were combined washed with water (40 mL), brine (40 mL), dried over MgSO₄, and concentrated in vacuo. The residue was purified by flash column chromatography (silica gel, eluting with EtOAc/hexane, 0-20%)to furnish 4-(1-(1-ethoxyethyl)-1H-pyrazol-4-yl)pyrrolo[1,2-f][1,2,4]triazine 13b (0.9 mg, 70%) as a yellow oil. ¹H NMR (300 MHz, DMSO) δ 8.96 (d, J=0.5 Hz, 1H), 8.49 (s, 1H), 8.44 (s, 1H), 8.09 (dd, J=1.3, 2.6 Hz, 1H), 7.46 (dd, J=1.3, 4.7 Hz, 1H), 7.07 (dd, J=2.6, 4.6 Hz, 1H), 5.71 (q, J=6.0 Hz, 1H), 3.51 (dq, J=7.0, 9.6 Hz, 1H), 3.27 (dq, J=7.0, 9.6 Hz, 1H), 1.71 (d, J=6.0 Hz, 3H), 1.07 (t, J=7.0 Hz, 3H). MS ES⁺: 258.1, 100%, M+1. HPLC[Zorbax SBC3, 3.0×150 mm, 5 μm, with a ZGC SBC3, 2.1×12.5 mm guard cartridge, “A” buffer=(98% of 0.1 M ammonium acetate in 2% acetonitrile); “B” buffer=100% acetonitrile, UV absorbance t_{R}=16.360, 99.49%; Analysis:Calcd for C₁₃H₁₅N₅O: C, 60.59; H, 5.88; N, 27.22; Found: C, 60.52; H, 5.91; N, 26.97.

### Preparation of 4-chloropyrrolo[1,2-f][1,4]triazine 12g

### Step 1:

To a stirred solution of tert-butyl hydrazinecarboxylate 12a (50 g, 412.37 mmol) and 2,5-dimethoxytetrahydrofuran 12b (54.5 g, 412.37 mmol) in dioxane (300 mL) was added aqueous hydrochloric acid (5 mL, 2N). The reaction was set up using a dean-stark apparatus and heated at 90° C. for 20 h. Reaction mixture was cooled to 20° C., neutralized with saturated sodium bicarbonate (18 mL) and filtered to remove inorganics. The filtrate was concentrated in vacuum and triturated with ether. The solid obtained was collected by filtration to furnish on drying tert-butyl 1H-pyrrol-1-ylcarbamate 12c (43 g, 57.2%) as a yellow brown solid. ¹H NMR (300 MHz, CD₃OD) δ 6.62 (t, J=2.3, 2H), 6.02 (t, J=2.3, 2H), 1.48 (s, 9H); MS (ES⁺): 181.1 (M⁻¹). HPLC (Zorbax SBC3, 3.0×150 mm, 5 μm, with ZGC SBC3, 2.1×12.5 mm guard cartridge. Mobile phase: 0.1 M ammonium acetate/Acetonitrile) Rt=18.44, (100%). Analysis: Calc for C₉H₁₄N₂O₂: C, 59.32; H, 7.74; N, 15.37 Found: C, 59.32; H, 7.65; N, 15.02.

### Step 2:

To a stirred solution of tert-butyl 1H-pyrrol-1-ylcarbamate 12c (40 g, 219.52 mmol), in acetonitrile (350 mL) was added chlorosulfonyl isocyanate (32.62 g, 230.50 mmol) slowly at 0° C. and continued stirring at 0° C. for 30 min. To the solution N,N-dimethyl formamide (40 mL) was added below 5° C. and continued stirring at 0° C. for 1 hr. The reaction mixture was poured into a mixture of crushed ice (1 L) and ethyl acetate (1 L). The layers were separated and the organic layer was washed with water (500 mL), brine (250 mL), dried and concentrated in vacuum to furnish crude (43 g) product. The crude was purified by flash chromatography (silica gel, eluting with ethyl acetate in hexane 0-50%) to afford pure tert-butyl 2-cyano-1H-pyrrol-1-ylcarbamate 12d (30 g, 66%) as a colorless solid. ¹H NMR (300 MHz, DMSO) δ 10.80 (s, 1H, D₂O exchangeable), 7.23 (dd, J=1.7, 2.9, 1H), 6.94 (dd, J=1.7, 4.3, 1H), 6.20 (dd, J=2.9, 4.3, 1H), 1.45 (s, 9H). HPLC (Zorbax SBC3, 3.0×150 mm, 5 μm, with ZGC SBC3, 2.1×12.5 mm guard cartridge. Mobile phase: 0.1 M ammonium acetate/Acetonitrile) Rₜ=16.216, (98.14%). Analysis: Cale for C₁₀H₁₃N₃O₂: C, 57.95; H, 6.32; N, 20.27 Found: C, 58.02; H, 6.45; N, 20.18.

### Step 3:

To a stirred solution of tert-butyl 2-cyano-1H-pyrrol-1-ylcarbamate 12d (5g, 24.12 mmol) in ethyl alcohol (100 ml) was added concentrated aqueous ammonium hydroxide solution (50 mL) at 20° C. followed by hydrogen peroxide (7.4 mL, 72.38 mmol, 30% in water) slowly at 20° C. and stirred at the same temperature for 16 h. Reaction mixture was concentrated in vacuum and diluted with ethyl acetate (150 mL), washed with water (2×50 mL). The aqueous layer was extracted with ethyl acetate (150 mL). The combined ethyl acetate layers were washed with water (100 mL), brine (50 mL), dried, filtered, and concentrated in vacuum. The residue obtained was crystallized from diisopropyl ether and hexane to afford tert-butyl 2-carbamoyl-1H-pyrrol-1-ylcarbamate 12e (4.0 g, 73.6%) as a colorless solid. ¹H NMR (300 MHz, DMSO) δ 9.89 (s, 1H, D₂O exchangeable), 7.31 (d, J=38.5, 1H), 6.84 (dd, J=1.9, 2.8, 2H, 1H is D₂O exchangeable), 6.76 (dd, J=1.9, 4.2, 1H), 5.97 (dd, J=2.8, 4.2, 1H), 1.40 (s, 9H). HPLC (Zorbax SBC3, 3.0×150 mm, 5 μm, with ZGC SBC3, 2.1×12.5 mm guard cartridge. Mobile phase: 0.1 M ammonium acetate/Acetonitrile) Rt=12.817, (97.6861%). Analysis: Cale for C₁₀H₁₅N₃O₃: C, 53.32; H, 6.71; N, 18.65 Found: C, 53.40; H, 6.74; N, 18.55.

### Step 4:

To a solution of tert-butyl 2-carbamoyl-1H-pyrrol-1-ylcarbamate 12e (2g, 8.87 mmol) in dichloromethane (15 mL) was added trifluoroacetic acid (15 mL) at 20° C. and stirred for 30 min. The reaction mixture was concentrated to dryness to remove excess trifluoroacetic acid and diluted with dichloromethane. Triethylorthoformate (30 mL) was added to the residue and was heated to 79° C. overnight. Reaction mixture was concentrated to dryness and triturated with hexanes, the solid obtained was collected by filtration dried in vacuum to give crude pyrrolo[1,2-f][1,2,4]triazin-4-ol 12f (1.1 g, 91%) as a dark brown solid. ¹H NMR (300 MHz, DMSO) δ 11.63 (s, 1H, D₂O exchangeable), 7.83 (d, J=4.0, 1H), 7.59 (dd, J=1.7, 2.6, 1H), 6.89 (dd, J=1.6, 4.3, 1H), 6.54 (dd, J=2.7, 4.3, 1H); MS (ES⁺): 136.2 (M+1). HPLC (SBC3, 3.0×150 mm, 5 μm, with ZGC SBC3, 2.1×12.5 mm guard cartridge. Mobile phase: 0.1 M ammonium acetate/Acetonitrile) Rt=12.817, (95.9%).

### Step 5:

The stirred solution of pyrrolo[1,2-f][1,2,4]triazin-4-ol 12f (1 g, 7.40 mmol), benzyltriethylammonium chloride (3.29 g, 14.80 mmol), and N,N-dimethylaniline (1.35 g, 11.10 mmol) in acetonitrile (25 mL) was heated to 80° C. and at this temperature phosphorous oxy chloride (6.88 g, 44.40 mmol) was added and stirred at 80° C. for 16 h. The reaction was concentrated to remove acetonitrile and phosphorus oxy chloride. The reaction was quenched by adding ice water (20 mL) and extracted with ethyl acetate (2×100 mL). The combined ethyl acetate extracts were washed with hydrochloric acid (1 N, 30 mL) water (50 mL), saturated sodium bicarbonate (1×20 mL), water (50 mL), brine (20 mL) dried and concentrated. The crude residue was purified by flash chromatography [silica gel, eluting with ethyl acetate in hexanes (0 to 5%)] to furnish pure 4-chloropyrrolo[1,2-f][1,2,4]triazine 12g (0.7 g, 61.6%) as a colorless oil, which solidified on standing in refrigerator. ¹H NMR (300 MHz, DMSO) δ 8.44 (s, 1H), 8.27 (dd, J=1.5, 2.5, 1H), 7.12 (qd, J=2.0, 4.6, 2H).

### EXAMPLE 3

### 4-(1H-Pyrazol-4-yl)pyrrolo[1,2-f][1,2,4]triazine (13c)

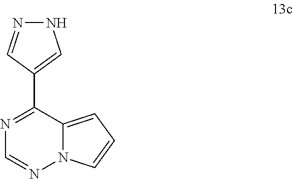 [SMILES: c1cc2c(-c3cn[nH]c3)ncnn2c1]

To a solution of 4-(1-(1-ethoxyethyl)-1H-pyrazol-4-yl)pyrrolo[1,2-f][1,2,4]triazine 13b (0.863 g, 3.36 mmol) in THF (12 mL) was added 2N HCl (5 mL). The reaction mixture was stirred at room temperature overnight and the solvent was removed by evaporation in vacuum. The residue was triturated with ether and the solid obtained was collected by filtration, washed with ether and dried in vacuum to furnish 4-(1H-pyrazol-4-yl)pyrrolo[1,2-f][1,2,4]triazine 13c (0.7 g, 64%) as a yellow solid; mp 245.8° C.; ¹H NMR (300 MHz, DMSO) δ 11.10 (bs, 1H), 8.93 (s, 2H), 8.60 (s, 1H), 8.37 (s, dd, J=1.2, 2.5 Hz, 1H), 7.85, dd, J=1.2, 4.8 Hz, 1H), 7.25 (dd, J=2.5, 4.8 Hz, 1H); MS (ES⁺) 186.0 (M+1); (ES⁻) 184.0, (M−1); Analysis: Calcd for C₉H₇N₅.HCl: C, 48.77; H, 3.64; N, 31.60; Cl, 15.99; Found: C, 48.74; H, 3.69; N, 31.37; Cl, 16.10.

### EXAMPLE 4

### 4-(1H-Pyrazol-4-yl)pyrrolo[1,2-b]pyridazine-3-carboxamide (14c)

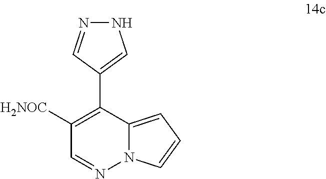 [SMILES: NC(=O)c1cnn2cccc2c1-c1cn[nH]c1]

To a solution of 4-(1-(1-ethoxyethyl)-1H-pyrazol-4-yl)pyrrolo[1,2-b]pyridazine-3-carboxamide 14b (100 mg, 0.33 mmol) in THF (5 mL) was added 1N HCl (aq. 5 mL) and stirred at RT for 5 h. The reaction mixture was neutralized with 6N NaOH and diluted with water (30 mL). The reaction mixture was extracted with ethyl acetate (60 mL, 30mL) The combined ethyl acetate layers were washed with brine (30 mL) dried over MgSO₄ filtered and concentrated in vacuum. The residue obtained was purified by flash column chromatography [silica gel 4 g, eluting with hexanes/10% MeOH in ethyl acetate,1:0 to 1:4, (R_{f}=0.24 with hexanes/10% MeOH in ethyl acetate=1:4)] to furnish 4-(1H-pyrazol-4-yl)pyrrolo[1,2-b]pyridazine-3-carboxamide 14c (32 mg, 43%) as a yellow solid. ¹H NMR (300 MHz, DMSO) δ 13.32 (s, 1H), 8.21 (bs, 1H), 8.12 (s, 1H), 7.96 (bs, 1H), 7.93 (dd, J=1.6, 2.7, 1H), 7.74 (s, 1H), 7.55 (s, 1H), 6.92 (dd, J=2.7, 4.2, 1H), 6.75 (dd, J=1.6, 4.4, 1H). MS (ES): 225.9 (M-1).

### EXAMPLE 5

### 4-(1-(1-Ethoxyethyl)-1H-pyrazol-4-yl)pyrrolo[1,2-b]pyridazine-3-carbonitrile (14a)

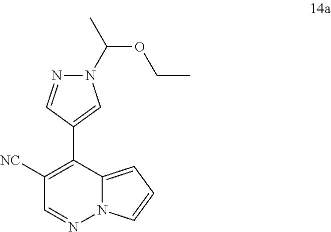 [SMILES: CCOC(C)n1cc(-c2c(C#N)cnn3cccc23)cn1]

To a mixture of 4-chloro-[1,2-b]pyridazine-3-carbonitrile 11d (1.0 g, 5.63 mmol), 1,4-dioxane (20 mL), 1-(1-ethoxyethyl)-4-(4,4,5,5-tetramethyl-1,3,2-dioxaborolan-2-yl)-1H-pyrazole 13a (commercially available, 1.80 g, 6.76 mmol) in 1,4-dioxane (15 mL) and water (7.5 mL) under argon was added K₂CO₃ (3.12 g, 22.58 mmol) Pd(PPh₃)₄ (252 mg, 0.22 mmol) and heated at 85° C. for 3 h. The reaction mixture was cooled to RT, diluted with ethyl acetate (200 mL), washed with water (100 mL) and brine (50 mL), dried over MgSO₄ filtered and concentrated in vacuum. The residue obtained was purified by flash column chromatography [silica gel 40 g, eluting with hexanes/ethyl acetate, 1:0 to 5:1, (R_{f}=0.46 with hexanes/ethyl acetate=4:1)] to afford 4-(1-(1-ethoxyethyl)-1H-pyrazol-4-yl)pyrrolo[1,2-b]pyridazine-3-carbonitrile 14a (1.39 g, 88%, yellow solid); ¹H NMR (300 MHz, DMSO-d₆): δ 8.78 (d, J=0.7 Hz, 1H), 8.49 (s, 1H), 8.27 (s, 1H), 8.19 (dd, J=1.6, 2.5 Hz, 1H), 7.17-7.10 (m, 2H), 5.74 (q, J=5.9 Hz, 1H), 3.60-3.46 (m, 1H), 3.34-3.24 (m, 1H), 1.68 (d, J=6.0 Hz, 3H), 1.08 (t, J=7.0 Hz, 314); MS (ES⁺): 282.1 (M+H).

### Preparation of 4-chloro-[1,2-b]pyridazine-3-carbonitrile 11d

### Step 1:

To a solution of ethyl pyrrole-2-carboxylate IIa (5.0 g, 35.21 mmol) in DMF (300 mL) cooled to −10° C. was added LiHMDS (1 M in THF, 42.3 mL) and stirred at −10° C. for 15 min.

The reaction mixture was treated with O-(diphenylphosphoryl)hydroxylamine (15 g, 64.32 mmol) at −10° C. and stirred at RT for 20 h. The reaction mixture was diluted with ethyl acetate (800 mL), washed with water (2×400 mL), brine (200 mL), dried over MgSO₄ and filtered. The filtrate was concentrated in vacuum and the residue obtained was purified by flash column chromatography [silica gel 200 g, eluting with hexanes/ethyl acetate, 1:0 to 4:1, (R_{f}=0.46 with hexanes/ethyl acetate=4:1)] to afford ethyl 1-amino-1H-pyrrole-2-carboxylate 11b (3.87 g, 71%) as a light yellow oil. ¹H NMR (300 MHz, DMSO-d₆): δ 7.01 (t, J=2.3 Hz, 1H), 6.70 (dd, J=2.0, 4.3 Hz, 1H), 6.26 (s, 2H), 5.97 (dd, J=2.6, 4.3 Hz, 1H), 4.22 (q, J=7.1 Hz, 2H), 1.27 (t,

J=7.1 Hz, 3H).

### Step 2:

To a solution of ethyl 1-amino-1H-pyrrole-2-carboxylate 11b (3.0 g, 19.46 mmol) in EtOH (100 mL) was added 3,3-diethoxypropanenitrile (25 mL, 95%, 158.23 mmol), 1N HCl (aq. 5 mL) and heated at reflux for 18 h. The reaction mixture was cooled to RT, treated with DBU (32.5 mL, 213.18 mmol), and heated at reflux for 1 h. The reaction mixture was concentrated in vacuo and the residue obtained was diluted with ethyl acetate (300 mL), extracted with water (200 mL, 150 mL). The aqueous layers were combined acidified with 4 N HCl to pH=1 and extracted with chloroform/methanol (3:1, 4×200 mL). The combined chloroform layers were dried over MgSO₄ filtered and concentrated in vacuum. The residue obtained was purified by flash column chromatography [silica gel 120 g, eluting with hexanes/ethyl acetate/MeOH, 1:1:0 to 2:2:1 (R_{f}=0.35 with hexanes/ethyl acetate/MeOH=2:2:1)] to furnish 4-hydroxy-[1,2-b]pyridazine-3-carbonitrile 11c (1.44 g, 47%) as a brown solid; ¹H NMR (300 MHz, DMSO-d₆): δ 8.16 (s, 1H), 7.90 (dd, J=1.6, 2.6 Hz, 1H), 7.08 (dd, J=1.6, 4.5 Hz, 1H), 6.80 (dd, J=2.6, 4.5 Hz, 1H); MS (ES): 157.8 (M−H).

### Step 3:

To a solution 4-hydroxy-[1,2-b]pyridazine-3-carbonitrile 11c (1.26 g, 7.91 mmol) in acetonitrile (40 mL) was added benzyltriethylammonium chloride (3.68 g, 15.83 mmol), N,N-dimethylaniline (1.6 mL, 12.50 mmol) and heated to 80° C. To the hot reaction mixture was added dropwise POCl₃ (4.4 mL, 47.59 mmol) and continue heating at 80° C. for 1 h. The reaction mixture was cooled to room temperature and concentrated in vacuum to dryness. The residue obtained was dissolved in chloroform (400 mL), washed with 1N NaHCO₃ (200 mL), water (200 mL), brine (100 mL), dried over MgSO₄ filtered and concentrated in vacuum to dryness. The residue obtained was purified by flash column chromatography [silica gel 50 g, eluting with hexanes/ethyl acetate, 1:0 to 6:1, (R_{f}=0.57 with hexanes/ethyl acetate=6:1)] to furnish 4-chloro-[1,2-b]pyridazine-3-carbonitrile 11d (1.075 g, 77%) as a yellow solid. MP 115.1° C.; ¹H NMR (300 MHz, DMSO-d₆): δ 8.57 (s, 1H), 8.31 (dd, J=1.5, 2.6 Hz, 1H), 7.22-7.18 (m, 1H), 7.13 (dd, J=1.5, 4.6 Hz, 1H).

### EXAMPLE 6

### 4-(1-(1-Ethoxyethyl)-1H-pyrazol-4-yl)pyrrolo[1,2-b]pyridazine-3-carboxamide (14b)

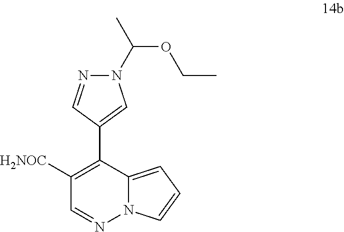 [SMILES: CCOC(C)n1cc(-c2c(C(N)=O)cnn3cccc23)cn1]

To a solution of 4-(1-(1-ethoxyethyl)-1H-pyrazol-4-yl)pyrrolo [1,2-b]pyridazine-3-carbonitrile 14a (1.36 g, 4.83 mmol) in THF (40 mL) and MeOH (32 mL) was added 6 N aqueous NaOH (30 mL) and heated at 70° C. for 5 h. Additional 6 N NaOH (38 mL) and MeOH (38 mL) was added and continued heating at 70° C. for additional 4 h. The reaction mixture was cooled to room temperature and neutralized with 4 N HCl. The reaction mixture extracted with ethyl acetate (300mL, 100 mL). The combined ethyl acetate layers were washed with brine (100 mL) dried over MgSO₄ filtered concentrated in vacuum. The residue obtained was purified by flash column chromatography [silica gel 24 g, eluting with hexanes/10% MeOH in ethyl acetate,1:0 to 1:1 (R_{f}=0.34 with hexanes/ethyl acetate/MeOH=10:10:1)] to afford 4-(1-(1-ethoxyethyl)-1H-pyrazol-4-yl)pyrrolo[1,2-b]pyridazine-3-carboxamide 14b (0.777 g, 54%) as a yellow solid. ¹H NMR (300 MHz, DMSO-d₆): δ 8.40 (d, J=0.6 Hz, 1H), 8.15 (s, 1H), 7.95 (dd, J=1.5, 2.7 Hz, 1H), 7.93 (s, 1H), 7.77 (s, 1H), 7.59 (s, 1H), 6.94 (dd, J=2.8, 4.3 Hz, 1H), 6.75 (dd, J=1.5, 4.4 Hz, 1H), 5.66 (q, J=6.0 Hz, 1H), 3.56-3.39 (m, 1H), 3.30-3.17 (m, 1H), 1.65 (d, J=6.0 Hz, 3H), 1.07 (t, J=7.0 Hz, 3H); MS (ES⁺): 322.1 (M+Na).

### EXAMPLE 7

### 4-(1-(2-Cyano-1-cyclopentylethyl)-1H-pyrazol-4-yl)pyrrolo[1,2-b]pyridazine-3-carboxamide (14d)

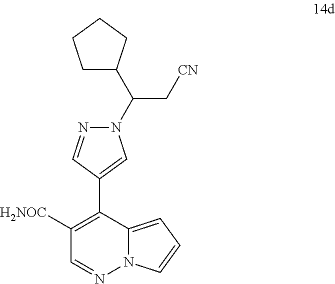 [SMILES: N#CCC(C1CCCC1)n1cc(-c2c(C(N)=O)cnn3cccc23)cn1]

To a solution of 4-(1H-pyrazol-4-yl)pyrrolo[1,2-b]pyridazine-3-carboxamide 14c (30 mg, 0.13 mmol) in acetonitrile was added 3-cyclopentylacrylonitrile (0.33 mol), DBU (0.020 mL, 0.13 mmol) and heated at 50° C. for 3 h. The reaction mixture was concentrated in vacuum and the residue obtained was purified by flash column chromatography [silica gel 4g, eluting with hexanes/10% MeOH in ethyl acetate,1:0 to 1:1, (R_{f}=0.40 with hexanes/ethyl acetate/methanol=10:10:1)] to give 4-(1-(2-cyano-1-cyclopentylethyl)-1H-pyrazol-4-yl)pyrrolo[1,2-b]pyridazine-3-carboxamide 14d, (38 mg, 84%) as a yellow film. ¹H NMR (300 MHz, DMSO) δ 8.43 (bs, 1H), 8.15 (s, 1H), 7.97-7.92 (m, 2H), 7.75 (s, 1H), 7.58 (s, 1H), 6.95 (dd, J=2.7, 4.4 Hz, 1H), 6.74 (dd, J=1.5, 4.4 Hz, 1H), 4.55 (td, J=4.5, 9.2, 1H), 3.26-3.10 (m, 2H), 2.47-2.30 (m, 1H), 1.87-1.72 (m, 1H), 1.67-1.24 (m, 7H); MS (ES⁺): 371.1 (M+Na).

### EXAMPLE 8

### 4-(1-(1-Ethoxyethyl)-1H-pyrazol-4-yl)-6-nitropyrrolo[1,2-b]pyridazine-3-carboxamide (18h)

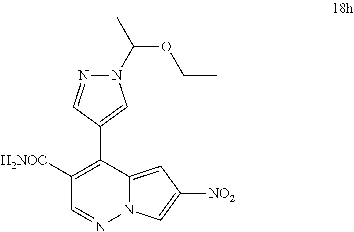 [SMILES: CCOC(C)n1cc(-c2c(C(N)=O)cnn3cc([N+](=O)[O-])cc23)cn1]

To a solution of 4-(1-(1-ethoxyethyl)-1H-pyrazol-4-yl)-6-nitropyrrolo[1,2-b]pyridazine-3-carbonitrile 18g (165 mg, 0.51 mmol) in THF (15 mL) and MeOH (12.5 mL) was added 6 N NaOH (aq. 2.5 mL) and stirred at RT for 14 h. Additional 6 N NaOH (11 mL) was added and the reaction was continued stirring at RT again for 24 h. The reaction mixture was neutralized with 4 N HCl and extracted with ethyl acetate (200 mL). The ethyl acetate layer was washed with brine (50 mL) dried over MgSO₄ filtered and concentrated in vacuum. The residue obtained was purified by flash column chromatography [silica gel 4 g, eluting with hexanes/10% MeOH in ethyl acetate,1:0 to 1:1, (R_{f}=0.35 with hexanes/ethyl acetate/MeOH=10:10:1)] to furnish 4-(1-(1-ethoxyethyl)-1H-pyrazol-4-yl)-6-nitropyrrolo[1,2-b]pyridazine-3-carboxamide 18h (46 mg, 26%) as a yellow solid; ¹H NMR (300 MHz, DMSO-d₆): δ 8.93 (d, J=2.0 Hz, 1H), 8.57 (s, 1H), 8.45 (s, 1H), 8.00 (s, 1H), 7.98 (s, 1H), 7.79 (s, 1H), 7.32 (d, J=2.0 Hz, 1H), 5.69 (q, J=5.9 Hz, 1H), 3.56-3.45 (m, 1H), 3.28-3.23 (m, 1H), 1.66 (d, J=5.9 Hz, 3H), 1.07 (t, J=7.0 Hz, 3H); MS (ES⁻): 343.27 (M−1).

### EXAMPLE 9

### 4-(1-(1-Ethoxyethyl)-1H-pyrazol-4-yl)-6-nitropyrrolo[1,2-b]pyridazine-3-carbonitrile (18g)

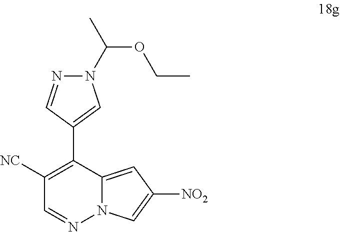 [SMILES: CCOC(C)n1cc(-c2c(C#N)cnn3cc([N+](=O)[O-])cc23)cn1]

To a mixture of 4-chloro-6-nitropyrrolo[1,2-b]pyridazine-3-carbonitrile 18f (0.13 g, 0.58 mmol), 1-(1-ethoxyethyl)-4-(4,4,5,5-tetramethyl-1,3,2-dioxaborolan-2-yl)-1H-pyrazole 13a (commercially available, 0.15 g, 0.695 mmol) in 1,4-dioxane (5 mL) and water (2.5 mL) under argon was added K₂CO₃ (0.312 g, 2.32 mmol) Pd(PPh₃)₄ (26 mg, 0.023 mmol) and heated at 85° C. for 4 h. The reaction mixture was cooled to RT, diluted with ethyl acetate (100 mL), washed with water (50 mL) and brine (50 mL), dried over MgSO₄ filtered and concentrated in vacuum. The residue obtained was purified by flash column chromatography [silica gel 4 g, eluting with hexanes/ethyl acetate, 1:0 to 5:1, (R_{f}=0.24 with hexanes/ethyl acetate=5:1)] to afford 4-(1-(1-ethoxyethyl)-1H-pyrazol-4-yl)-6-nitropyrrolo[1,2-b]pyridazine-3-carbonitrile 18g (0.15 g, 88%, yellow solid); ¹H NMR (300 MHz, DMSO-d₆): δ 9.14 (d, J=1.9 Hz, HA), 8.93 (d, J=0.6 Hz, 1H), 8.77 (s, 1H), 8.37 (s, 1H), 7.70 (d, J=1.9 Hz, 1H), 5.77 (q, J=5.9 Hz, 1H), 3.58-3.46 (m, 1H), 3.30-3.23 (m, 1H), 1.69 (d, J=5.9 Hz, 3H), 1.08 (t, J=7.0 Hz, 3H); MS (ES⁺): 653.0 (2M+H);

### Preparation of 4-chloro-6-nitropyrrolo[1,2-b]pyridazine-3-carbonitrile (18f)

### Step 1:

A stirred solution of 2,2,2-trichloro-1-(1H-pyrrol-2-yl)ethanone [20 g, 94.14 mmol, Prepared from pyrrole 18a using the procedure from Organic Syntheses, Coll. Vol. 6, p.618 (1988); Vol. 51, p. 100 (1971)] and Ac₂O (110 mL) was cooled to −40° C. and treated dropwise with 70% nitric acid (8.24 mL, 128.16 mmol) over 2 h. After completion of addition, the mixture was warmed to room temperature over 2 h and then cooled back down to −40° C. Sufficient ice-water was added to precipitate crude 2,2,2-trichloro-1-(4-nitro-1H-pyrrol-2-yl)ethanone. The residue was filtered and washing with ice-water, dried and purified by flash column chromatography on silica gel (hexanes:ethyl acetate 1:0 to 5:2, R_{f}=0.54 with hexanes:ethyl acetate 5:2) to give 2,2,2-trichloro-1-(4-nitro-1H-pyrrol-2-yl)ethanone (12.5 g, 52%) as a solid; ¹H NMR (300 MHz, DMSO-d₆): δ 13.67 (s, 1H), 8.40 (d, J=1.5 Hz, 1H), 7.71 (d, J=1.52, 1H).

To a solution of 2,2,2-trichloro-1-(4-nitro-1H-pyrrol-2-yl)ethanone (12.47 g, 48.43 mmol) in methanol (26 mL) at room temperature was added NaOMe (17 mL, 25% w/w, 74.29 mmol). The mixture was stirred for 2 h, then quenched with aqueous H₂SO₄ (3 M, 26 mL) and cooled to 0° C. Ice-water was added to precipitate methyl 4-nitro-1H-pyrrole-2-carboxylate 18b (8.07 g, 98%) as a solid; ¹H NMR: (300 MHz, DMSO-d₆): δ 13.19 (s, 1H), 8.07 (d, J=1.68, 1H), 7.31 (d, J=1.65, 1H), 3.83 (s, 3H).

### Step 2:

To a solution of methyl 4-nitro-1H-pyrrole-2-carboxylate 18b (1.0 g, 5.88 mmol) in DMF (50 mL) cooled to -10° C. was added LiHMDS (1 M in THF, 7.1 mL) and stirred at −10° C. for 15 min. To the cold reaction mixture was added O-(diphenylphosphoryl)hydroxylamine (1.8 g, 7.72 mmol) and stirred at room temperature for 20 h. The reaction mixture was diluted with ethyl acetate (200 mL) washed with water (2×100 mL), brine (100 mL), dried over MgSO₄ and filtered. The filtrate was concentrated in vacuo and the residue obtained was purified by column chromatography [silica gel 30 g, eluting with chloroform/methanol, 1:0 to 100:1, (R_{f}=0.59 with chloroform/methanol=100:1)] to furnish methyl 1-amino-4-nitro-1H-pyrrole-2-carboxylate 18c (437 mg, 40%) as a white solid; ¹H NMR (300 MHz, DMSO-d₆): δ 8.08 (d, J=2.3, 1H), 7.26 (d, J=2.3, 1H), 6.72 (s, 2H), 3.82 (s, 3H); MS (ES): 219.9 (M+Cl); Analysis: Calcd for C₆H₇N₃O₄:C, 38.92; H, 3.81; N, 22.70; Found: C, 39.13; H, 3.75; N, 22.66.

### Step 3:

To a solution of methyl 1-amino-4-nitro-1H-pyrrole-2-carboxylate 18c (417 mg, 2.25 mmol) in EtOH (12 mL) was added 3,3-diethoxypropanenitrile 18d (2.9 mL, 95%, 18.36 mmol), 1N HCl (aq. 0.6 mL) and heated at reflux for 15 h. The reaction mixture was cooled to room temperature, treated with DBU (3.8 mL, 24.90 mmol), and stirred at 80° C. for 1 h. The reaction mixture was concentrated in vacuo to remove most of EtOH. The residue obtained was diluted with EtOAc (75 mL), washed with water (50 mL, 30 mL). The combined aqueous solution was acidified with 4N HCl to pH=1 and extracted with chloroform/methanol (3:1, 4×100 mL). The combined extracts were dried over MgSO₄, filtered and the filtrate was concentrated in vacuo. The residue obtained was purified by column chromatography [silica gel 120 g, eluting with chloroform/methanol, 1:0 to 4:1,(R_{f}=0.46 with chloroform/methanol=4:1)] to give 4-hydroxy-6-nitropyrrolo[1,2-b]pyridazine-3-carbonitrile 18e (343 mg) as a brown-purple gum; ¹H NMR (300 MHz, DMSO-d₆): δ 9.58 (s, 1H), 8.21 (d, J=2.2 Hz, 1H), 7.87 (s, 1H), 6.93 (d, J=2.2 Hz, 1H); MS (ES): 203.0 (M−1).

### Step 4:

To a solution of 4-hydroxy-6-nitropyrrolo[1,2-b]pyridazine-3-carbonitrile 18e (320 mg) in acetonitrile (8 mL) was added benzyltriethylammonium chloride (mg, 98%, 3.15 mmol) and N,N-diethylaniline (0.32 mL, 2.50 mmol). The mixture was heated to 80° C. followed by the addition of POCl₃ (0.88 mL, 9.52 mmol). The reaction mixture was stirred at 80° C. for 1 h and then concentrated to dryness. The residue obtained was dissolved in chloroform (200 mL), washed with 1N NaHCO₃ (100 mL), water (100 mL), brine (50 mL), dried over MgSO₄ and filtered. The filtrate was concentrated in vacuo and the residue obtained was purified by column chromatography [silica gel 30 g, eluting with hexanes/ethyl acetate, 1:0 to 5:1, (R_{f}=0.45 with hexanes/ethyl acetate 5:1)] to afford 4-chloro-6-nitropyrrolo[1,2-b]pyridazine-3-carbonitrile 18f (95 mg, 20% for two steps) as a yellow solid; ¹H NMR (300 MHz, DMSO-d₆): δ 9.26 (d, J=1.9 Hz, 1H), 8.84 (s, 1H), 7.75 (d, J=1.9 Hz, 1H).

### EXAMPLE 10

### 4-(1H-Pyrazol-4-yl)pyrrolo[1,2-b]pyridazine-3-carbonitrile (16a)

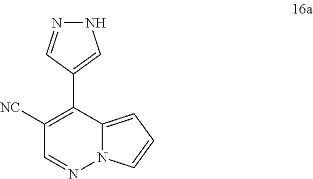 [SMILES: N#Cc1cnn2cccc2c1-c1cn[nH]c1]

To a solution of 4-(1-(1-ethoxyethyl)-1H-pyrazol-4-yl)pyrrolo[1,2-b]pyridazine-3-carbonitrile 14a (188 mg, 0.668 mmol) in THF (6 mL) was added hydrogen chloride (7.00 mL, 7.00 mmol) and stirred at room temperature for 7 h. The reaction mixture was neutralized with 6N aqueous NaOH and concentrated in vacuum to dryness. The residue was triturated with 10 mL of water, and the solid obtained was collected by filtration, dried under vacuum to give 4-(1-(2-cyano-1-cyclopentylethyl)-1H-pyrazol-4-yl)pyrrolo[1,2-b]pyridazine-3-carbonitrile 16a (129 mg, 92%) as a yellow solid; ¹H NMR (300 MHz, DMSO-d₆) δ 13.70 (s, 1H), 8.46 (s, 2H). 8.44 (bs, 1H), 8.17 (dd, J=2.6, 1.4 Hz, 1H), 7.15 ((dd, J=4.5, 1.4 Hz, 1H), 7.10 (dd, J=4.5, 2.6 Hz, 1H); MS (ES−): 208.0 (M−1).

### EXAMPLE 11

### 4-(1-(2-Cyano-1-cyclopentylethyl)-1H-pyrazol-4-yl)pyrrolo[1,2-b]pyridazine-3-carbonitrile (16b)

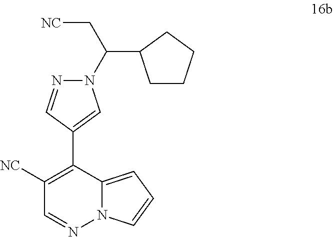 [SMILES: N#CCC(C1CCCC1)n1cc(-c2c(C#N)cnn3cccc23)cn1]

To a solution of 4-(1H-pyrazol-4-yl)pyrrolo[1,2-b]pyridazine-3-carbonitrile 16a (60 mg, 0.287 mmol) in DMF (1.5 mL) was added 3-cyclopentylacrylonitrile (109 mg, 0.717 mmol), 2,3,4,6,7,8,9,10-octahydropyrimido[1,2-a]azepine (0.270 mL, 1.772 mmol) and heated with stirring at 50° C. for 3 h. The reaction mixture was cooled to RT and concentrated in vacuum to dryness. The residue obtained was purified by combiflash column chromatography [silica gel, 4 g eluting with hexanes/ethyl acetate (1:0 to 2:1)] to give 4-(1-(2-cyano-1-cyclopentylethyl)-1H-pyrazol-4-yl)pyrrolo[1,2-b]pyridazine-3-carbonitrile 16b (56 mg, 59%, R_{f}=0.42 with hexanes/ethyl acetate=2:1) as a yellow solid; ¹H NMR (300 MHz, DMSO-d6). δ 8.80 (d, J=0.6 Hz, 1H), 8.48 (s, 1H), 8.31 (s, 1H), 8.19 (t, J=2.1 Hz, 1H), 7.13 (d, J=2.0 Hz, 2H), 4.64 (td, J=9.0, 4.8 Hz, 1H), 3.29-3.21 (m, 2H), 2.48-2.37 (m, 1H), 1.91-1.11 (m, 8H).

### EXAMPLE 12

### Methyl (4-(1-(1-ethoxyethyl)-1H-pyrazol-4-yl)pyrrolo[2,1-f][1,2,4]triazin-2-yl)carbamate (26e)

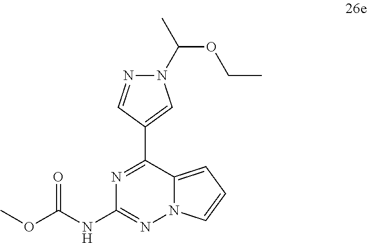 [SMILES: CCOC(C)n1cc(-c2nc(NC(=O)OC)nn3cccc23)cn1]

To a solution of methyl 4-chloropyrrolo[1,2-f][1,2,4]triazin-2-ylcarbamate 26d (300 mg, 1.324 mmol), 1-(1-ethoxyethyl)-4-(4,4,5,5-tetramethyl-1,3,2-dioxaborolan-2-yl)-1H-pyrazole 13a (423 mg, 1.589 mmol), potassium carbonate (732 mg, 5.30 mmol) in 1,4-dioxane/water (12 mL/6 mL) was treated with tetrakis(triphenylphosphine)palladium (O) (155 mg, 0.132 mmol) under nitrogen and heated at 85° C. for 4 h. The reaction mixture was cooled to room temperature, diluted with ethyl acetate (200 mL), washed with water (100 mL), brine (75 mL), dried over MgSO₄, filtered and concentrated in vacuum. The residue obtained was purified by flash chromatography [silica gel, eluting with hexanes/ethyl acetate (1:0 to 1:1)] to give methyl (4-(1-(1-ethoxyethyl)-1H-pyrazol-4-yl)pyrrolo[2,1-f][1,2,4]triazin-2-yl)carbamate 26e (382 mg, 87%, R_{f}=0.20 with hexanes/ethyl acetate=1:1) as a yellow solid. ¹H NMR (300 MHz, DMSO-d6) δ 10.16 (s, 1H), 8.88 (s, 1H), 8.38 (s, 1H), 7.95 (dd, J=2.5, 1.4 Hz, 1H), 7.40 (dd, J=4.6, 1.4 Hz, 1H), 6.96 (dd, J=4.6, 2.5 Hz, 1H), 5.71 (q, J=5.9 Hz, 1H), 3.68 (s, 3H), 3.56-3.42 (m, 1H), 3.32-3.20 (m, 1H), 1.68 (d, J=5.9 Hz, 3H), 1.07 (t, J=7.0 Hz, 3H), MS (ES⁺): 331.1 (M+1).

### Preparation of methyl 4-chloropyrrolo[1,2-f][1,2,4]triazin-2-ylcarbamate (26d)

### Step 1:

To a solution of methyl 1-amino-1H-pyrrole-2-carboxylate 11b (0.29 g, 2.1 mmol) in methanol/AcOH (5 mL/0.6 mL) was added S-methyl bis(methoxycarbonyl)thiourea 26a (0.47 g, 2.28 mmol) and stirred at room temperature for 16 h. The reaction mixture was diluted with ether (5 mL) and hexane (15 mL). The solid obtained was collected by filtration, washed with hexane and dried under vacuum to furnish methyl 1-(2,3-bis(methoxycarbonyl)guanidino)-1H-pyrrole-2-carboxylate 26b (0.5 g, 81%) as a white solid; mp 160.3° C. ¹H NMR (300 MHz, DMSO) δ 11.17-10.23 (m, 1H), 10.16-9.48 (m, 1H), 7.10-6.86 (m, 1H), 6.79 (s, 1H), 6.11 (s, 1H), 3.70 (s, 3H), 3.66 (s, 3H), 3.49 (s, 3H). MS ES(+) 299.1 (M+1); ES(−) 296.9 (M−1); Analysis: Calcd for C₁₁H₁₄N₄O₆: C, 44.30; H, 4.73; N, 18.79; Found: C, 44.21; H, 4.76; N, 18.72.

### Step 2:

To a solution of methyl 1-(2,3-bis(methoxycarbonyl)guanidino)-1H-pyrrole-2-carboxylate 26b (0.145 g, 0.5 mmol) in methanol (5 mL) was added NaOMe (25% wt, 1.08 mL, 5 mmol) and stirred at room temperature for 16 h. The reaction mixture was concentrated under vacuum and the residue obtained was triturated with water. The solid obtained was collected by filtration and dried under vacuum to furnish methyl (4-oxo-3,4-dihydropyrrolo[2,1-f][1,2,4]triazin-2-yl)carbamate 26c (0.087 g, 84%) as an off-white solid; mp 232.4° C.; ¹H NMR (300 MHz, DMSO) δ 11.00 (s, 2H), 7.50 (dd, J=1.7, 2.6 Hz, 1H), 6.89 (dd, J=1.7, 4.4 Hz, 1H), 6.50 (dd, J=2.6, 4.4 Hz, 1H), 3.72 (s, 3H); Analysis:Calcd for C₈H₈N₄O₃: C, 46.16; H, 3.87; N, 26.91; Found:C, 46.07; H, 3.85; N, 26.88.

### Step 3:

To a solution of methyl (4-oxo-3,4-dihydropyrrolo[2,1-f][1,2,4]triazin-2-yl)carbamate 26c (1.9 μm, 9.12 mmol) in acetonitrile (75mL) was added benzyltriethylammonium chloride (4.15 gm, 18.24 mmol) and N,N-diethylaniline (2.17 gm, 14.6 mmol). The reaction mixture was heated to 80° C., to the heat reaction mixture was added dropwise POCl₃ (11.18 gm, 72.96 mmol) and continued heating for 15 h. The reaction mixture was cooled to room temperature and concentrated in vacuum to dryness. The residue obtained was taken in ethyl acetate (400 mL), washed with aqueous NaHCO₃ (1N, 20 0 mL), water (200 mL0, brine (100 mL), dried, filtered and concentrated in vacuum. The residue obtained was purified by flash column chromatography (silica gel, eluting with ethylacetate/hexanes) to afford methyl (4-chloropyrrolo[2,1-f][1,2,4]triazin-2-yl)carbamate 26d (1.05 μm, 50%) as a light yellow solid; ¹H NMR (300 MHz, DMSO) δ 10.55 (s, 1H), 8.10 (dd, J=2.5, 1.5 Hz, 1H), 7.04 (dd, J=4.7, 1.5 Hz, 1H), 6.98 (dd, J=4.7, 2.5 Hz, 1H), 3.68 (s, 3H); MS (ES+) 227.1 (M+1).

### EXAMPLE 13

### 4-(1-(1-Ethoxyethyl)-1H-pyrazol-4-yl)pyrrolo[2,1-f][1,2,4]triazin-2-amine (26k)

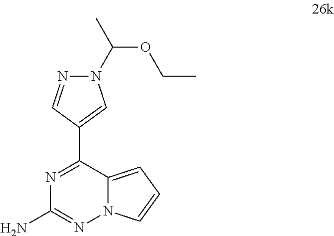 [SMILES: CCOC(C)n1cc(-c2nc(N)nn3cccc23)cn1]

To a solution of methyl 4-(1-(1-ethoxyethyl)-1H-pyrazol-4-yl)pyrrolo[1,2-f][1,2,4]triazin-2-ylcarbamate 26e (140 mg, 0.424 mmol) in MeOH (7 mL)/THF (5 mL) was added 1 N sodium hydroxide (7.00 mL, 7.00 mmol) and heated at reflux for 5 h. The reaction mixture was cooled to RT and concentrated in vacuum to remove THF and methanol. The aqueous residue was diluted with water (15 mL), extracted with ethyl acetate (2×40 mL). The organic layers were combined washed with brine (15 mL), dried over MgSO₄, filtered and concentrated in vacuum to give 4-(1-(1-ethoxyethyl)-1H-pyrazol-4-yl)pyrrolo[2,1-f][1,2,4]triazin-2-amine 26k (98 mg, 85%) as a yellow solid. ¹H NMR (300 MHz, DMSO-d6) δ 8.78 (s, 1H), 8.29 (s, 1H), 7.59 (dd, J=2.5, 1.4 Hz, 1H), 7.13 (dd, J=4.6, 1.4 Hz, 1H), 6.66 (dd, J=4.6, 2.5 Hz, 1H), 6.10 (s, 2H), 5.69 (q, J=5.9 Hz, 1H), 3.55-3.42 (m, 1H), 3.32-3.18 (m, 1H), 1.68 (d, J=5.9 Hz, 3H), 1.06 (t, J=7.0 Hz, 3H).

### EXAMPLE 14

### 4-(1H-Pyrazol-4-yl)pyrrolo[2,1-[2,1-f][1,2,4]triazin-2-amine (26g)

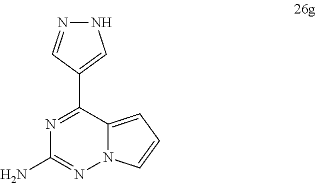 [SMILES: Nc1nc(-c2cn[nH]c2)c2cccn2n1]

To a solution of 4-(1-(1-ethoxyethyl)-1H-pyrazol-4-yl)pyrrolo[2,1-f][1,2,4]triazin-2-amine 26k (77 mg, 0.283 mmol) in THF (5 mL) was added hydrogen chloride (2.90 mL, 2.90 mmol) and stirred at RT for 6 h. The reaction mixture was neutralized with 6 N aqueous NaOH and concentrated in vacuum to dryness. The residue was triturated with water (4 mL), collected by filtration, washed with water, and dried under vacuum to furnish a yellow solid. The yellow solid obtained was purified by flash column chromatography [silica gel 4 g, eluting with chloroform/methanol (1:0 to 9:1)] to afford 4-(1H-pyrazol-4-yl)pyrrolo[2,1-f][1,2,4]triazin-2-amine 26g (9 mg, 16%, R_{f}=0.48 with chloroform/methanol=9:1) as a yellow solid; ¹H NMR (300 MHz, MeOH-d4) δ 8.46 (s, 2H), 7.54 (dd, J=2.3, 1.5 Hz, 1H), 7.09 (dd, J=4.7, 1.3 Hz, 1H), 6.72 (dd, J=4.7, 2.4 Hz, 1H); MS (ES⁺): 201.1 (M+1).

### EXAMPLE 15

The following illustrate representative pharmaceutical dosage forms, containing a compound of formula I (‘Compound X’), for therapeutic or prophylactic use in humans.

| (i) Tablet 1 | mg/tablet |
|---|---|
| Compound X= | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| Compound X= | 20.0 |
| Microcrystalline cellulose | 410.0 |
| Starch | 50.0 |
| Sodium starch glycolate | 15.0 |
| Magnesium stearate | 5.0 |
| | 500.0 |

| ii) Capsule | mg/capsule |
|---|---|
| Compound X= | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/ml) | mg/ml |
|---|---|
| Compound X= (free acid form) | 1.0 |
| Dibasic sodium phosphate | 12.0 |
| Monobasic sodium phosphate | 0.7 |
| Sodium chloride | 4.5 |
| 1.0 N Sodium hydroxide solution | |
| (pH adjustment to 7.0-7.5) | q.s. |
| Water for injection | q.s. ad 1 mL |

| (v) Injection 2 (10 mg/ml) | mg/ml |
|---|---|
| Compound X= (free acid form) | 10.0 |
| Monobasic sodium phosphate | 0.3 |
| Dibasic sodium phosphate | 1.1 |
| Polyethylene glycol 400 | 200.0 |
| 01 N Sodium hydroxide solution | |
| (pH adjustment to 7.0-7.5) | q.s. |
| Water for injection | q.s. ad 1 mL |

| (vi) Aerosol | mg/can |
|---|---|
| Compound X= | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

TABLE I
Activity for Representative Compounds of the
Invention for JAK Family of Enzymes

| Compound | Activity |
|---|---|
| 13e | IC₅₀ < 5 uM |
| 13b | IC₅₀ < 10 uM |
| 13c | IC₅₀ < 10 uM |
| 14c | IC₅₀ > 10 uM |
| 14a | IC₅₀ > 10 uM |
| 14b | IC₅₀ > 10 uM |
| 14d | IC₅₀ < 10 uM |
| 18h | IC₅₀ > 10 uM |
| 16a | IC₅₀ > 10 uM |
| 16b | IC₅₀ < 5 uM |
| 26e | IC₅₀ > 10 uM |
| 26k | IC₅₀ < 10 uM |

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A compound of formula I: 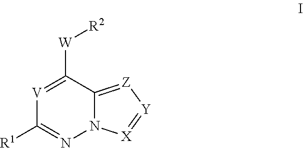
wherein:
W is heteroaryl, heterocycle or aryl, wherein any aryl or heteroaryl of W may be optionally substituted with one or more R_{w} groups and wherein any heterocycle of W may be optionally substituted with one or more groups selected from R_{w} and oxo;
X is N or CRₐ; Y is N or CR_{b}; Z is N or CRₑ; and V is N or CR_{d} provided that no more than two of X, Y, Z or V is N;
R¹ is H, halogen, —(C₁-C₈)alkyl, —(C₂-C₈)alkenyl, —(C₂-C₈)alkynyl, (C₃-C₈)cycloalkyl, aryl, heteroaryl, heterocycle, NO₂, CN, —OH, —ORₑ, —NR_{f}R_{g}, N₃, SH, —SRₑ, —C(O)Rₕ, —C(O)ORₕ, —C(O)NR_{f}R_{g}, —C(═NRₕ)NR_{f}R_{g}, —NRₕCORₑ, —NRₕC(O)ORₑ, —NRₕC(O)OH, —NRₕS(O)₂Rₑ, —NRₕCONR_{f}R_{g}, —OC(O)NR_{f}R_{g}, —S(O)Rₑ, —S(O)NR_{f}R_{g}, —S(O)₂Rₑ, —S(O)₂OH, or —S(O)₂NR_{f}R_{g}, wherein any aryl or heteroaryl of R¹ may be optionally substituted with one or more Rᵢ groups and wherein any —(C₁-C₈)alkyl, (C₃-C₈)cycloalkyl, —(C₂-C₈)alkenyl, —(C₂-C₈)alkynyl or heterocycle of R¹ may be optionally substituted with one or more groups selected from oxo and=NORₕ;
R² is selected from halogen, aryl, heteroaryl, heterocycle, —(C₁-C₈)alkyl, —(C₂-C₈)alkenyl, —(C₂-C₈)alkynyl, (C₃-C₈)cycloalkyl, OH, CN, —OR_{z}, —Oaryl, —Oheterocycle, —Oheteroaryl, —OC(O)R_{z}, —OC(O)NR_{z1}R_{z2}, SH, —SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, —CHO, —C(O)R_{z}, —C(O)OH, —C(O)OR_{z}, —C(O)NR_{z1}R_{z2}, —C(O)heteroaryl and —C(O)C(O)R_{z}, wherein any —(C₁-C₈)alkyl, —(C₂-C₈)alkenyl, —(C₂-C₈)alkynyl, aryl, —Oaryl, Oheteroaryl, —Saryl, —Sheteroaryl, —S(O)aryl, —S(O)heteroaryl, —S(O)₂aryl, —S(O)₂heteroaryl, —NHCOaryl, —NHCOheteroaryl, —NHS(O)₂aryl, —C(O)heteroaryl or heteroaryl of R² may be optionally substituted with one or more R_{y} groups and wherein any heterocycle, —Oheterocycle or (C₃-C₈)cycloalkyl of R² may be optionally substituted with one or more groups selected from oxo, ═CHCN and R_{y}; or R² is H;
Rₐ is H, OH, NO₂, CO₂H, CO₂Rₙ₁, —C(O)NRₙRₒ, —C(O)NHNRₙRₒ, —C(O)NHNHCO₂Rₙ₁, —NHS(O)₂Rₙ₁, —NHCO₂Rₙ₁, —NHCORₙ₂, —NRₙRₒ, halogen or —(C₁-C₆)alkyl wherein —(C₁-C₆)alkyl is optionally substituted with one or more Rₚ groups;
R_{b} is H, OH, NO₂, CO₂H, CO₂Rₙ₁, —C(O)NRₙRₒ, —C(O)NHNRₙRₒ, —C(O)NHNHCO₂Rₙ₁, —NHS(O)₂Rₙ₁, —NHCO₂Rₙ₁, —NHCORₙ₂, —NRₙRₒ, halogen or —(C₁-C₆)alkyl wherein —(C₁-C₆)alkyl is optionally substituted with one or more Rₚ groups;
R_{c} is H, OH, NO₂, CO₂H, CO₂Rₙ₁, —C(O)NRₙRₒ, —C(O)NHNRₙRₒ, —C(O)NHNHCO₂Rₙ₁, —NHS(O)₂Rₙ₁, —NHCO₂Rₙ₁, —NHCORₙ₂, —NRₙRₒ, halogen or —(C₁-C₆)alkyl wherein —(C₁-C₆)alkyl is optionally substituted with one or more Rₚ groups;
R_{d} is H, halogen, —(C₁-C₆)alkyl, —(C₂-C₆)alkenyl, —(C₂-C₆)alkynyl, (C₃-C₆)cycloalkyl, aryl, heteroaryl, heterocycle, NO₂, CN, OH, —OR_{q}, —NRᵣRₛ, N₃, —SH, —C(O)(C₁-C₆)alkyl, —C(O)(C₂-C₆)alkenyl, —C(O)(C₂-C₆)alkynyl, —C(O)(C₃-C₆)cycloalkyl, —C(O)aryl, —C(O)heteroaryl, —C(O)heterocycle, —C(O)ORₜ, —C(O)NRᵣRₛ, —C(═NRₜ)NRᵣRₛ, —NRₜCOR_{q}, —NRₜC(O)OR_{q}, —NRₜS(O)₂R_{q}, —NRₜCONRₜRₛ, —OC(O)NRᵣRₛ, —S(O)R_{q}, —S(O)NRᵣRₛ, —S(O)₂R_{q}, —S(O)₂OH, —S(O)₂NRᵣRₛ or —C(═O)C(═O)NH(C₁-C₆)alkyl, wherein any aryl, —C(O)aryl, —C(O)heteroaryl, or heteroaryl of R_{d} may be optionally substituted with one or more R, groups and wherein any —(C₁-C₆)alkyl, (C₃-C₆)cycloalkyl, —(C₂-C₆)alkenyl, —(C₂-C₆)alkynyl, —C(O)(C₁-C₆)alkyl, —C(O)(C₂-C₆)alkenyl, —C(O)(C₂-C₆)alkynyl, —C(O)(C₃-C₆)cycloalkyl, —C(O)heterocycle or heterocycle of R_{d} may be optionally substituted with one or more groups selected from Rᵢ, oxo and ═NORₜ;
Rₑ is —(C₁-C₆)alkyl, —(C₂-C₆)alkenyl, —(C₂-C₆)alkynyl, (C₃-C₆)cycloalkyl, heterocycle, heteroaryl or aryl;
R_{f} and R_{g} are each independently selected from H, —(C₂-C₆)alkenyl, —(C₂-C₆)alkynyl, (C₃-C₆)cycloalkyl, heterocycle and heteroaryl, wherein any —(C₁-C₆)alkyl of R_{f} or R_{g} may be optionally substituted with one or more groups selected from —C(O)OH and OH; or R_{f} and R_{g} together with the nitrogen to which they are attached form a pyrrolidino, piperidino, piperazino, azetidino, morpholino, or thiomorpholino ring;
Rₕ is H, —(C₁-C₆)alkyl, —(C₂-C₆)alkenyl, —(C₂-C₆)alkynyl, (C₃-C₆)cycloalkyl, heterocycle, heteroaryl or aryl;
each R₁ is independently selected from halogen, aryl, heteroaryl, heterocycle, R_{z}, OH, CN, —OR_{z}, —Oaryl, —OC(O)R_{z}, —OC(O)NR_{z1}R_{z2}, SH, —SR_{z}, —Saryl, —Sheteroaryl, —S(O)_{Rz}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, —CHO, —C(O)R_{z}, —C(O)OH, —C(O)OR_{z}, —C(O)NR_{z1}R_{z2} and —C(O)C(O)R_{z}, wherein any aryl, —Oaryl, Saryl, —S(O)aryl, —S(O)₂aryl, —NHCOaryl or —NHS(O)₂aryl of Rᵢ may be optionally substituted with one or more Rₘ groups;
Rⱼ and Rₖ are each independently selected from H, —(C₁-C₆)alkyl, —(C₂-C₆)alkenyl, —(C₂-C₆)alkynyl, (C₃-C₆)cycloalkyl, aryl, aryl(C₁-C₆)alkyl-, heterocycle and heteroaryl; or Rⱼ and Rₖ together with the nitrogen to which they are attached form a pyrrolidino, piperidino, piperazino, azetidino, morpholino, or thiomorpholino ring;
each Rₘ is independently halogen, aryl, R_{z}, OH, CN, OR_{z}, —Oaryl, —Oheteroaryl, —OC(O)R_{z}, —OC(O)NR_{z1}R_{z2}, SH, SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, CHO, —C(O)R_{z}, —C(O)OH, —C(O)OR_{z}, —C(O)NR_{z1}R_{z2}, —C(O)C(O)R_{z}, heterocycle or heteroaryl;
Rₙ and Rₒ are each independently selected from H, —(C₁-C₆)alkyl, —(C₂-C₆)alkenyl, —(C₂-C₆)alkynyl, (C₃-C₆)cycloalkyl, aryl, aryl(C₁-C₆)alkyl-, heterocycle and heteroaryl, wherein any —(C₁-C₆)alkyl of Rₙ or Rₒ may be optionally substituted with one or more groups selected from —C(O)OH and OH; or Rₙ and Rₒ together with the nitrogen to which they are attached form a pyrrolidino, piperidino, piperazino, azetidino, morpholino, or thiomorpholino ring;
each Rₙ₁ is independently selected from —(C₁-C₆)alkyl, —(C₂-C₆)alkenyl, —(C₂-C₆)alkynyl, (C₃-C₆)cycloalkyl, aryl, aryl(C₁-C₆)alkyl-, heterocycle and heteroaryl;
Rₙ₂ is independently selected from —(C₁-C₆)alkyl, —(C₂-C₆)alkenyl, —(C₂-C₆)alkynyl, (C₃-C₆)cycloalkyl, aryl, aryl(C₁-C₆)alkyl-, heterocycle and heteroaryl, wherein any —(C₁-C₆)alkyl, —(C₂-C₆)alkenyl, —(C₂-C₆)alkynyl, (C₃-C₆)cycloalkyl, aryl, aryl(C₁-C₆)alkyl-, heterocycle or heteroaryl of Rₙ₂ may be optionally substituted with one or more halogens;
each Rₚ is independently selected from halogen, aryl, heteroaryl, heterocycle, R_{z}, OH, CN, —OR_{z}, —Oaryl, —OC(O)R_{z}, —OC(O)NR_{z1}R_{z2}, oxo, SH, SR_{z}, —Saryl, —Sheteroaryl, —S(O)R_{z}, —S(O)aryl, —S(O)heteroaryl, —S(O)₂OH, —S(O)₂R_{z}, —S(O)₂aryl, —S(O)₂heteroaryl, —S(O)₂NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, ═NOR_{z}, —CHO, —C(O)R_{z}, —C(O)OH, —C(O)OR_{z}, —C(O)NR_{z1}R_{z2} and —C(O)C(O)R_{z}, wherein any aryl, Oaryl, —Saryl, —S(O)aryl, —S(O)₂aryl, —NHCOaryl or —NHS(O)₂aryl of Rₚ may be optionally substituted with one or more R_{y} groups;
R_{q} is —(C₁-C₆)alkyl, —(C₂-C₆)alkenyl, —(C₂-C₆)alkynyl, (C₃-C₆)cycloalkyl, aryl, aryl(C₁-C₆)alkyl-, heterocycle and heteroaryl;
Rᵣ and Rₛ are each independently selected from H, —(C₁-C₆)alkyl, —(C₂-C₆)alkenyl, —(C₂-C₆)alkynyl, (C₃-C₆)cycloalkyl, aryl, aryl(C₁-C₆)alkyl-, heterocycle and heteroaryl; or Rᵣ and Rₛ together with the nitrogen to which they are attached form a pyrrolidino, piperidino, piperazino, azetidino, morpholino, or thiomorpholino ring;
Rₜ is H, —(C₁-C₆)alkyl, —(C₂-C₆)alkenyl, —(C₂-C₆)alkynyl, (C₃-C₆)cycloalkyl, aryl, aryl(C₁-C₆)alkyl-, heterocycle and heteroaryl;
each R_{w} is independently (C₁-C₆)alkyl, —O(C₁-C₆)alkyl, —C(O)NRⱼRₖ, halogen, CF₃, CN or NHC(O)Rₕ;
each R_{y} is independently halogen, R_{z}, OH, CN, OR_{z}, —Oaryl, —Oheteroaryl, —OC(O)R_{z}, —OC(O)NR_{z1}R_{z2}, SH, SR_{z}, —Saryl, —Sheteroaryl, —S(O)R_{z}, —S(O)aryl, —S(O)heteroaryl, —S(O)₂OH, —S(O)₂OR_{z}, —S(O)₂R_{z}, —OS(O)₂R_{z}, —S(O)₂Oaryl, —S(O)₂aryl, —OS(O)₂aryl, —S(O)₂heteroaryl, —OS(O)₂heteroaryl, —S(O)₂NR_{z1}R_{z2}, —S(O) NR_{z1}R_{z2}, —NR_{z1}R_{z2}, —NHCOR_{z}, —NHCOaryl, —NHCOheteroaryl, —NHCO₂R_{z}, —NHCONR_{z1}R_{z2}, —NHS(O)₂R_{z}, —NHS(O)₂aryl, —NHS(O)₂NH₂, NO₂, ═NOH, ═NOR_{z}, —C(NH₂)(═NCN), CHO, —C(O)R_{z}, —C(O)OH, —C(O)Oaryl, —C(O)OR_{z}, —C(O)NR_{z1}R_{z2}, —C(O)aryl, —OC(O)aryl, —C(O)heteroaryl, —OC(O)heteroaryl, —C(O)C(O)R_{z}, ═CR_{z7}R_{z8}, aryl, heterocycle or heteroaryl, wherein any aryl, Oaryl, —Oheteroaryl, —Saryl, —Sheteroaryl, —S(O)aryl, —S(O)heteroaryl, —S(O)₂Oaryl, —S(O)₂aryl, —OS(O)₂aryl, —S(O)₂heteroaryl, —OS(O)₂heteroaryl, —NHCOaryl, —NHCOheteroaryl, —NHS(O)₂aryl, —C(O)Oaryl, —C(O)aryl, —OC(O)aryl, —C(O)heteroaryl, —OC(O)heteroaryl or heteroaryl of R_{y} is optionally substituted with one or more halogen, R_{z}, (C₂-C₆)alkynyl, —OR_{z}, CN, NR_{z1}R_{z2}, —NO₂, —CHO, —Oaryl, —C(O)OR_{z}, —C(O)OH, —NHCOR_{z}, —NHS(O)₂R_{z}, —NHS(O)₂aryl, —NHS(O)₂heteroaryl, —C(O)NR_{z1}R_{z2}, —NHCONR_{z1}R_{z2}, —NHC(O)OR_{z}, —NHCOaryl, —NHCOheteroaryl, —NHC(O)OR_{z}, —(C₂-C₆)alkynyl, —S(O)₂NR_{z1}R_{z2}, —S(O)₂R_{z}, —S(O)₂aryl, —S(O)₂heteroaryl, —S(O)₂(C₃-C₆)cycloalkyl, —S(O)R_{z}, —S(O)aryl, —S(O)heteroaryl, —S(O)(C₃-C₆)cycloalkyl, —SR_{z}, —S(C₁-C₆)alkyl aryl, heteroaryl or heterocycle, wherein aryl, —Oaryl, —NHS(O)₂aryl, —NHS(O)₂heteroaryl, —NHCOaryl, —NHCOheteroaryl, —S(O)₂aryl, —S(O)₂heteroaryl, 13 S(O)aryl, —S(O)heteroaryl or heteroaryl is optionally substituted with halogen, CF₃, CN or (C₁-C₃)alkyl, and wherein any heterocycle of R_{y} is optionally substituted with one or more oxo, R_{z}, —S(O)₂R_{z}, —S(O)₂aryl, —S(O)₂heteroaryl, —C(O)R_{z}, —C(O)aryl, —C(O)heteroaryl or heteroaryl wherein aryl, —S(O)₂aryl, —S(O)₂heteroaryl, —C(O)aryl, —C(O)heteroaryl or heteroaryl is optionally substituted with one or more halogen or (C₁-C₃)alkyl;
each R_{z} is independently —(C₁-C₆)alkyl or (C₃-C₆)cycloalkyl, wherein —(C₁-C₆)alkyl may be optionally substituted with one or more R_{z4} groups, and wherein (C₃-C₆)cycloalkyl may be optionally substituted with one or more groups selected from R_{z4}, —(C₁-C₆)alkyl, —(C₁-C₆)alkylCN and —(C₁-C₆)alkylOH;
R_{z1} and R_{z2} are each independently selected from H, —(C₁-C₆)alkyl, —(C₂-C₆)alkenyl, —(C₂-C₆)alkynyl, (C₃-C₆)cycloalkyl, aryl, heterocycle and heteroaryl, wherein any —(C₁-C₆)alkyl, —(C₂-C₆)alkenyl or —(C₂-C₆)alkynyl of R_{z1} or R_{z2} may be optionally substituted with one or more R_{z3} groups, and wherein aryl or heteroaryl of R_{z1} or R_{z2} may be optionally substituted with one or more —(C₁-C₆)alkyl or R_{z3} groups, and wherein any heterocycle or (C₃-C₆)cycloalkyl of R_{z1} or R_{z2} may be optionally substituted with or more —(C₁-C₆)alkyl, oxo or R_{z3} groups; or R_{z1} and R_{z2} together with the nitrogen to which they are attached form a cyclic amino optionally substituted with one or more —(C₁-C₆)alkyl, oxo or R_{z3} groups;
each R_{z3} is independently selected from halogen, CN, CF₃, NR_{z5}R_{z6}, OH, —O(C₁-C₆)alkyl, —C(O)NR_{z5}R_{z6}, —C(O)(C₁-C₆)alkyl, aryl, heterocycle and heteroaryl, wherein any heterocycle of R_{z3} may be substituted with one or more —(C₁-C₆)alkyl; and
each R_{z4} is independently selected from halogen, CN, OH, —NR_{z5}R_{z6}, —SCN, —O(C₁-C₆)alkyl, —Sheteroaryl, —S(O)aryl, —S(O)₂aryl, —Oaryl, —C(O)NR_{z5}R_{z6}, (C₃-C₆)cycloalkyl, —CH₂NHCOaryl, —CH₂OCH₂aryl, biphenyl, aryl, heterocycle and heteroaryl, wherein any aryl, heteroaryl, —Sheteroaryl, —S(O)aryl, —S(O)₂aryl, —Oaryl, —CH₂NHCOaryl, —CH₂OCH₂aryl, biphenyl or heterocycle of R_{z4} may be optionally substituted with one or more halogen, CN, —(C₁-C₆)alkyl, —NH₂, —NHheteroaryl, —NHS(O)₂(C₁-C₆)alkyl or —O(C₁-C₆)alkyl;
R_{z5} and R_{z6} are each independently selected from H or —(C₁-C₆)alkyl wherein alkyl is optionally substituted with NH₂; and
R_{z7} and R_{z8} together with the atom to which they are attached form a (C₃-C₆)cycloalkyl;
or a salt thereof.

2. The compound of claim 1 wherein X is CRₐ.

3. (canceled)

4. The compound of claim 2 wherein Rₐ is H, NO₂, CO₂H, CO₂CH₂CH₃, —C(O)NH₂, —C(O)NHNH₂, —C(O)NHNHCO₂tBu, —NHS(O)₂CH₃, —NHCOCF₃, —NH₂ or —NHCH₂CO₂H.

5-7. (canceled)

8. The compound of claim 1 wherein Y is CR_{b}.

9. (canceled)

10. The compound of claim 8 wherein R_{b} is H, NO₂, CO₂H, —NHS(O)₂CH₃, —NHCOCF₃, —NH₂ or —NHCH₂CO₂H.

11-13. (canceled)

14. The compound of claim 1 wherein Z is CH.

15. (canceled)

16. (canceled)

17. The compound of claim 1 wherein V is CR_{d}.

18. (canceled)

19. The compound of claim 17 wherein R_{d} is H, CN or —C(O)NRᵣRₛ.

20-22. (canceled)

23. The compound of claim 1 wherein V is N.

24-29. (canceled)

30. The compound of claim 1 wherein Y and Z are CH.

31. (canceled)

32. The compound of claim 1 wherein R¹ is H, —NH₂, —NHC(O)OCH₃, —NHCH₂C(O)OH, —NHCH₂CH₂C(O)OH, —NHCH(CO₂H)CH₂OH, —NHCH(CO₂H)₂, or —NHS(O)₂CH₃.

33-40. (canceled)

41. The compound of claim 1 wherein W is pyrazolyl, wherein pyrazolyl is optionally substituted with one or more R_{w} groups.

42. (canceled)

43. (canceled)

44. The compound of claim 1 wherein W—R² is:

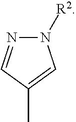

45. The compound of claim 1 wherein R² is absent H.

46-52. (canceled)

53. The compound of claim 1 wherein R² is:

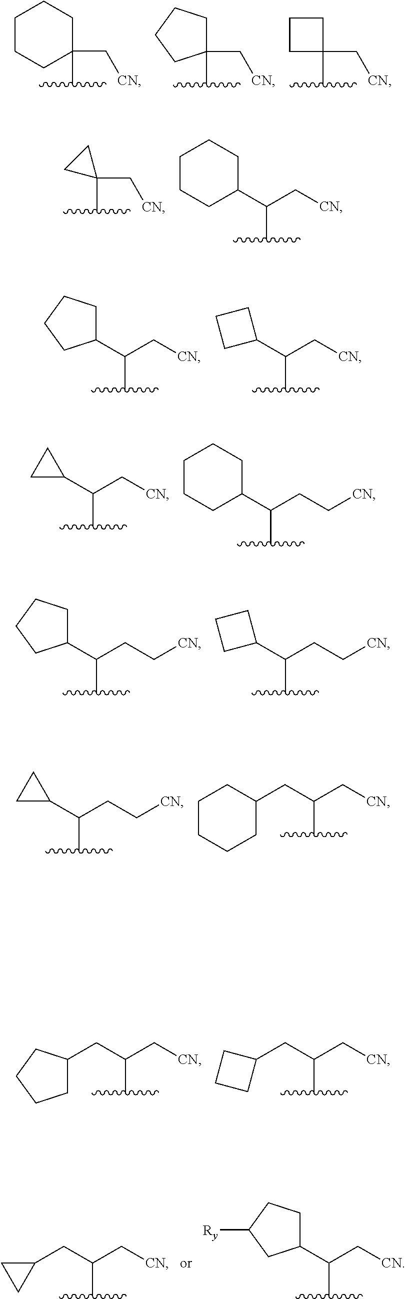

54-61. (canceled)

62. The compound of claim 1 wherein R² is:

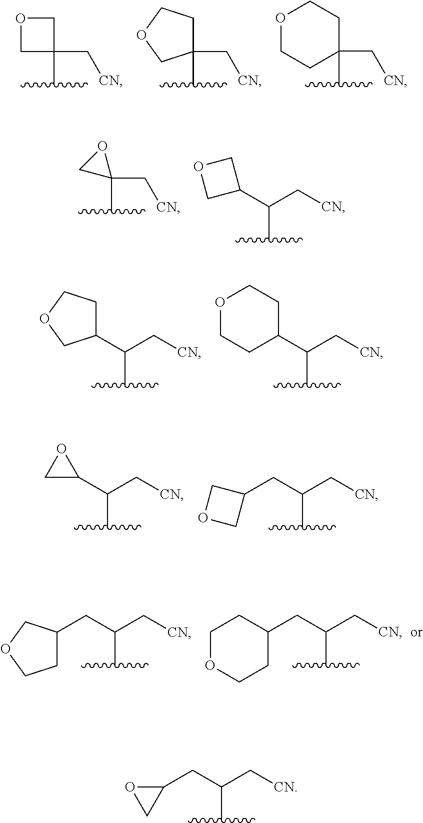

63. The compound of claim 1 wherein R² is: 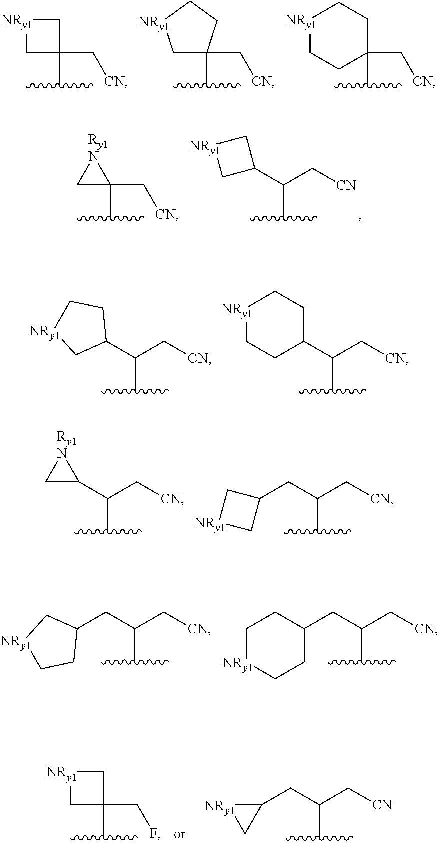
wherein each R_{y1} is independently H, R_{z}, —S(O)₂R_{z}, —S(O)₂aryl, —S(O)₂heteroaryl, —C(O)R_{z}, —C(O)aryl, —C(O)heteroaryl, or heteroaryl wherein any —S(O)₂aryl, —S(O)₂heteroaryl, —C(O)aryl, —C(O)heteroaryl or heteroaryl of R_{y1} is optionally substituted with one or more halogen or (C₁-C₃)alkyl.

64. (canceled)

65. The compound of claim 1 wherein R² is:

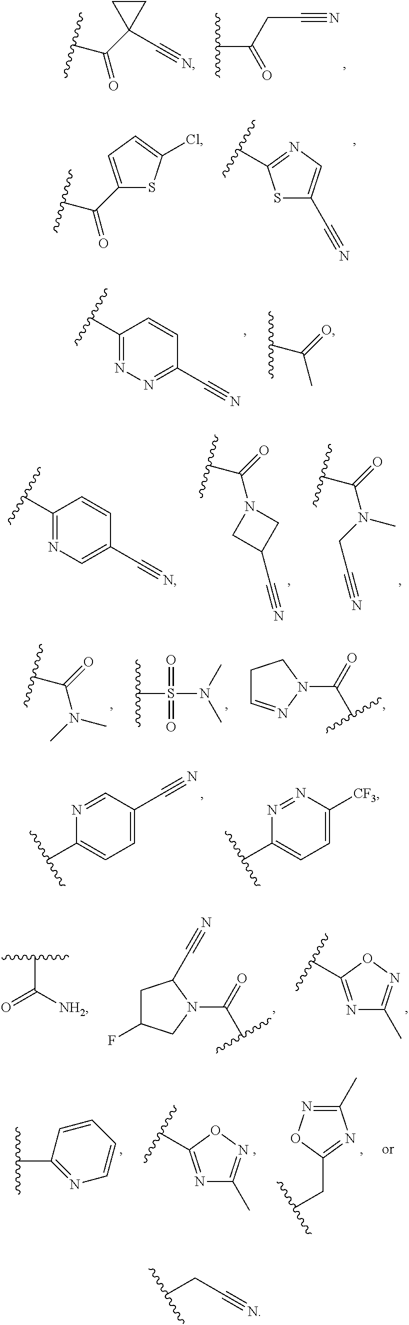

66. The compound of claim 1 wherein R² is:

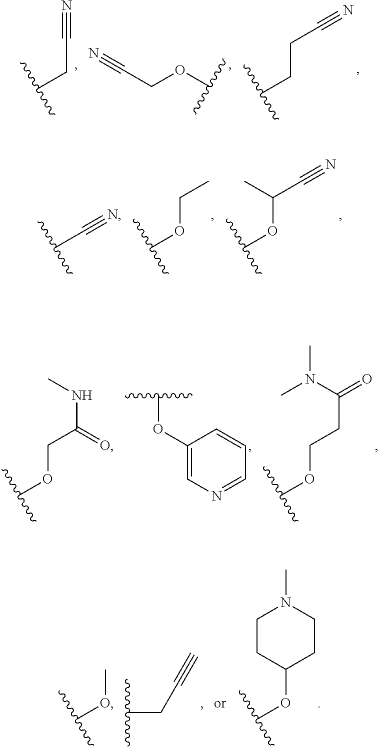

67. The compound of claim 1 wherein R² is:

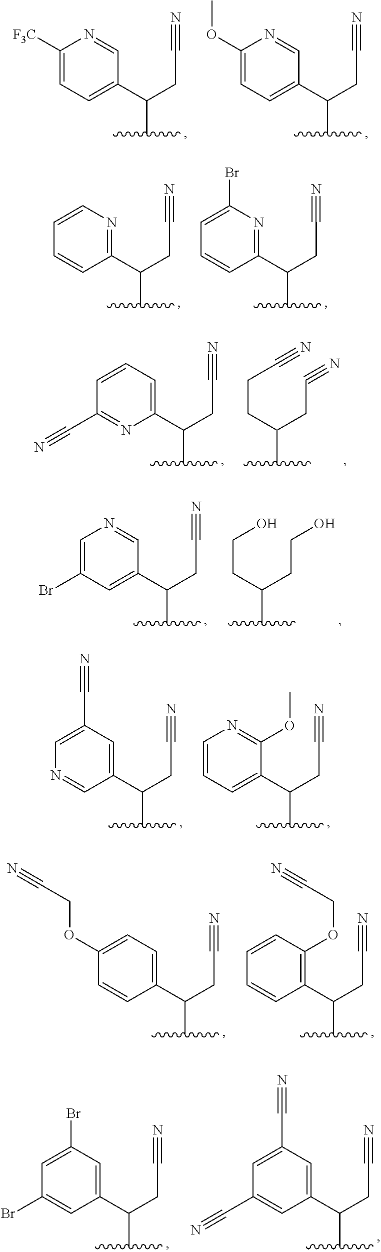

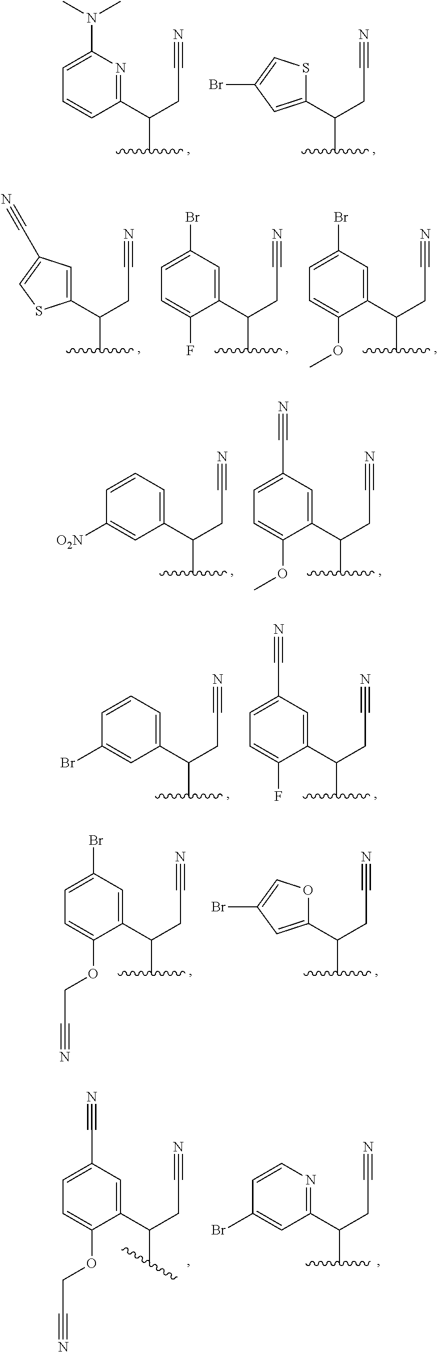

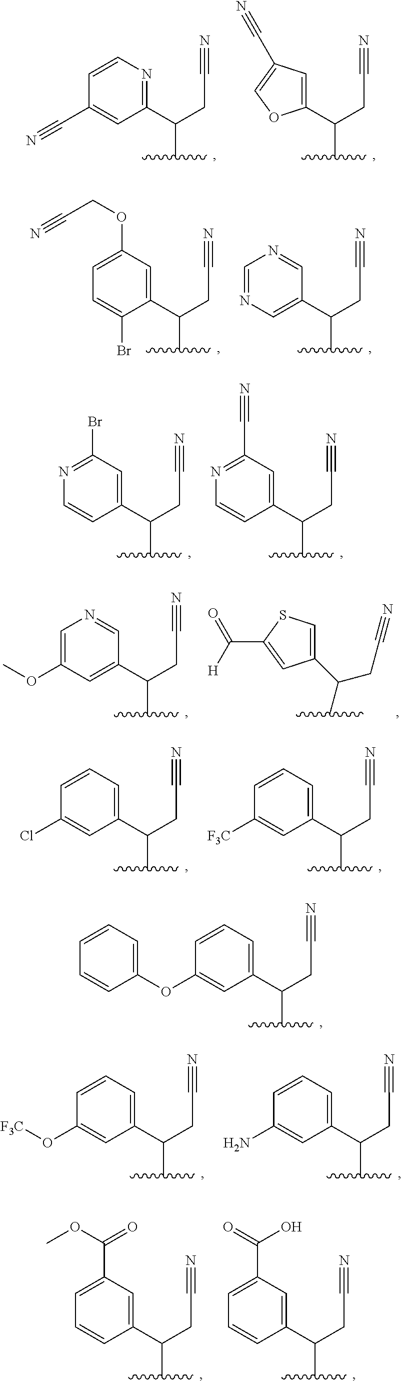

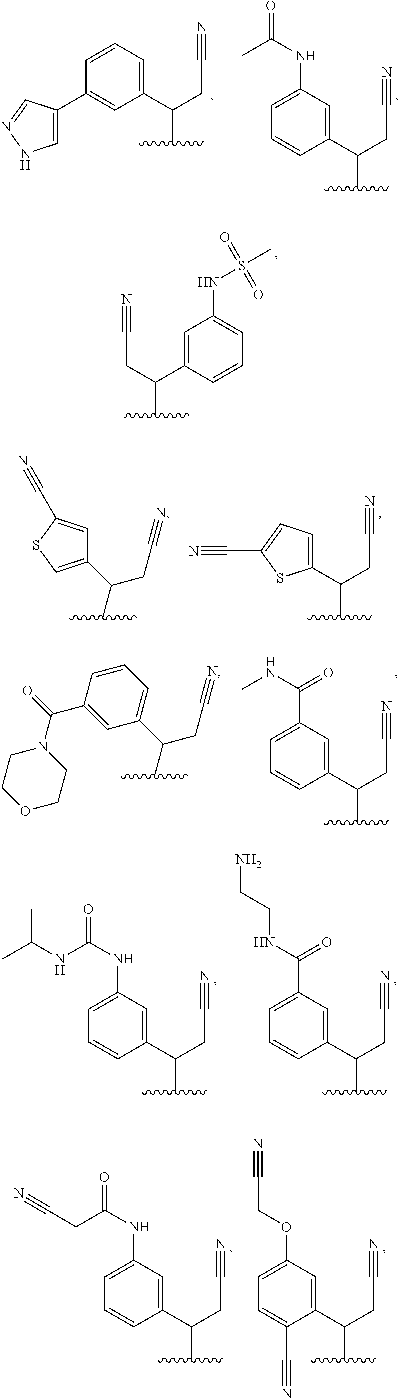

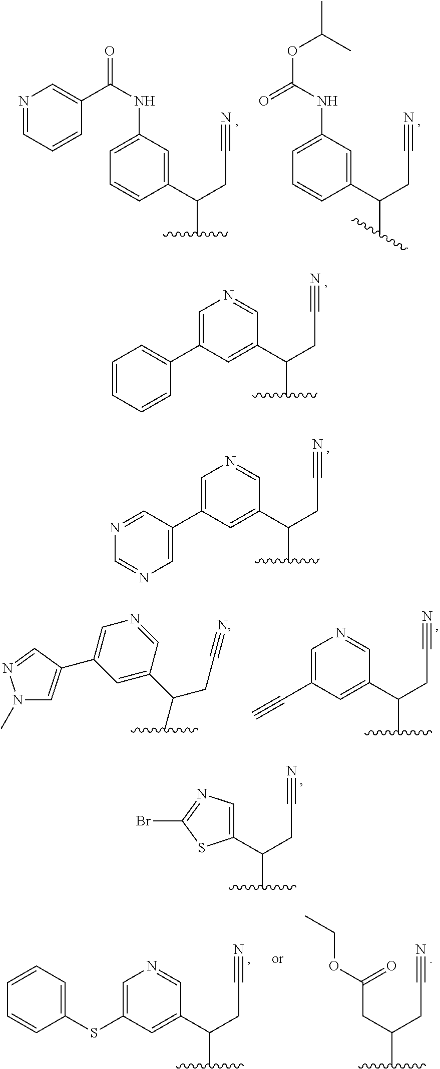

68. The compound of claim 1 wherein W—R² is:

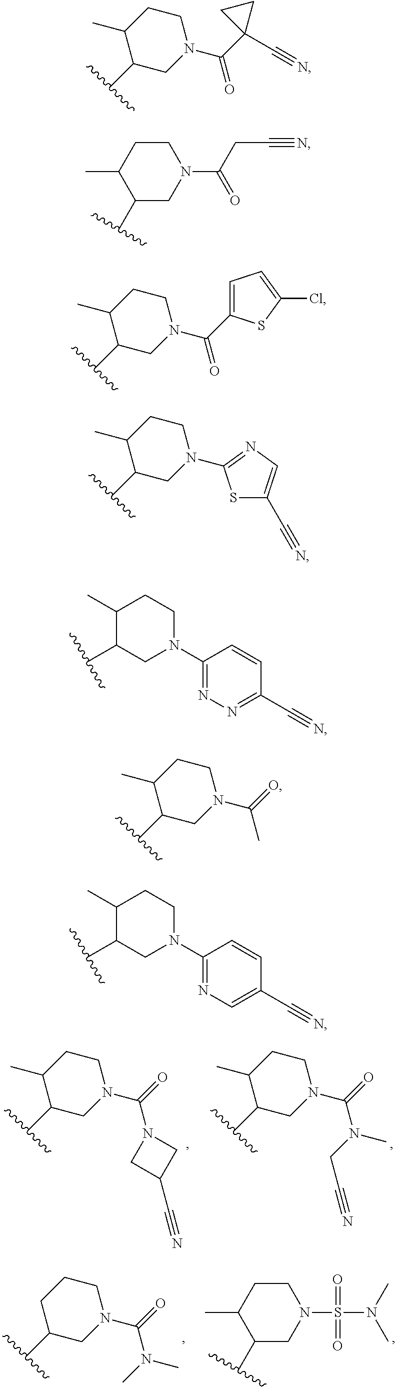

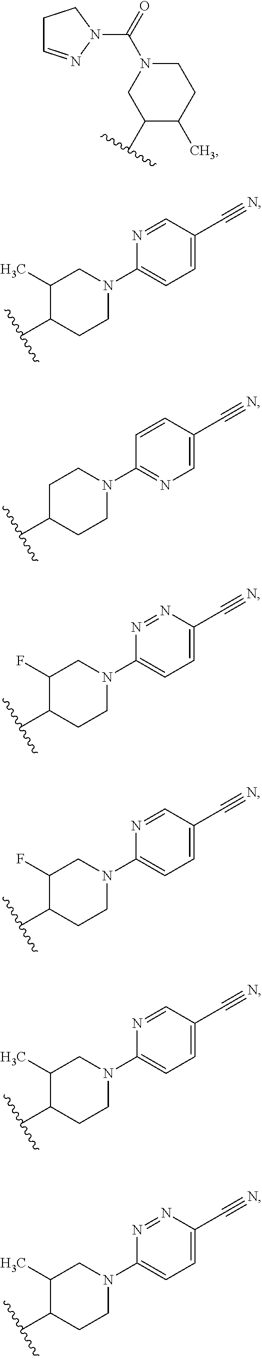

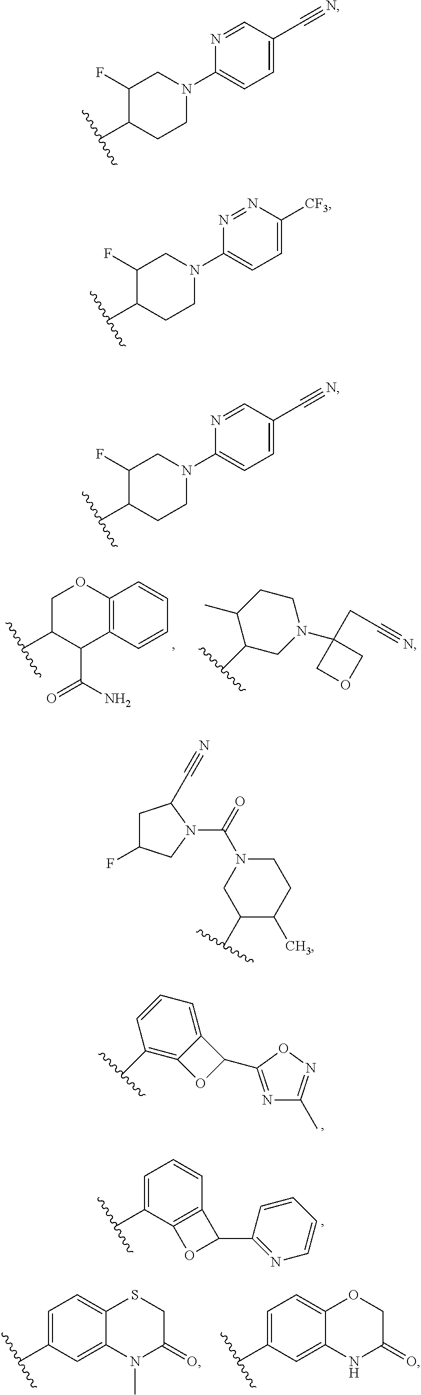

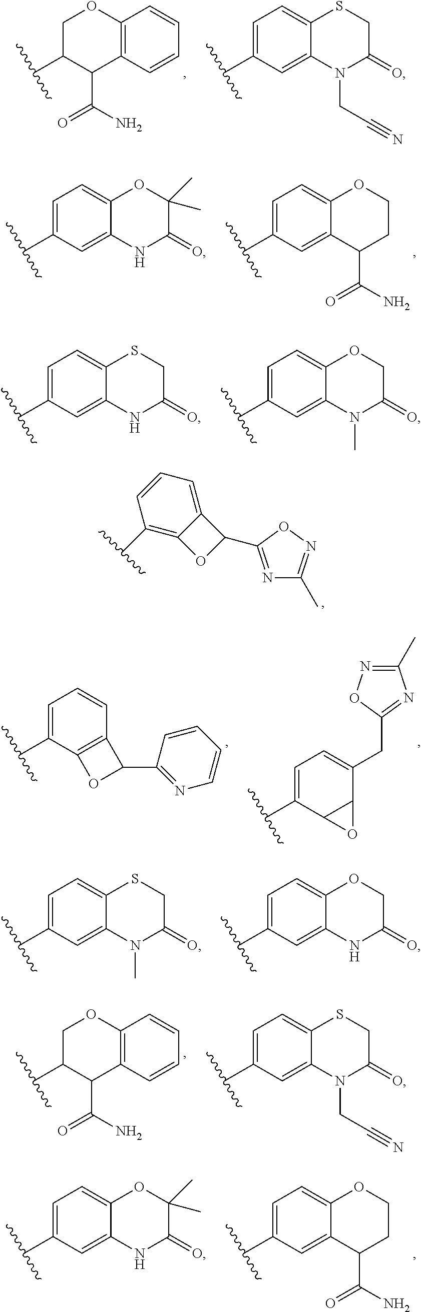

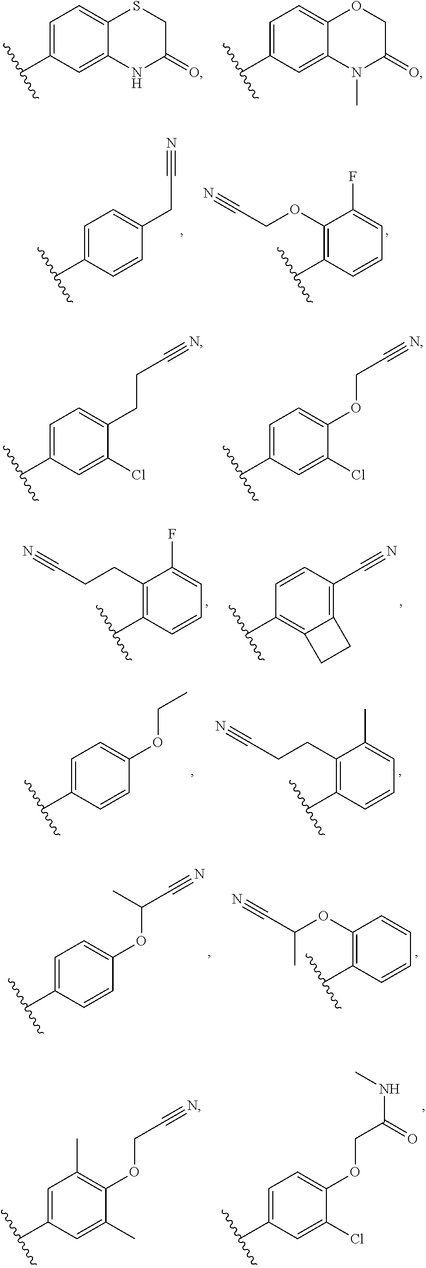

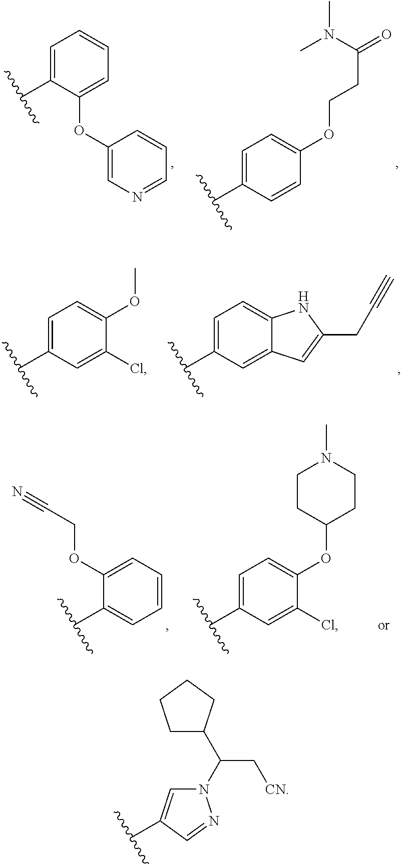

69. The compound of claim 1 wherein W—R² is:

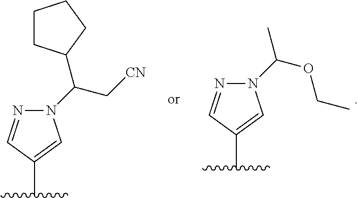 [SMILES: *c1cnn(C(CC#N)C2CCCC2)c1 ; *c1cnn(C(C)OCC)c1]

70. A The compound of claim 1, selected from the group consisting of:
3-cyclopentyl-3-(4-(pyrrolo[1,2-f][1,2,4]triazin-4-yl)-1H-pyrazol-1-yl)propanenitrile;
4-(1-(1-ethoxyethyl)-1H-pyrazol-4-yl)pyrrolo[1,2-f][1,2,4]triazine;
4-(1H-pyrazol-4-yl)pyrrolo[1,2-f][1,2,4]triazine;
4-(1H-pyrazol-4-yl)pyrrolo[1,2-b]pyridazine-3-carboxamide;
4-(1-(1-ethoxyethyl)-1H-pyrazol-4-yl)pyrrolo[1,2-b]pyridazine-3-carbonitrile;
4-(1-(1-ethoxyethyl)-1H-pyrazol-4-yl)pyrrolo[1,2-b]pyridazine-3-carboxamide;
4-(1-(2-cyano-1-cyclopentylethyl)-1H-pyrazol-4-yl)pyrrolo[1,2-b]pyridazine-3-carboxamide;
4-(1-(1-ethoxyethyl)-1H-pyrazol-4-yl)-6-nitropyrrolo[1,2-b]pyridazine-3-carboxamide;
4-(1H-pyrazol-4-yl)pyrrolo[1,2-b]pyridazine-3-carbonitrile;
4-(1-(2-cyano-1-cyclopentylethyl)-1H-pyrazol-4-yl)pyrrolo[1,2-b]pyridazine-3-carbonitrile;
methyl (4-(1-(1-ethoxyethyl)-1H-pyrazol-4-yl)pyrrolo[2,1-f][1,2,4]triazin-2-yl)carbamate;
4-(1-(1-ethoxyethyl)-1H-pyrazol-4-yl)pyrrolo[2,1-f][1,2,4]triazin-2-amine; and
4-(1H-pyrazol-4-yl)pyrrolo[2,1-f][1,2,4]triazin-2-amine,
or a salt thereof.

71. The compound of claim 1, selected from the group consisting of: 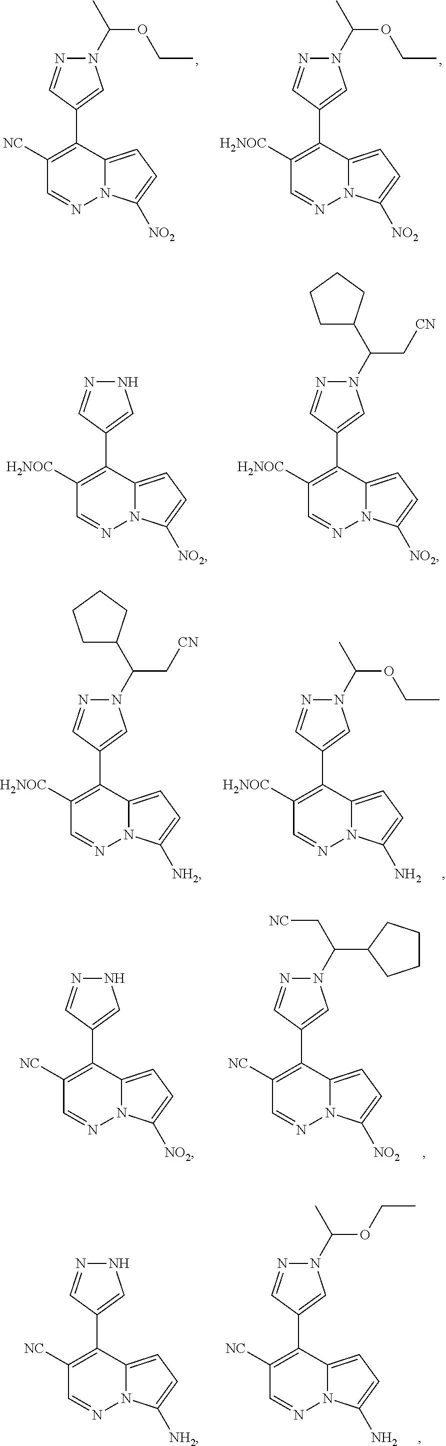 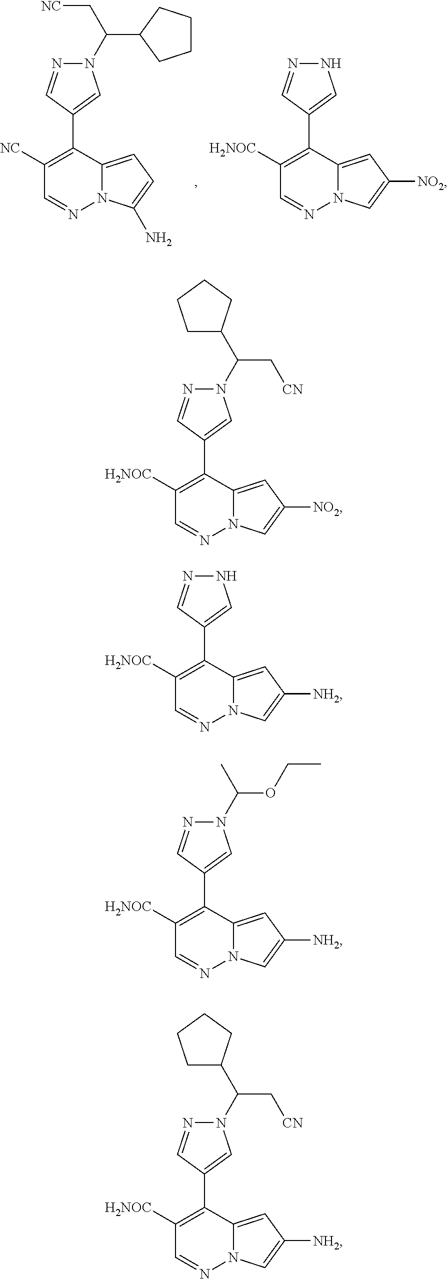 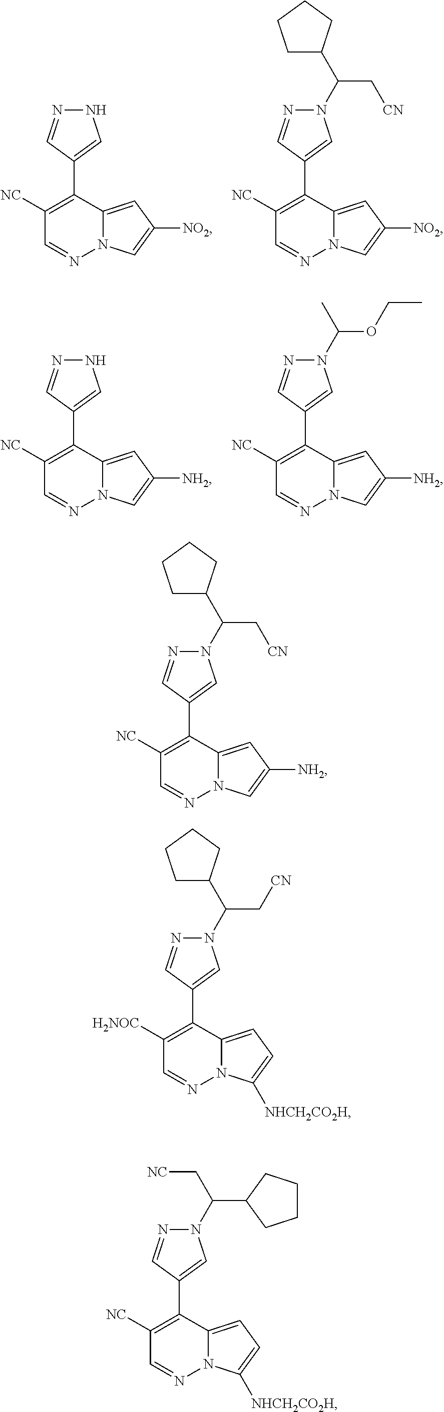 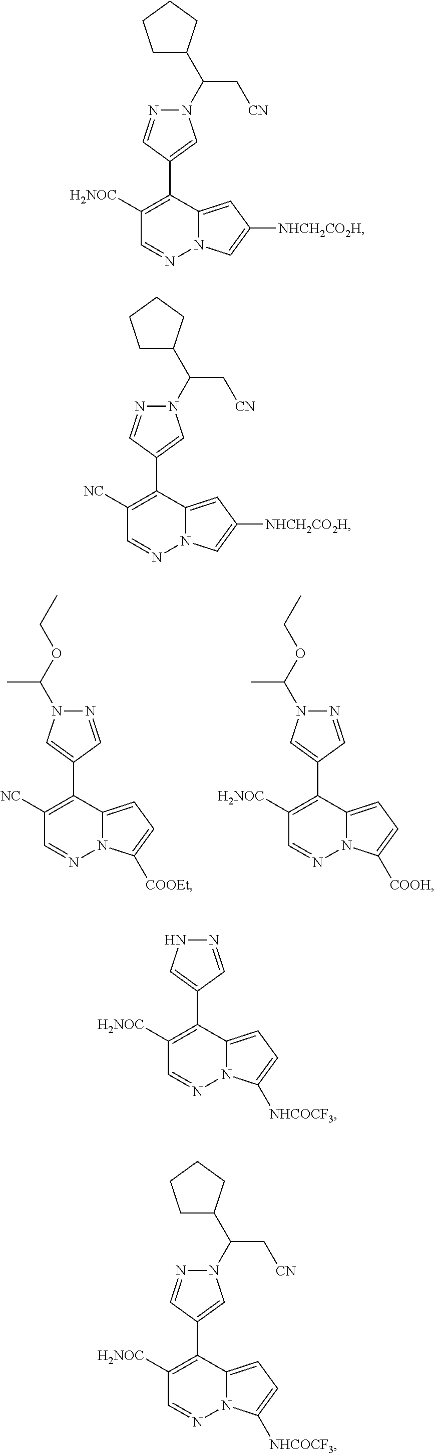 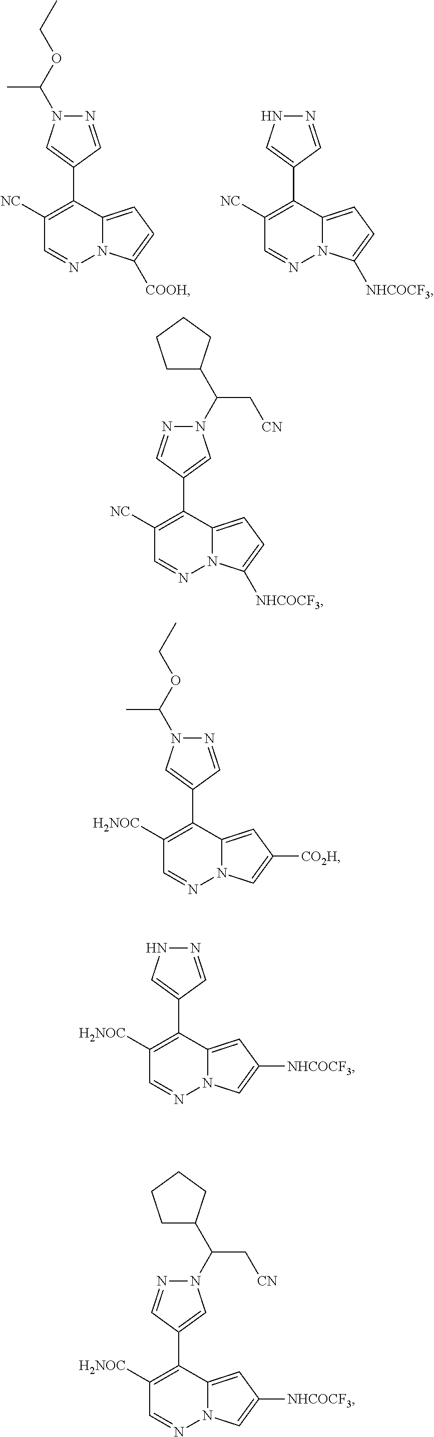 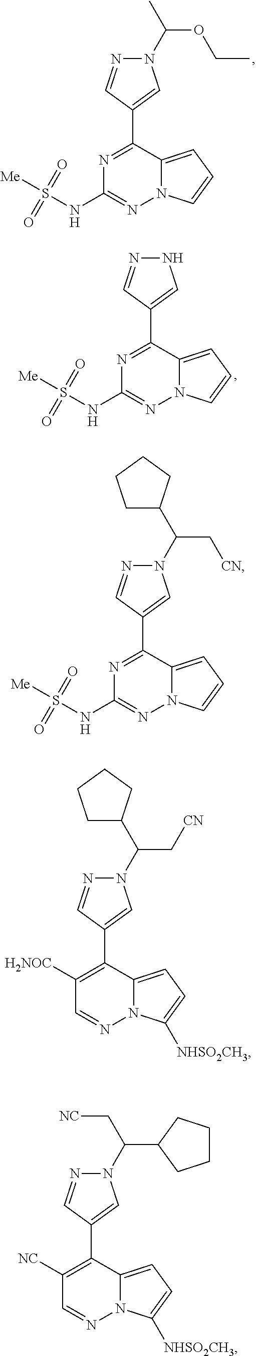 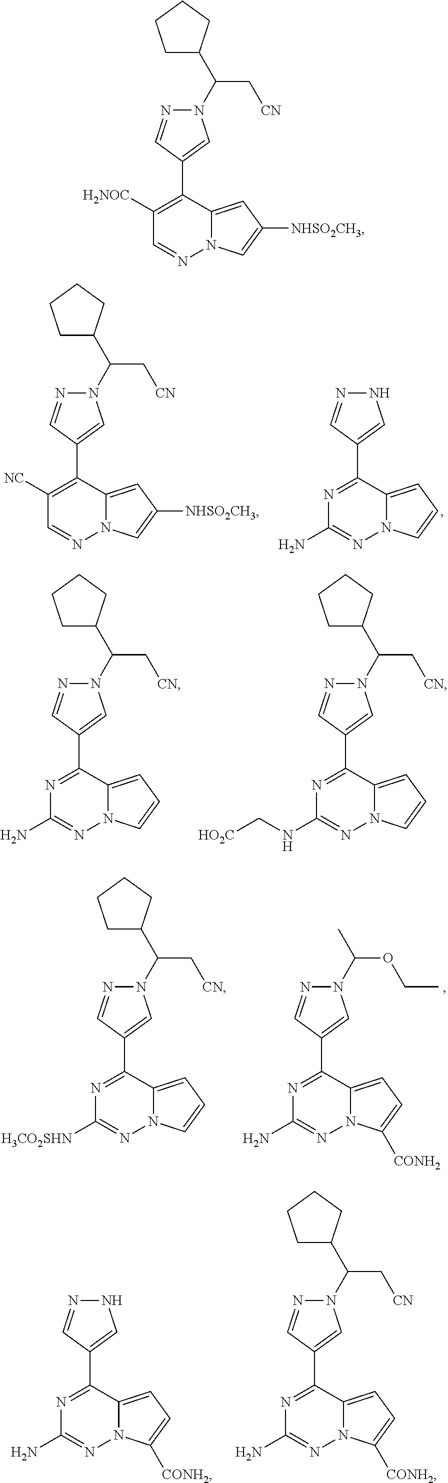 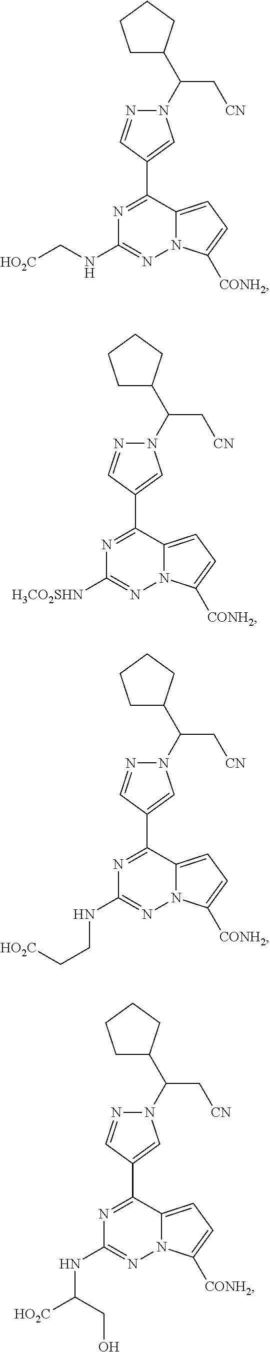 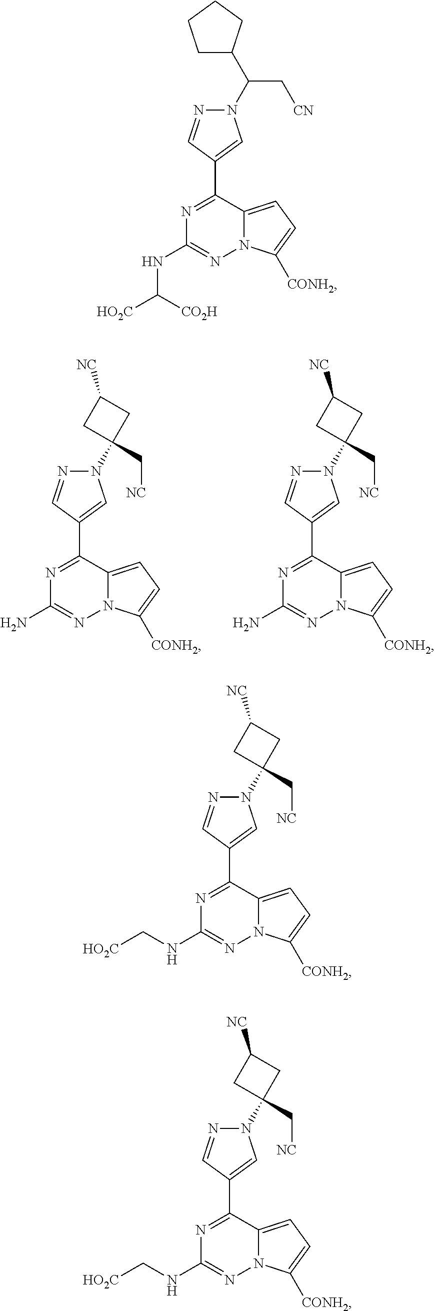 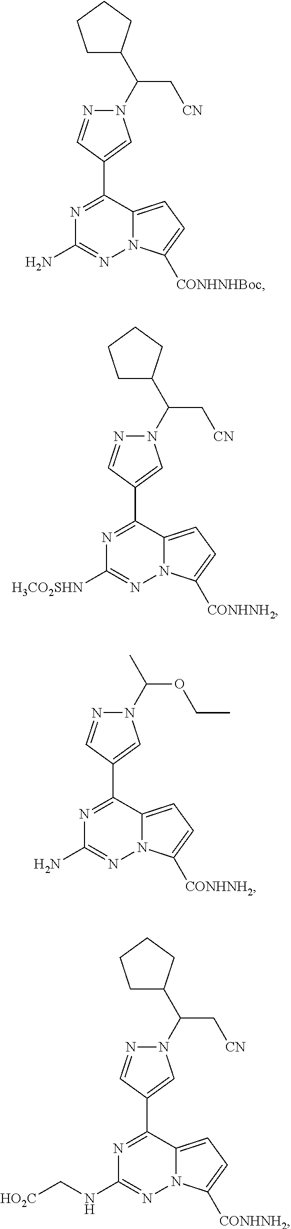 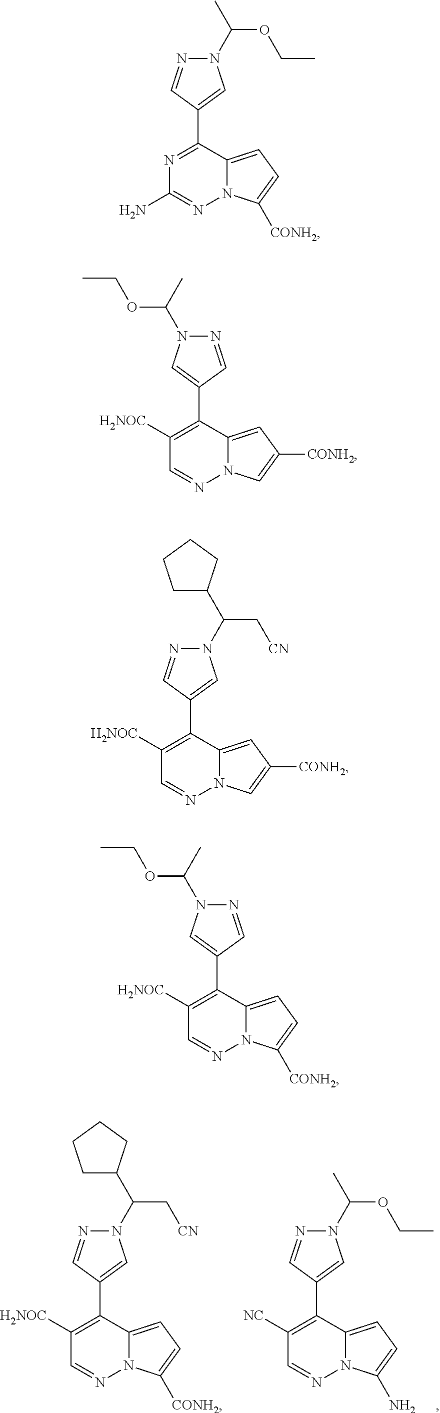 [SMILES: CCOC(C)n1cc(-c2nc(N)nn3c(C(N)=O)ccc23)cn1 ; CCOC(C)n1cc(-c2c(C(N)=O)cnn3cc(C(N)=O)cc23)cn1 ; N#CCC(C1CCCC1)n1cc(-c2c(C(N)=O)cnn3cc(C(N)=O)cc23)cn1 ; CCOC(C)n1cc(-c2c(C(N)=O)cnn3c(C(N)=O)ccc23)cn1 ; N#CCC(C1CCCC1)n1cc(-c2c(C(N)=O)cnn3c(C(N)=O)ccc23)cn1 ; CCOC(C)n1cc(-c2c(C#N)cnn3c(N)ccc23)cn1] 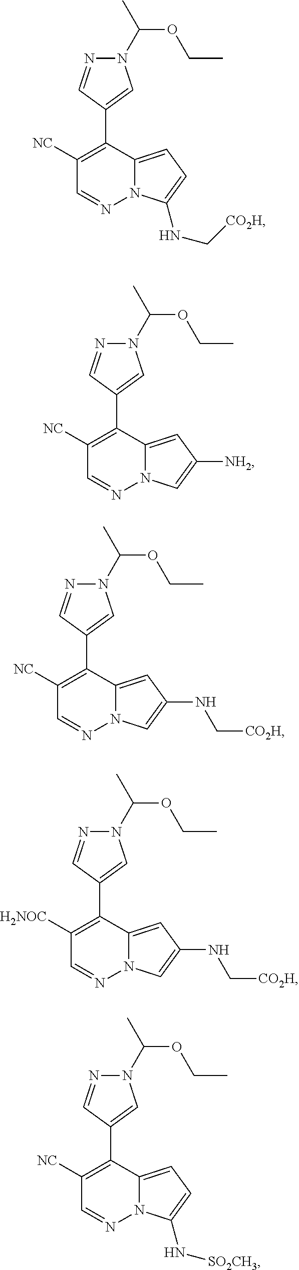 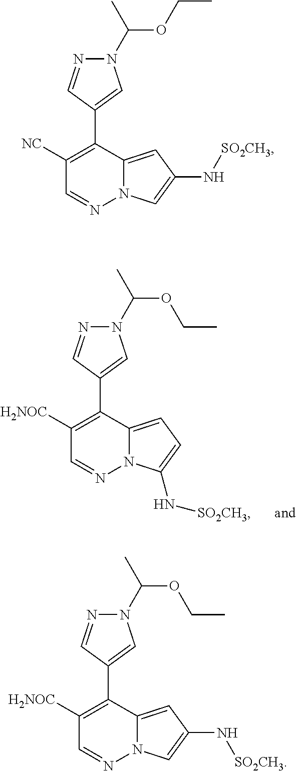 [SMILES: CCOC(C)n1cc(-c2c(C#N)cnn3cc(NS(C)(=O)=O)cc23)cn1 ; CCOC(C)n1cc(-c2c(C(N)=O)cnn3c(NS(C)(=O)=O)ccc23)cn1 ; CCOC(C)n1cc(-c2c(C(N)=O)cnn3cc(NS(C)(=O)=O)cc23)cn1]
or a salt thereof.

72. (canceled)

73. (canceled)

74. A method for treating a disease or condition associated with pathologic JAK activation in a mammal, comprising administering an effective amount of a compound of formula I as described in claim 1, or a pharmaceutically acceptable salt thereof, to the mammal.

75. (canceled)

76. (canceled)

77. The method of claim 74 wherein the disease or condition associated with pathologic JAK activation is cancer.

78. The method of claim 74 wherein the disease or condition associated with pathologic JAK activation is a hematologic or other malignancy.

79. A method for suppressing an immune response in a mammal, comprising administering an effective amount of a compound of formula I as described in claim 1, or a pharmaceutically acceptable salt thereof, to the mammal.

80. (canceled)

81. (canceled)
